(19) United States
(12) Patent Application Publication
(10) Pub. No.: US 2016/0015656 A2
(43) Pub. Date: Jan. 21, 2016

(54) COMPOSITION FOR REGENERATING NORMAL TISSUE FROM FIBROTIC TISSUE

(71) Applicant: Nitto Denko Corporation, Osaka (JP)

(72) Inventors: Yoshiro Niitsu, Sapporo-shi Hokkaido (JP); Akihiro Yoneda, Sapporo-shi Hokkaido (JP); Hirotoshi Ishiwatari, Sapporo-shi, Hokkaido (JP); Joseph E Payne, Oceanside, CA (US); John A Gaudette, Poway, CA (US); Zheng Hou, San Diego, CA (US); Victor Knopov, Oceanside, CA (US); Richard P Witte, San Digeo, CA (US); Mohammad Ahmadian, Carlsbad, CA (US); Loren A Perelman, San Diego, CA (US); Yasunobu Tanaka, Osaka (JP); Violetta Akopian, Oeanside, CA (US)

(21) Appl. No.: 13/789,032

(22) Filed: Mar. 7, 2013

Related U.S. Application Data
(63) Continuation-in-part of application No. 13/813,907, filed on Mar. 19, 2013, now abandoned.
Application No. 13/813,907 is a continuation-in-part of application No. 13/492,424, filed on Jun. 8, 2012, now abandoned.
(60) Provisional application No. 61/494,840, filed on Jun. 8, 2011.

(51) Int. Cl. A61K 31/07; A61K 38/05; A61K 38/48; A61K 45/00

(52) U.S. Cl. CPC A61K 31/07; A61K 45/00; A61K 38/05; A61K 38/4886

(57) ABSTRACT

The present invention relates to a pharmaceutical composition and a method for regenerating normal tissue from fibrotic tissue, the pharmaceutical composition and the method employing a collagen-reducing substance. In accordance with the present invention, normal tissue can be therapeutically regenerated from fibrotic tissue.

### CROSS-REFERENCE TO RELATED APPLICATIONS

This application is a continuation-in-part of U.S. Ser. No. 13/813,907, filed Feb. 1, 2013, which is a national stage filing under 35 U.S.C. §371 of international application PCT/JP2011/067953, filed Aug. 5, 2011. This application is also a continuation-in-part of U.S. Ser. No. 13/492,424, filed Jun. 8, 2012, which claims the benefit of U.S. Provisional Application No. 61/494,840 filed Jun. 8, 2011. The disclosures of all of the above are hereby incorporated by reference in their entireties for all purposes.

### SEQUENCE LISTING

The present application is being filed along with a Sequence Listing in electronic format. The Sequence Listing is provided as a file entitled KUZU1_{—}022P1_SEQ, created Mar. 7, 2013, which is 6 KB in size. The information in the electronic format of the Sequence Listing is incorporated herein by reference in its entirety.

### BACKGROUND OF THE INVENTION

1. Field of the Invention

The present invention relates to a composition and method for regenerating normal tissue from fibrotic tissue. The present invention is further directed to the use of fat-soluble vitamin compounds to target and enhance activity of therapeutic molecules, including siRNA.

2. Description of the Related Art

Fibrosis of tissue is caused by the excessive production and accumulation in tissue of extracellular matrix, which is mainly collagen. When tissue is damaged by a stimulus such as oxidative stress, hypoxia, inflammation, or apoptosis, damaged tissue is repaired by replacement with extracellular matrix, but in the case of the damage being serious or in the case of such stimulation becoming chronic, the accumulation of extracellular matrix becomes excessive, and the tissue cannot perform its function sufficiently. Fibrosis is seen in various types of organs, such as the liver, pancreas, lung, kidney, bone marrow, and heart, and it is thought that collagen-producing cells such as myofibroblasts are related to a disease state.

Conventionally, it is though that fibrosis is an irreversible phenomenon and that once tissue has become fibrotic it does not return to its original state, but recently, there have been some reports suggesting that fibrosis is reversible, and that when the above-mentioned fibrotic stimulus disappears, the extracellular matrix accumulated in the tissue decreases (see Non-Patent Documents 1 to 3).

However, there have been no detailed reports regarding what is specifically happening in the tissue after pathological accumulation of extracellular matrix decreases, and it has been completely unknown until now for regeneration of normal tissue to occur in such fibrotic tissue or for regeneration of normal tissue to be possible.

Furthermore, the fibrosis of tissue not only includes fibroses for which the cause of the disease is clear and can be removed, such as fibrosis derived from viral infection, drinking alcohol, drugs, etc., but also includes fibroses for which the direct cause of the disease is unclear, such as for example cryptogenic cirrhosis, idiopathic pulmonary fibrosis, or idiopathic myelofibrosis, and those for which the direct cause of the disease is known but the origin of the cause of the disease is unclear or is difficult to remove, such as for example primary biliary cirrhosis, nonalcoholic steatohepatitis (NASH)-derived hepatic fibrosis, and primary sclerosing cholangitis. Tissue with the presence of such fibrosis, for which it is difficult to remove the cause of the disease, is in a state in which it is always exposed to a fibrotic stimulus, but it has been completely unknown until now that the pathological accumulation of extracellular matrix in such fibrotic tissue can be reduced, and certainly not known that the tissue can be regenerated.

### CITATION LIST

Non-Patent Document 1: Issa et al., Gastroenterology. 2004; 126(7): 1795-808

Non-Patent Document 2: Iredale, J Clin Invest. 2007; 117(3): 539-48

Non-Patent Document 3: Sato et al., Nat Biotechnol. 2008; 26(4): 431-42

### SUMMARY OF THE INVENTION

### Problems to be Solved by the Invention

It is an object of the present invention to provide a composition and method for therapeutically regenerating normal tissue in tissue in which fibrosis is present.

### Means for Solving the Problems

While carrying out an intensive investigation in order to solve the above-mentioned problems, the present inventors have found that even in fibrotic tissue that continually receives a fibrotic stimulus, collagen accumulated in the tissue can be reduced and, furthermore, normal tissue can be regenerated from the fibrotic tissue by removing the collagen accumulated in the tissue and ensuring there is space in which stem cells can grow and differentiate, and the present invention has thus been accomplished. As described above, although it is known that when a fibrotic stimulus disappears extracellular matrix accumulated in the tissue can decrease, it has been completely unknown until now that in fibrotic tissue that continually receives a fibrotic stimulus collagen accumulated in the tissue can be reduced and that normal tissue can be regenerated from fibrotic tissue by actively removing collagen accumulated in the tissue, and these are surprising findings.

In one aspect, the present invention relates to the following.

(i) A pharmaceutical composition for regenerating normal tissue from fibrotic tissue, the composition containing a collagen-reducing substance.

(ii) The pharmaceutical composition according to (i) above, wherein the collagen-reducing substance is selected from the group consisting of a suppressor of collagen production by collagen-producing cells, a promoter of collagen decomposition, and a suppressor of a collagen decomposition inhibitor.

(iii) The pharmaceutical composition according to (i) or (ii) above, wherein it further contains a targeting agent for collagen-producing cells in fibrotic tissue.

(iv) The pharmaceutical composition according to (iii) above, wherein the targeting agent is a retinoid.

(v) The pharmaceutical composition according to any one of (i) to (iv) above, wherein the fibrotic tissue continually receives a fibrotic stimulus.

(vi) The pharmaceutical composition according to any one of (i) to (v) above, wherein it is for regenerating normal tissue from fibrotic tissue in a space for the growth and differentiation of stem cells, the space being formed by a reduction of collagen accumulated in the fibrotic tissue.

(vii) The pharmaceutical composition according to any one of (ii) to (vi) above, wherein the suppressor of collagen production by collagen-producing cells is selected from the group consisting of a TGFβ inhibitor, HGF or a substance promoting the production thereof, a PPARγ ligand, an angiotensin inhibitor, a PDGF inhibitor, relaxin or a substance promoting the production thereof, a substance that inhibits the production and secretion of an extracellular matrix component, a cell activity supressor, a cell growth supressor, and an apoptosis-inducing substance.

(viii) The pharmaceutical composition according to any one of (ii) to (vi) above, wherein the promoter of collagen decomposition is collagenase or a collagenase production promoter.

(ix) The pharmaceutical composition according to any one of (ii) to (vi) above, wherein the suppressor of a collagen decomposition inhibitor is a TIMP inhibitor.

In one embodiment, the retinoid is provided as a compound containing one or more retinoid moieties, such as a compound consisting of the structure (retinoid)ₘ-linker-(retinoid)ₙ, wherein m and n are independently 0, 1, 2, or 3, except that m and n are not both zero; and wherein the linker comprises a polyethylene glycol (PEG) or PEG-like molecule, or a compound consisting of the structure (lipid)ₘ-linker-(retinoid)ₙ, wherein m and n are independently 0, 1, 2, or 3, except that m and n are not both zero; and wherein the linker comprises a polyethylene glycol (PEG) molecule.

In another aspect, the present invention provides a compound for facilitating drug delivery to a target cell, consisting of the structure (targeting molecule)ₘ-linker-(targeting molecule)ₙ, wherein the targeting molecule is a retinoid having a specific receptor or activation/binding site on the target cell; wherein m and n are independently 0, 1, 2 or 3; and wherein the linker comprises a polyethylene glycol (PEG) or PEG-like molecule. In an embodiment, m and n are not both zero.

In one embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment, the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

In another embodiment, the compound is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

In another embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment, the compound is a composition of formula I wherein q, r, and s are each independently 1, 2, 3, 4, 5, 6, 7, 8, 9, or 10 (see FIG. 31).

In another embodiment, the compound is formula II (see FIG. 32).

In another aspect, the present invention provides a stellate-cell-specific drug carrier comprising a stellate cell specific amount of a retinoid molecule consisting of the structure (retinoid)ₘ-linker-(retinoid)ₙ; wherein m and n are independently 0, 1, 2 or 3; and wherein the linker comprises a polyethylene glycol (PEG) or PEG-like molecule. In an embodiment, m and n are not both zero.

In another embodiment, the present invention provides a composition comprising a liposomal composition. In other embodiments, the liposomal composition comprises a lipid vesicle comprising a bilayer of lipid molecules.

In certain embodiments, the retinoid molecule is at least partially exposed on the exterior of the drug carrier before the drug carrier reaches the stellate cell.

In another embodiment, the retinoid is 0.1 mol % to 20 mol % of the lipid molecules. The retinoid will be present in a concentration of about 0.3 to 30 weight percent, based on the total weight of the composition or formulation, which is equivalent to about 0.1 to about 10 mol %.

The present invention also provides embodiments where the lipid molecules comprise one or more lipids selected from the group consisting of HEDC, DODC, HEDODC, DSPE, DOPE, and DC-6-14. In another embodiment, the lipid molecules further comprise 5104.

In certain embodiments, the drug carrier comprises a nucleic acid.

In other embodiments, the nucleic acid is an siRNA that is capable of knocking down expression of HSP47 mRNA in the stellate cell.

In another aspect, the present invention provides a compound for facilitating drug delivery to a target cell, consisting of the structure (lipid)ₘ-linker-(targeting molecule)ₙ, wherein the targeting molecule is a retinoid or a fat soluble vitamin having a specific receptor or activation/binding site on the target cell; wherein m and n are independently 0, 1, 2 or 3; and wherein the linker comprises a polyethylene glycol (PEG) molecule. In an embodiment, m and n are not both zero.

In one embodiment, the lipid is selected from one or more of the group consisting of DODC, HEDODC, DSPE, DOPE, and DC-6-14.

In another embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment of the present invention, the fat-soluble vitamin is vitamin D, vitamin E, or vitamin K.

In another embodiment, the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

In another embodiment the present invention is selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

Accordingly, the present invention also provides the following:

(1) A compound for facilitating drug delivery to a target cell, consisting of the structure (targeting molecule)ₘ-linker-(targeting molecule)ₙ, wherein the targeting molecule is a retinoid or a fat soluble vitamin having a specific receptor on the target cell; wherein m and n are independently 0, 1, 2, or 3 (except that m and n are not both zero); and wherein the linker comprises a polyethylene glycol (PEG) or PEG-like molecule.

(2) The compound of (1), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(3) The compound of (2), wherein the fat-soluble vitamin is vitamin D, vitamin E, or vitamin K.

(4) The compound of (1), wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

(5) The compound of (1), wherein the compound is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

(6) The compound of (5), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(7) The compound of (6), wherein the compound is a composition of formula I wherein q, r, and s are each independently 1, 2, 3, 4, 5, 6, 7, 8, 9, or 10 (see FIG. 31).

(8) The compound of (6), of formula II (see FIG. 32).

(9) The compound of (1), wherein the PEG is monodisperse.

(10) A stellate-cell-specific drug carrier comprising a stellate cell specific amount of a retinoid molecule consisting of the structure (retinoid)ₘ-linker-(retinoid)ₙ; wherein m and n are independently 0, 1, 2, or 3 (except that m and n are not both zero); and wherein the linker comprises a polyethylene glycol (PEG) or PEG-like molecule.

(11) The drug carrier of (10), further comprising a liposomal composition.

(12) The drug carrier of (11), wherein the liposomal composition comprises a lipid vesicle comprising a bilayer of lipid molecules.

(13) The drug carrier of (11), wherein the retinoid molecule is at least partially exposed on the exterior of the drug carrier before the drug carrier reaches the stellate cell.

(14) The drug carrier of (12), wherein the retinoid is 0.1 mol % to 20 mol % of the lipid molecules.

(15) The drug carrier of (12), wherein the lipid molecules comprise one or more lipids selected from the group consisting of HEDC, DODC, HEDODC, DSPE, DOPE, and DC-6-14.

(16) The drug carrier of (15), wherein the lipid molecules further comprise S104.

(17) The drug carrier of (12), further comprising a nucleic acid.

(18) The drug carrier of (17), wherein the nucleic acid is an siRNA that is capable of knocking down expression of HSP47 mRNA in the stellate cell.

(19) A compound for facilitating drug delivery to a target cell, consisting of the structure (lipid)ₘ-linker-(targeting molecule)ₙ, wherein the targeting molecule is a retinoid or a fat soluble vitamin having a specific receptor on the target cell; wherein m and n are independently 0, 1, 2, or 3 (except that m and n are not both zero); and wherein the linker comprises a polyethylene glycol (PEG) molecule.

(20) The compound of (19), wherein the lipid is selected from one or more of the group consisting of DODC, HEDODC, DSPE, DOPE, and DC-6-14.

(21) The compound of (20), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(22) The compound of (20), wherein the fat-soluble vitamin is vitamin D, vitamin E, or vitamin K.

(23) The compound of (20), wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

(24) The compound of (23), selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

(25) A stellate-cell-specific drug carrier comprising a stellate cell specific amount of a targeting molecule consisting of the molecule (lipid)ₙlinker-(retinoid)ₙ, wherein n=0, 1, 2 or 3 (except that m and n are not both zero); and wherein the linker comprises a polyethylene glycol (PEG) or PEG-like molecule.

(26) The drug carrier of (25), further comprising a liposomal composition.

(27) The drug carrier of (25), wherein the liposomal composition comprises a lipid vesicle comprising a bilayer of lipid molecules.

(28) The drug carrier of (27), wherein the retinoid molecule is at least partially exposed on the exterior of the drug carrier before the drug carrier reaches the stellate cell.

(29) The drug carrier of (27), wherein the retinoid is 0.2 mol % to 20 mol % of the lipid molecules.

(30) The drug carrier of (19), wherein the lipid molecules comprise one or more lipids selected from the group consisting of HEDC, DODC, HEDC, HEDODC, DSPE, DOPE, and DC.

(31) The drug carrier of (30), wherein the lipid molecules further comprise S104.

(32) The drug carrier of (27), further comprising a nucleic acid.

(33) The drug carrier of (32), wherein the nucleic acid is an siRNA that is capable of knocking down expression of HSP47 mRNA in the stellate cell.

### Effects of the Invention

In accordance with the present invention, it has become clear that normal tissue can be regenerated from fibrotic tissue, the regeneration of normal tissue therefrom having been thought not to occur until now. This enables normal tissue to be therapeutically regenerated from fibrotic tissue, and a new regenerative therapy for a fibrotic disease becomes possible.

Furthermore, in accordance with the present invention, it becomes possible to treat fibrotic tissue that is continually exposed to a fibrotic stimulus, and since a medical treatment is realized for all types of fibrotic diseases including a fibrotic disease for which there is no conventional effective therapy and a fibrotic disease for which there is only a treatment involving organ transplantation, an enormous contribution to medical and veterinary treatment can be anticipated.

### BRIEF DESCRIPTION OF THE DRAWINGS

FIG. 1: A photographic diagram showing the overall appearance of livers harvested from test rats and Azan-stained images of representative sections thereof.

FIG. 2: A photographic diagram showing the localization of α-SMA in representative sections of liver harvested from test rats.

FIG. 3: A fluorescence image showing the localization of DAPI and GFP at hepatic stem cell transplantation sites.

FIG. 4: Bright field images and GFP fluorescence images of hepatic stem cell transplantation sites.

FIG. 5A: A photographic diagram comparing DAPI and GFP fluorescence images and an image fluorescently stained by a GFAP antibody in a VA-lip siRNAgp46-treated group (200× magnification).

FIG. 5B: A photographic diagram comparing DAPI and GFP fluorescence images and an image fluorescently stained by a GFAP antibody in a VA-lip siRNAgp46-treated group (400× magnification).

FIG. 6: A 200× magnification photographic diagram comparing DAPI and GFP fluorescence images and an image fluorescently stained by an α-SMA antibody in a VA-lip siRNAgp46-treated group.

FIG. 7: A 200× magnification photographic diagram comparing DAPI and GFP fluorescence images and an image fluorescently stained by an albumin antibody in a VA-lip siRNAgp46-treated group.

FIG. 8: A 200× magnification photographic diagram comparing DAPI and GFP fluorescence images and an image fluorescently stained by a CK19 antibody in a VA-lip siRNAgp46-treated group.

FIG. 9A: A photographic diagram comparing DAPI and GFP fluorescence images and an image fluorescently stained by a ye-CAD antibody in a VA-lip siRNAgp46-treated group (200× magnification).

FIG. 9B: A photographic diagram comparing DAPI and GFP fluorescence images and an image fluorescently stained by a ye-CAD antibody in a VA-lip siRNAgp46-treated group (400× magnification).

FIG. 10: A 200× magnification photographic diagram comparing DAPI and GFP fluorescence images and an image fluorescently stained by an albumin antibody in a site of a VA-lip siRNAgp46-treated group where hepatic stem cells were not transplanted.

FIG. 11: A fluorescence image showing the intracellular distribution of FAM-labeled siRNA in rat pancreatic stellate cells.

FIG. 12: A graph showing the result of a FACS analysis with respect to siRNA incorporated into rat pancreatic stellate cells. Respectively shown in sequence from the top are the results of an untreated group, a Lip siRNAgp46-FAM-treated group, a VA-lip siRNAgp46-FAM-treated group, a VA-lip siRNAgp46-FAM+RBP antibody-treated group, and a Lip siRNAgp46-FAM+RBP antibody-treated group.

FIG. 13: A Western blot image showing the suppression of the expression of gp46 in rat pancreatic stellate cells by siRNAgp46. A shows the difference in suppression effect according to VA-lip siRNAgp46 concentration, and B shows the duration of suppression effect.

FIG. 14: A graph showing the quantitative amounts of collagen produced after 72 hours by untreated cells and cells treated with each of VA-lip siRNAgp46 and VA-lip siRNA random.

FIG. 15: A photographic diagram showing the specific delivery of VA-lip siRNAgp46 to pancreatic stellate cells in DBTC-treated rats. A and B are images of immunostaining by an anti-α-SMA antibody and an anti-FITC antibody of rat pancreatic sections that had been treated three times every other day with VA-lip siRNAgp46-FITC and Lip siRNAgp46-FITC respectively. Staining images a to d on the right-hand side are enlarged images of regions denoted by the corresponding symbols on the staining image on the left-hand side. C shows images of staining by Azan-Mallory staining, anti-α-SMA antibody staining, and anti-FITC antibody staining of rat liver sections that had been treated three times every other day with VA-lip siRNAgp46-FITC. D to F are staining images of staining with an anti-CD68 antibody and an anti-FITC antibody of rat lung, spleen, and retina 24 hours after intravenous administration of VA-lip siRNAgp46-FITC.

FIG. 16: A diagram showing the expression of gp46 protein in the pancreas 0, 1, 2, 3, and 4 days after VA-lip siRNAgp46 administration of rats to which VA-lip siRNAgp46 (siRNA 0.75 mg/kg) was administered on the 14th day after treatment with DBTC. A shows the result of Western blotting of pancreatic cell debris, and B shows the result of a quantitative concentration analysis using β-actin for normalization.

FIG. 17: A diagram showing the effect of VA-lip siRNAgp46 in DBTC-induced pancreatic fibrosis. A shows Azan-Mallory staining images of pancreatic sections of DBTC-treated rat to which one of VA-lip siRNAgp46, Lip siRNAgp46, and PBS was administered 10 times. B is a graph showing quantification by computer image analysis of regions that showed positive in the Azan-Mallory staining images of A. Data were calculated from 6 fields randomly extracted from six rats of each group and are expressed as average values±standard deviation. C is a graph showing the content of hydroxyproline in the pancreas. Data are expressed as average values±standard deviation.

FIG. 18: A diagram showing the effect of VA-lip siRNAgp46 in DBTC-induced pancreatic fibrosis. A shows α-SMA staining images of the pancreas of DBTC-treated rats after treatment with VA-lip siRNAgp46. B is a graph showing quantification by computer image analysis of α-SMA-positive regions in A. Data were calculated from 6 fields randomly extracted from six rats of each group and are expressed as average values±standard deviation.

FIG. 19: A diagram showing the regeneration of normal tissue from fibrotic pancreatic tissue by VA-lip siRNAgp46. A shows hematoxylin-eosin staining images of the pancreas of DBTC-treated rats to which VA-lip siRNAgp46 (right) and Lip siRNAgp46 (left) had been administered 10 times. The bottom diagrams are enlarged diagrams of each region a and b of the top diagrams. B is a graph showing the weight of the pancreas of DBTC-treated rats.

FIG. 20: A graph showing the effect on the differentiation of stem cells in the presence or absence of space around the stem cells. The ordinate shows albumin-positive colony area.

FIG. 21: A graph showing the effect on the differentiation of stem cells in the presence or absence of space around the stem cells. The ordinate shows an index for the growth rate of stem cells.

FIG. 22: VA-conjugate addition to liposomes via decoration enhances siRNA activity

FIG. 23: VA-conjugate addition to liposomes via co-solubilization enhances siRNA activity

FIG. 24: VA-conjugate addition to liposomes via co-solubilization enhances siRNA activity

FIG. 25: VA-conjugate addition to lipoplexes via co-solubilization enhance siRNA activity

FIG. 26: VA-conjugate addition to lipoplexes via co-solubilization vs. decoration.

FIG. 27: in vivo efficacy in mouse, CCl₄ model

FIG. 28: in vivo efficacy of decorated vs. co-solubilized retinoid conjugates

FIG. 29: in vitro efficacy (pHSC), effect of retinoid conjugates in liposome formulations.

FIG. 30: Correlation of retinoid conjugate content (mol %) to in vivo (rat DMNQ) efficacy. Male Sprague-Dawley rats injected intravenously either with formulations containing 0, 0.25, 0.5, 1, and 2% DiVA at a dose of 0.75 mg/kg siRNA, or PBS (vehicle), one hour after the last injection of DMN.

FIG. 31: Structure of formula I.

FIG. 32: Structure of formula II.

FIG. 33: Structure of DOPE-Glu-VA: (Z)-(2R)-3-(((2-(5-(((2E,4E,6E,8E)-3,7-dimethyl-9-(2,6,6-trimethylcyclohex-1-en-1-yl)nona-2,4,6,8-tetraen-1-yl)oxy)-5-oxopentanamido)ethoxy)(hydroxy)phosphoryl)oxy)propane-1,2-diyl dioleate.

FIG. 34: Preparation of DOPE-Glu-VA.

FIG. 35: Structure of DOPE-Glu-NH-VA: (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)phosphoryl)oxy)propane-1,2-diyl dioleate (DOPE-Glu-NH-VA).

FIG. 36: Preparation of DOPE-Glu-NH-VA.

FIG. 37: Structure of DSPE-PEG550-VA: (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate.

FIG. 38: Preparation of intermediate 1 of DSPE-PEG550-VA.

FIG. 39: Preparation of DSPE-PEF550-VA.

FIG. 40: Preparation of DSPE-PEG2000-Glu-VA.

FIG. 41: Structure of DOPE-Gly₃-VA: (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate.

FIG. 42: Preparation of DOPE-Gly₃-VA.

FIG. 43: Structure of VA-PEG-VA: N1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide.

FIG. 44: Preparation of VA-PEG-VA.

FIG. 45: Structure of DSPE-PEG2000-VA.

FIG. 46: Preparation of DSPE-PEG2000-VA.

FIG. 47: Structure of diVA-PEG-diVA: N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide.

FIG. 48: Preparation of Intermediate 1 of diVA-PEG-diVA: Z-DiVA-PEG-DiVA-IN.

FIG. 49: Preparation of Intermediate 2 of diVA-PEG-diVA: DiVA-PEG-DiVA-IN.

FIG. 50: Preparation of DiVA-PEG-DiVA.

FIG. 51: Preparation of DOPE-VA.

FIG. 52: Structure of satDIVA: N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide.

FIG. 53: Preparation of satDIVA.

FIG. 54: Structure of simDIVA: N1,N19-bis((S)-15,22-dioxo-30-(2,6,6-trimethylcyclohex-1-en-1-yl)-16-(9-(2,6,6-trimethylcyclohex-1-en-1-yl)nonanamido)-4,7,10-trioxa-14,21-diazatriacontyl)-4,7,10,13,16-pentaoxanonadecane-1,19-diamide.

FIG. 55: Preparation of simdiVA.

FIG. 56: Structure of DIVA-PEG18: (2E,2′E,2″E,4E,4′E,4″E,6E,6′E,6″E,8E,8′E,8″E)-N,N′,N″-((5R,69R,76E,78E,80E,82E)-77,81-dimethyl-6,68,75-trioxo-83-(2,6,6-trimethylcyclohex-1-en-1-yl)-10,13,16,19,22,25,28,31,34,37,40,43,46,49,52,55,58,61,64-nonadecaoxa-7,67,74-triazatrioctaconta-76,78,80,82-tetraene-1,5,69-triyl)tris(3,7-dimethyl-9-(2,6,6-trimethylcyclohex-1-en-1-yl)nona-2,4,6,8-tetraenamide).

FIG. 57: Structure of TriVA.

FIG. 58: Preparation of Intermediate 3 of TriVA

FIG. 59: Preparation of Intermediate 4 of TriVA

FIG. 60: Preparation of TriVA

FIG. 61: Structure of 4TTNPB

FIG. 62: Structure of 4Myr

FIG. 63: Preparation of 4Myr

FIG. 64: Structure of DiVA-242

FIG. 65: Preparation of DIVA-242

### DETAILED DESCRIPTION OF THE PREFERRED EMBODIMENTS

Within the scope of the invention is a compound for facilitating drug delivery to a target cell, consisting of the structure (targeting molecule)ₘ-linker-(targeting molecule)ₙ, wherein the targeting molecule is a retinoid or a fat soluble vitamin having a specific receptor (or activation/binding site) on the target cell; and wherein m and n are independently 0, 1, 2, or 3 (except that m and n are not both zero); and wherein the linker comprises a polyethylene glycol (PEG) or PEG-like molecule and is designated “Formula A”.

The invention also includes a compound for facilitating drug delivery to a target cell, consisting of the structure (lipid)ₘ-linker-(targeting molecule)ₙ, wherein the targeting molecule is a retinoid or a fat soluble vitamin having a specific receptor on the target cell; wherein m and n are independently 0, 1, 2, or 3 (except that m and n are not both zero); and wherein the linker comprises a polyethylene glycol (PEG) PEG-like molecule and is designated “Formula B”.

It has now been discovered that the compounds of Formula A or Formula B impart properties to the formulations of the invention not previously seen. Formulations of the invention that include compounds of Formula A or Formula B result in superior reduction in gene expression, as compared to formulations that do not include these compounds. Particularly surprising is the ability of formulations of the invention that include compounds of Formula A to reduce the expression of HSP47.

In certain preferred embodiments, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

Preferred embodiments include compounds where the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH. In other embodiments, the PEG is monodisperse.

Another embodiment provides a compound where Formula A is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

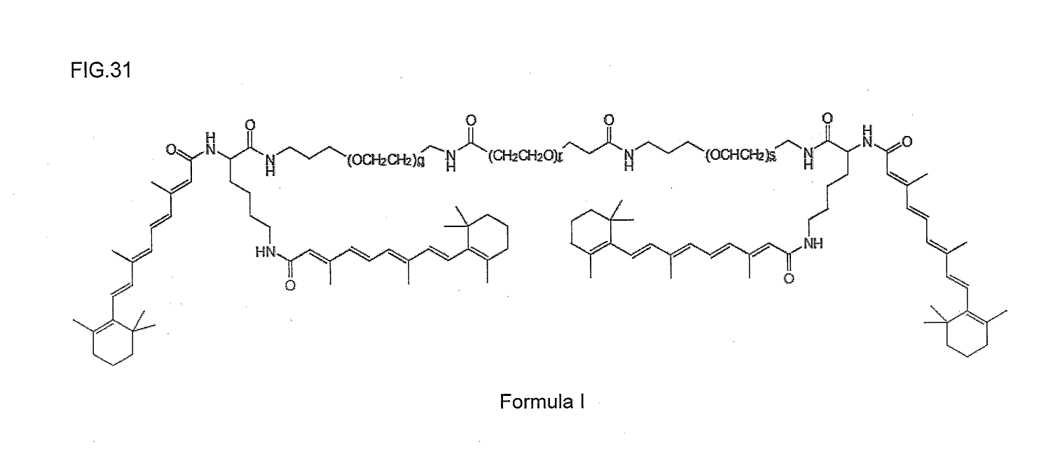

In another preferred embodiment, the compound is formula I wherein q, r, and s are each independently 1, 2, 3, 4, 5, 6, 7, 8, 9, or 10 (see FIG. 31).

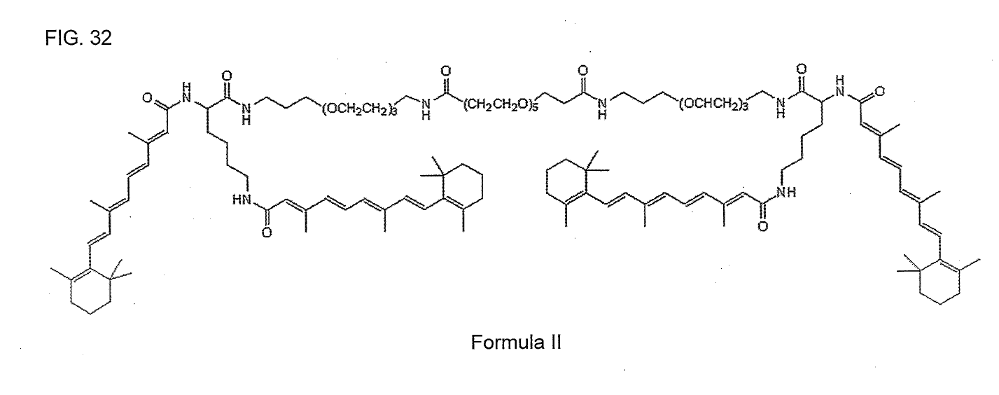

In other preferred embodiments, the compound is formula II (see FIG. 32)

Other embodiments of the invention include the structures shown in the FIGURES referred to in Table 1.

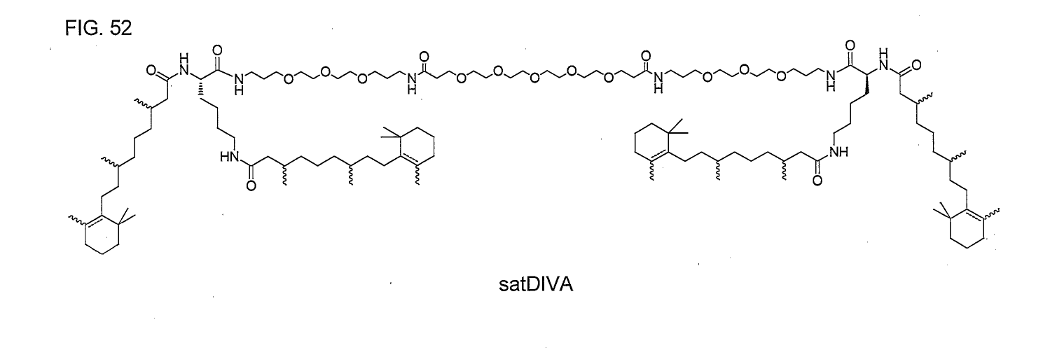

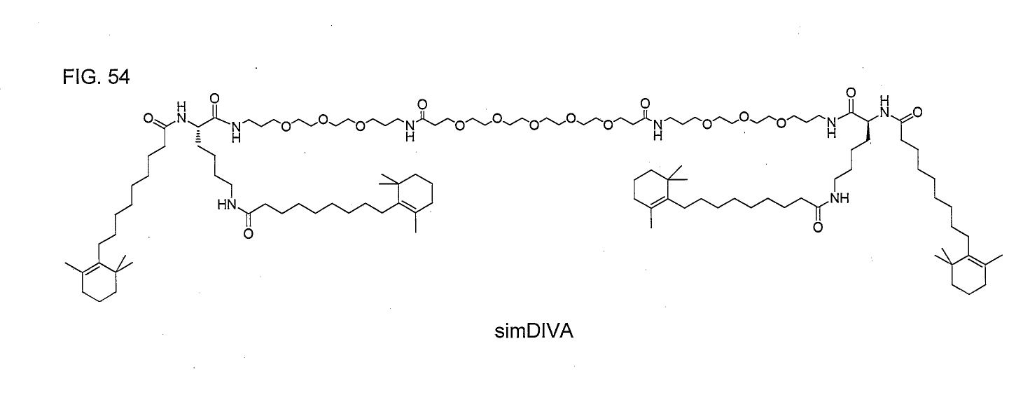

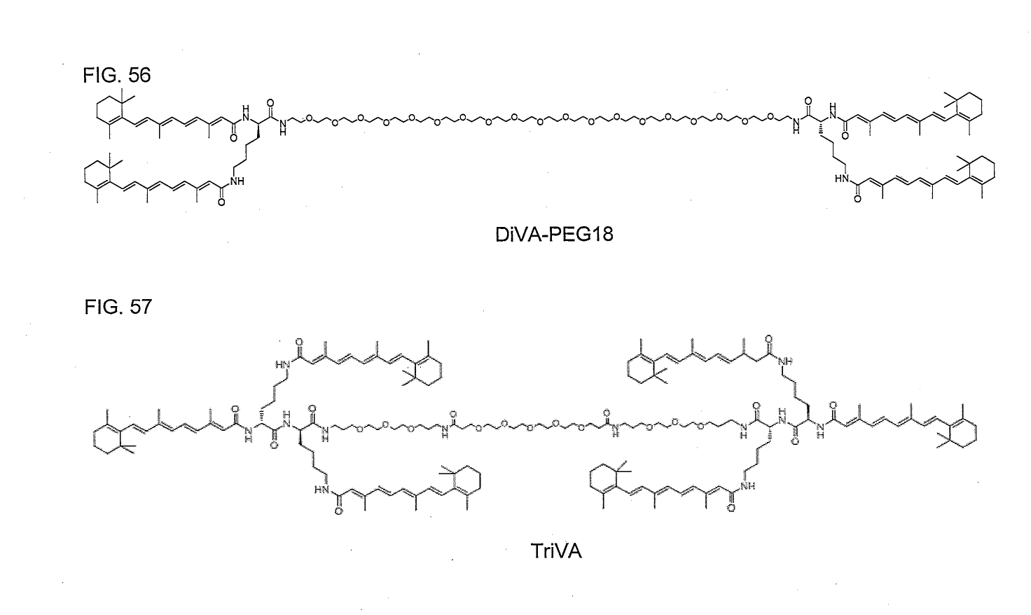

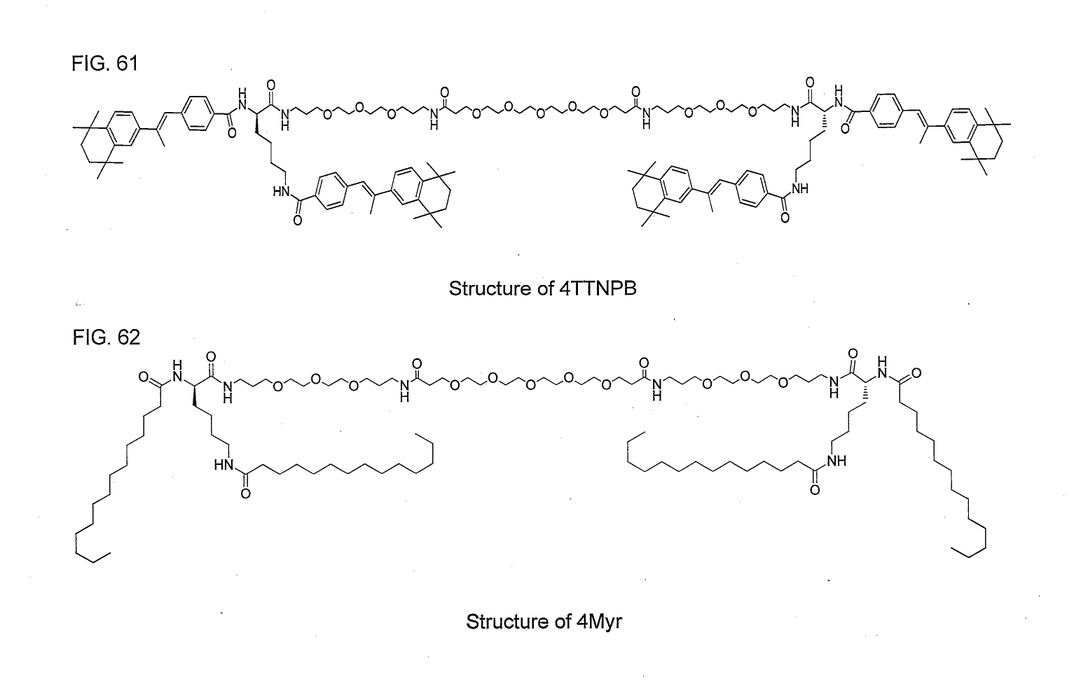

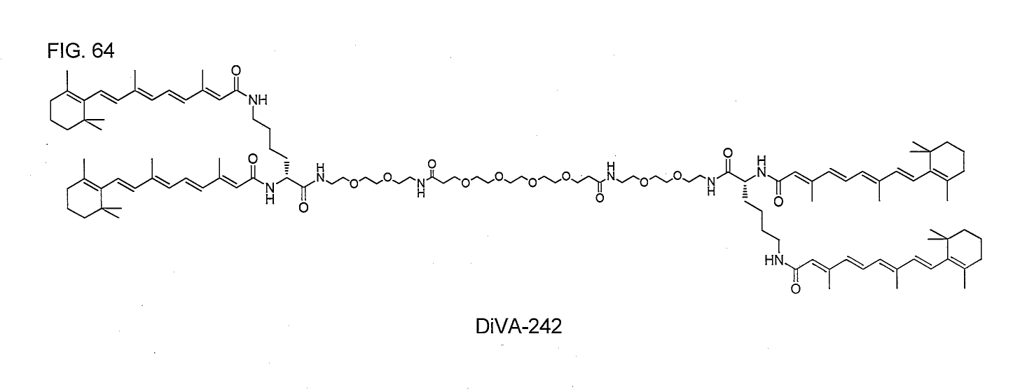

TABLE 1

| | FIG. illustrating |
|---|---|
| Lipid Name | Compound Structure |
| SatDiVA | FIG. 52 |
| SimDiVA | FIG. 54 |
| DiVA-PEG18 | FIG. 56 |
| TriVA | FIG. 57 |
| 4TTNPB | FIG. 61 |
| 4Myr | FIG. 62 |
| DiVA-242 | FIG. 64 |

Also within the scope of the invention are formulations comprising at least one compound of Formula A or B and siRNA. It is envisioned that any siRNA molecule can be used within the scope of the invention. Examples of siRNA include:

Sense
(SEQ. ID. NO. 1)
(5′->3′)GGACAGGCCUCUACAACUATT
Antisense
(SEQ. ID. NO. 2)
(3′->5′)TTCCUGUCCGGAGAUGUUGAU
and
Sense
(SEQ. ID. NO. 3)
(5′->3′)GGACAGGCCUGUACAACUATT
Antisense
(SEQ. ID. NO. 4)
(3′->5′)TTCCUGUCCGGACAUGUUGAU

Also within the scope of the invention are pharmaceutical formulations that include any of the aforementioned compounds in addition to a pharmaceutically acceptable carrier or diluent. Pharmaceutical formulations of the invention will include at least one therapeutic agent. Preferably, the therapeutic agent is an siRNA. It is envisioned that any siRNA molecule can be used within the scope of the invention. As previously described, siRNA include the sequences shown as SEQ ID NOs: 1-8.

In preferred formulations of the invention including siRNA, the siRNA is encapsulated by the liposome. In other embodiments, the siRNA can be outside of the liposome. In those embodiments, the siRNA can be complexed to the outside of the liposome.

A useful range of cationic lipid:siRNA (lipid nitrogen to siRNA phosphate ratio, “N:P”) is 0.2 to 5.0. A particularly preferred range of N:P is 1.5 to 2.5 for compositions and formulations of the description.

Preferred formulations of the invention include those comprising HEDC:S104:DOPE:Cholesterol:PEG-DMPE:DiVA-PEG-DiVA (20:20:30:25:5:2 molar ratio) and HEDC:S104:DOPE:Cholesterol:PEG-DMPE:DiVA-PEG-DiVA (20:20:30:25:5:2 molar ratio) wherein DiVA-PEG-DiVA is co-solubilized. DODC:DOPE:cholesterol:PEG-lipid:DiVA-PEG-DiVA (50:10:38:2:5 molar ratio) and DODC:DOPE:cholesterol:PEG-lipid:DiVA-PEG-DiVA formulations wherein the DiVA-PEG-DiVA is co-solubilized.

Other formulations of the invention include those comprising HEDODC:DOPE:cholesterol-PEG-lipid:DiVA-PEG-DiVA (50:10:38:2:5 molar ratio) and HEDODC:DOPE:cholesterol-PEG-lipid:DiVA-PEG-DiVA formulations wherein the DiVA-PEG-DiVA is co-solubilized.

Other preferred formulations of the invention include those comprising DC-6-14:DOPE:cholesterol: DiVA-PEG-DiVA (40:30:30:5, molar ratios) and DC-6-14:DOPE:cholesterol: DiVA-PEG-DiVA, wherein the DiVA-PEG-DiVA is co-solubilized.

Also within the scope of the invention are methods of delivering a therapeutic agent to a patient. These methods comprise providing a pharmaceutical formulation including any of the foregoing compositions and a pharmaceutically acceptable carrier or diluent; and administering the pharmaceutical formulation to the patient.

### DEFINITIONS

As used herein, “alkyl” refers to a straight or branched fully saturated (no double or triple bonds) hydrocarbon group, for example, a group having the general formula —CₙH₂ₙ₊₁. The alkyl group may have 1 to 50 carbon atoms (whenever it appears herein, a numerical range such as “1 to 50” refers to each integer in the given range; e.g., “1 to 50 carbon atoms” means that the alkyl group may consist of 1 carbon atom, 2 carbon atoms, 3 carbon atoms, etc., up to and including 50 carbon atoms, although the present definition also covers the occurrence of the term “alkyl” where no numerical range is designated). The alkyl group may also be a medium size alkyl having 1 to 30 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl and the like.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

As used herein, “halogen” refers to F, Cl, Br, and I.

As used herein, “mesylate” refers to —OSO₂CH₃.

As used herein, the term “pharmaceutical formulation” refers to a mixture of a composition disclosed herein with one or more other chemical components, such as diluents or additional pharmaceutical carriers. The pharmaceutical formulation facilitates administration of the composition to an organism. Multiple techniques of administering a pharmaceutical formulation exist in the art including, but not limited to injection and parenteral administration.

As used herein, the term “pharmaceutical carrier” refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism

As used herein, the term “diluent” refers to chemical compounds diluted in water that will dissolve the formulation of interest (e.g., the formulation that can include a compound, a retinoid, a second lipid, a stabilizing agent, and/or a therapeutic agent) as well as stabilize the biologically active form of the formulation. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of the formulation. As used herein, an “excipient” refers to an inert substance that is added to a formulation to provide, without limitation, bulk, consistency, stability, binding ability, lubrication, disintegrating ability, etc., to the composition. A “diluent” is a type of excipient.

As used herein, “therapeutic agent” refers to a compound that, upon administration to a mammal in a therapeutically effective amount, provides a therapeutic benefit to the mammal. A therapeutic agent may be referred to herein as a drug. Those skilled in the art will appreciate that the term “therapeutic agent” is not limited to drugs that have received regulatory approval. A “therapeutic agent” can be operatively associated with a compound as described herein, a retinoid, and/or a second lipid. For example, a second lipid as described herein can form a liposome, and the therapeutic agent can be operatively associated with the liposome, e.g., as described herein.

As used herein, “lipoplex formulations” refer to those formulations wherein the siRNA is outside of the liposome. In preferred lipoplex formulations, the siRNA is complexed to the outside of the liposome. Other preferred lipoplex formulations include those wherein the siRNA is accessible to any medium present outside of the liposome.

As used herein, “liposome formulations” refer to those formulations wherein the siRNA is encapsulated within the liposome. In preferred liposome formulations, the siRNA is inaccessible to any medium present outside of the liposome.

As used herein, the term “co-solubilized” refers to the addition of a component to the cationic lipid mixture before the empty vesicle is formed.

As used herein, the term “decorated” refers to the addition of a component after vesicle formation.

As used herein, “DC-6-14” refers to the following cationic lipid compound:

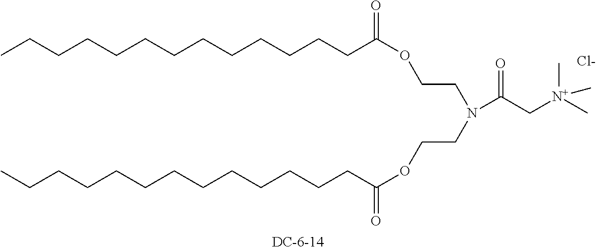 [SMILES: CCCCCCCCCCCCCC(=O)OCCN(CCOC(=O)CCCCCCCCCCCCC)C(=O)C[N+](C)(C)C ; [Cl-]]

As used herein, “DODC” refers to the following cationic lipid compound:

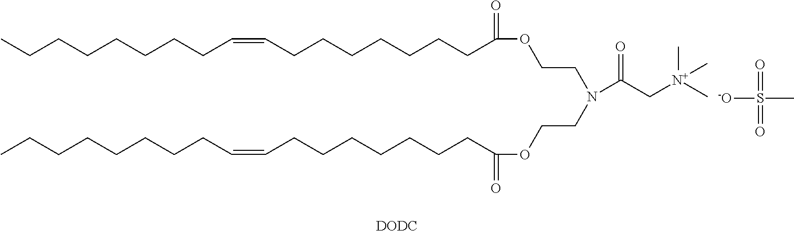

As used herein, “HEDODC” refers to the following cationic lipid compound:

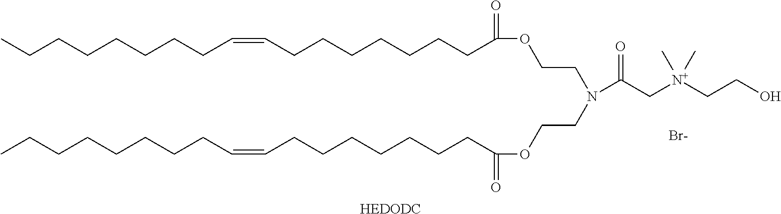 [SMILES: CCCCCCCC/C=C\CCCCCCCC(=O)OCCN(CCOC(=O)CCCCCCC/C=C\CCCCCCCC)C(=O)C[N+](C)(C)CCO ; [Br-] ; "HEDODC"]

As used herein, a “retinoid” is a member of the class of compounds consisting of four isoprenoid units joined in a head-to-tail manner, see G. P. Moss, “Biochemical Nomenclature and Related Documents,” 2nd Ed. Portland Press, pp. 247-251 (1992). “Vitamin A” is the generic descriptor for retinoids exhibiting qualitatively the biological activity of retinol. As used herein, retinoid refers to natural and synthetic retinoids including first generation, second generation, and third generation retinoids. Examples of naturally occurring retinoids include, but are not limited to, (1) 11-cis-retinal, (2) all-trans retinol, (3) retinyl palmitate, (4) all-trans retinoic acid, and (5) 13-cis-retinoic acids. Furthermore, the term “retinoid” encompasses retinols, retinals, retinoic acids, rexinoids, demethylated and/or saturated retinoic acids, and derivatives thereof.

As used herein, “Vitamin D” is a generic descriptor for a group of vitamins having antirachitic activity. The vitamin D group includes: vitamin D₂ (calciferol), vitamin D₃ (irradiated 22-dihydroergosterol), vitamin D₄ (irradiated dehydrositosterol) and vitamin D₅ (irradiated dehydrositosterol).

As used herein, “Vitamin E” is a generic descriptor for a group of molecules with antioxidant activity. The vitamin E family includes α-tocopherol, β-tocopherol, γ-tocopherol and δ-tocopherol, with α-tocopherol being the most prevalent. (Brigelius-Flohe and Traber, The FASEB Journal. 1999;13:1145-1155).

As used herein, “Vitamin K” is generic descriptor for an antihemorrahgic factor and includes vitamin K₁ (phytonodione), vitamin K₂ (menaquinone), vitamin K₃, vitamin K₄ and vitamin K₅. Vitamins K₁ and K₂ are natural, while K3-5 are synthetic.

As used herein, “retinoid-linker-lipid molecule” refers to a molecule that includes at least one retinoid moiety attached to at least one lipid moiety through at least one linker such as, for example, a PEG moiety.

As used herein, “retinoid-linker-retinoid molecule” refers to a molecule that includes at least one retinoid moiety attached to at least one other retinoid moiety (which may be the same or different) through at least one linker such as, for example, a PEG moiety.

As used herein, the terms “lipid” and “lipophilic” are used herein in their ordinary meanings as understood by those skilled in the art. Non-limiting examples of lipids and lipophilic groups include fatty acids, sterols, C₂-C₅₀ alkyl, C₂-C₅₀ heteroalkyl, C₂-C₅₀ alkenyl, C₂-C₅₀ heteroalkenyl, C₅-C₅₀ aryl, C₅-C₅₀ heteroaryl, C₂-C₅₀ alkynyl, C₂-C₅₀ heteroalkynyl, C₂-C₅₀ carboxyalkenyl, and C₂-C₅₀ carboxyheteroalkenyl. A fatty acid is a saturated or unsaturated long-chain monocarboxylic acid that contains, for example, 12 to 24 carbon atoms A lipid is characterized as being essentially water insoluble, having a solubility in water of less than about 0.01% (weight basis). As used herein, the terms “lipid moiety” and “lipophilic moiety” refers to a lipid or portion thereof that has become attached to another group. For example, a lipid group may become attached to another compound (e.g., a monomer) by a chemical reaction between a functional group (such as a carboxylic acid group) of the lipid and an appropriate functional group of a monomer.

As used herein, “siRNA” refers to small interfering RNA, also known in the art as short interfering RNA or silencing RNA. siRNA is a class of double stranded RNA molecules that have a variety of effects known in the art, the most notable being the interference with the expression of specific genes and protein expression.

As used herein, “encapsulated by the liposome” refers to a component being substantially or entirely within the liposome structure.

As used herein, “accessible to the aqueous medium” refers to a component being able to be in contact with the aqueous medium.

As used herein, “inaccessible to the aqueous medium” refers to a component not being able to be in contact with the aqueous medium.

As used herein, “complexed on the outer surface of the liposome” refers to refers to a component being operatively associated with the outer surface of the liposome.

As used herein, “localized on the outer surface of the liposome” refers to a component being at or near the outer surface of the liposome.

As used herein, “charge complexed” refers to an electrostatic association.

As used herein, the term “operatively associated” refers to an electronic interaction between a compound as described herein, a therapeutic agent, a retinoid, and/or a second lipid. Such interaction may take the form of a chemical bond, including, but not limited to, a covalent bond, a polar covalent bond, an ionic bond, an electrostatic association, a coordinate covalent bond, an aromatic bond, a hydrogen bond, a dipole, or a van der Waals interaction. Those of ordinary skill in the art understand that the relative strengths of such interactions may vary widely.

The term “liposome” is used herein in its ordinary meaning as understood by those skilled in the art, and refers to a lipid bilayer structure that contains lipids attached to polar, hydrophilic groups which form a substantially closed structure in aqueous media. In some embodiments, the liposome can be operatively associated with one or more compounds, such as a therapeutic agent and a retinoid or retinoid conjugate. A liposome may be comprised of a single lipid bilayer (i.e., unilamellar) or it may comprised of two or more concentric lipid bilayers (i.e., multilamellar). Additionally, a liposome can be approximately spherical or ellipsoidal in shape.

The term “facilitating drug delivery to a target cell” refers the enhanced ability of the present retinoid or fat soluble vitamin compounds to enhance delivery of a therapeutic molecule such as siRNA to a cell. While not intending to be bound by theory, the retinoid or fat-soluble vitamin compound interacts with a specific receptor (or activation/binding site) on a target cell with specificity that can be measured. For example, binding is generally consider specific when binding affinity (Kₐ) of 10⁶ M⁻¹ or greater, preferably 10⁷ M⁻¹ or greater, more preferably 10⁸M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660, 1949).

Also within the scope of the invention is a composition, containing a collagen-reducing substance, for regenerating normal tissue from fibrotic tissue.

In the present invention, a ‘collagen-reducing substance’ means any substance that can reduce the amount of collagen accumulated in tissue. Although it is not intended to be bound by a specific theory, since one of the causes for the accumulation of collagen in fibrotic tissue is thought to be a shift in the balance between production and decomposition of collagen to the production side, the collagen-reducing substance can include not only a suppressor of collagen production, but also a collagen decomposition promoter and a suppressor of an inhibitor of a collagen decomposition promoter. Therefore, examples of the collagen-reducing substance include, but are not limited to, a suppressor of collagen production by collagen-producing cells, a promoter of collagen decomposition, and a suppressor of a collagen decomposition inhibitor. Although there is no particular limitation, the collagen in the present invention is preferably a collagen involved in fibrosis such as for example type I, III, or V collagen, and particularly preferably type I collagen, which is present in fibrotic tissue in the largest amount.

In the present invention, the collagen-producing cells mean any cells that produce collagen in fibrotic tissue, and examples include, but are not limited to, activated stellate cells and myofibroblasts. It is thought that activated stellate cells and myofibroblasts are the main collagen-producing sources in fibrotic tissue, and they are characterized by the expression of α-SMA (α-smooth muscle actin). Therefore, the activated stellate cells and myofibroblasts in the present invention are identified by means of immunostaining, etc. using an anti-α-SMA antibody that is detectably labeled.

The suppressor of collagen production by collagen-producing cells includes any drug that directly or indirectly suppresses the physical, chemical, and/or physiological actions, etc. of same cells involved in collagen accumulation in fibrotic tissue, and examples thereof include, but are not limited to, a TGFβ (Transforming growth factor-beta) inhibitor, HGF (Hepatocyte growth factor) or a substance promoting the production thereof, a PPARγ (Peroxisome proliferator-activated receptor gamma) ligand, an angiotensin inhibitor, a PDGF (Platelet-derived growth factor) inhibitor, relaxin or a substance promoting the production thereof, a substance that inhibits the production and secretion of an extracellular matrix component, a cell activity suppressor, a cell growth suppressor, and an apoptosis-inducing substance.

Examples of the TGFβ inhibitor include, but are not limited to, a truncated TGFβ type II receptor (Qi et al., Proc Natl Acad Sci USA. 1999; 96 (5): 2345-9), a soluble TGFβ type II receptor (George et al., Proc Natl Acad Sci USA. 1999; 96 (22): 12719-24), a TGFβ activity inhibitor such as an anti-TGFβ antibody, a TGFβ production inhibitor such as an RNAi molecule, ribozyme, or antisense nucleic acid complementary to TGFβ, vectors expressing these, and cells transformed thereby. In one embodiment of the present invention, the TGFβ inhibitor inhibits the activity and/or production of TGFβ1.

Examples of substances promoting the production of HGF or relaxin include, but are not limited to, a nucleic acid coding for HGF or relaxin, an expression construct containing this, expression vectors containing these, and cells transformed thereby.

Examples of the PPARγ ligand include, but are not limited to, an endogenous ligand such as 15-deoxy-Δ12,14-prostaglandin J2, nitrolinoleic acid, oxidized LDL (Low density lipoprotein), a long chain fatty acid, or an eicosanoid, and an exogenous ligand such as a thiazolidinedione medicinal agent such as troglitazone, pioglitazone, rosiglitazone, balaglitazone or rivoglitazone, or a non-steroidal anti-inflammatory drug.

Examples of the angiotensin inhibitor include, but are not limited to, an angiotensin receptor antagonist such as telmisartan, losartan, valsartan, candesartan cilexetil, olmesartan medoxomil, or irbesartan. The angiotensin includes angiotensins I, II, III, and IV. Furthermore, examples of the angiotensin receptor include, but are not limited to, an angiotensin type 1 receptor (ATI).

Examples of the PDGF inhibitor include, but are not limited to, a PDGF activity inhibitor such as an anti-PDGF antibody, a PDGF production inhibitor such as an RNAi molecule, ribozyme, or antisense nucleic acid complementary to PDGF, vectors expressing these, and cells transformed thereby.

Examples of the substance that inhibits the production and secretion of an extracellular matrix component include, but are not limited to, a substance, such as an RNAi molecule, a ribozyme, or an antisense nucleic acid, that suppresses the expression of an extracellular matrix component such as collagen, proteoglycan, tenascin, fibronectin, thrombospondin, osteopontin, osteonectin, or elastin, a substance having a dominant negative effect such as a dominant negative mutant, vectors expressing these, and cells transformed thereby. Examples of drugs that inhibit the production and secretion of collagen include, but are not limited to, inhibitors of HSP (Heat shock protein) 47, which is a collagen-specific molecular chaperone essential for intracellular transport and molecular maturation common to the synthetic processes for various types of collagen, for example HSP47 expression inhibitors such as an RNAi molecule, ribozyme, or antisense nucleic acid complementary to HSP47, a substance having a dominant negative effect such as an HSP47 dominant negative mutant, vectors expressing these, and cells transformed thereby.

Examples of the cell growth suppressor include, but are not limited to, an alkylating agent (e.g. ifosfamide, nimustine, cyclophosphamide, dacarbazine, melphalan, ranimustine, etc.), an antitumor antibiotic (e.g. idarubicin, epirubicin, daunorubicin, doxorubicin, pirarubicin, bleomycin, peplomycin, mitoxantrone, mitomycin C, etc.), a metabolism antagonist (e.g. gemcitabine, enocitabine, cytarabine, tegafur-uracil, tegafur-gimeracil-oteracil potassium combination drug, doxifluridine, hydroxycarbamide, fluorouracil, methotrexate, mercaptopurine, etc.), an alkaloid such as etoposide, irinotecan, vinorelbine, docetaxel, paclitaxel, vincristine, vindesine, or vinblastine, a platinum complex such as carboplatin, cisplatin, or nedaplatin, and a statin such as lovastatin or simvastatin.

Examples of the cell activity suppressor include, but are not limited to, a sodium channel inhibitor.

Examples of the apoptosis-inducing agent include, but are not limited to, compound 861, gliotoxin, and atorvastatin.

Examples of the promoter of collagen decomposition include, but are not limited to, various types of collagenase and a substance promoting the production thereof. Examples of the collagenase include, but are not limited to, the MMP family, such as MMP (Matrix metalloproteinase) 1, 2, 3, 9, 13, and 14. Examples of the collagenase production promoter include, but are not limited to, a nucleic acid coding for the collagenase, an expression construct containing this, expression vectors containing these, and cells transformed thereby.

Examples of the inhibitor of a collagen decomposition promoter include, but are not limited to, TIMP (Tissue inhibitor of metalloproteinase, TIMP1 and TIMP2, etc.). Therefore, examples of the suppressor of the above inhibitor include, but are not limited to, a TIMP activity inhibitor such as an antibody for TIMP, a TIMP production inhibitor such as an RNAi molecule, ribozyme, or antisense nucleic acid complementary to TIMP, vectors expressing these, and cells transformed thereby.

The RNAi molecule in the present invention includes RNA such as siRNA (small interfering RNA), miRNA (micro RNA), shRNA (short hairpin RNA), ddRNA (DNA-directed RNA), piRNA (Piwi-interacting RNA), rasiRNA (repeat associated siRNA), and modifications of these. Furthermore, the nucleic acid in the present invention includes RNA, DNA, PNA, and composites thereof.

In the present invention, ‘fibrotic tissue’ means tissue in which extracellular matrix, mainly collagen, has accumulated in an amount greater than normal. In addition to collagen, examples of the extracellular matrix include, but are not limited to, proteoglycan, tenascin, fibronectin, thrombospondin, osteopontin, osteonectin, and elastin. The amount of collagen accumulated in tissue may be quantified for example by using the amount of hydroxyproline in the tissue as an indicator or by subjecting the tissue to collagen staining (e.g. Masson trichrome staining, Azan staining, sirius red staining, Elastica van Gieson staining, etc.) and carrying out an image analysis. The amount of extracellular matrix in fibrotic tissue in the present invention may be at least 5%, at least 10%, at least 25%, at least 50%, at least 100%, at least 200%, at least 300%, at least 400%, or at least 500% compared with that of normal tissue. Since it is thought that the production of collagen by activated stellate cells and/or myofibroblasts contributes to fibrosis of tissue, the fibrotic tissue in the present invention typically contains activated stellate cells and/or myofibroblasts. The fibrotic tissue may be any tissue in the body as long as it has the above-mentioned features, and examples thereof include, but are not limited to, the liver, the pancreas, the lung, the kidney, the bone marrow, the vocal cord, the larynx, the mouth cavity, the heart, the spleen, the mediastinum, the retroperitoneum, the uterus, the skin, the mammary gland, and the intestinal tract.

Therefore, the fibrotic tissue may be an affected area in various organ fibroses. Examples of the organ fibroses include, but are not limited to, hepatic fibrosis, hepatic cirrhosis, vocal cord scar formation, vocal cord mucosal fibrosis, laryngeal fibrosis, pulmonary fibrosis, pancreatic fibrosis, myelofibrosis, myocardial infarction, fibrosis of the myocardium following myocardial infarction, myocardial fibrosis, endomyocardial fibrosis, splenic fibrosis, mediastinal fibrosis, lingual submucous fibrosis, intestinal fibrosis (e.g. that associated with an inflammatory bowel disease, etc.), retroperitoneal fibrosis, uterine fibrosis, scleroderma, and a fibrous disease of the breast.

The hepatic fibrosis and hepatic cirrhosis in the present invention include not only those caused by a viral infection with hepatitis B or C virus, drinking alcohol, fatty liver, a parasitic infection, a congenital metabolic abnormality, a hepatotoxic substance, etc., but also those for which the cause is not specified. Therefore, examples of the hepatic cirrhosis in the present invention include, but are not limited to, Charcot's cirrhosis, Todd's cirrhosis, primary biliary cirrhosis, unilobar cirrhosis, secondary biliary cirrhosis, obstructive cirrhosis, cholangiolitic cirrhosis, biliary cirrhosis, atrophic cirrhosis, nutritional cirrhosis, postnecrotic cirrhosis, posthepatitic cirrhosis, nodular cirrhosis, mixed cirrhosis, micronodular cirrhosis, compensated cirrhosis, macronodular cirrhosis, septal cirrhosis, cryptogenic cirrhosis, decompensated cirrhosis, periportal cirrhosis, portal cirrhosis, and alcoholic cirrhosis.

The pulmonary fibrosis in the present invention includes not only pulmonary fibrosis in a strict sense but also pulmonary fibrosis in a broad sense, including coexistence with interstitial pneumonia. The pulmonary fibrosis in the present invention can be caused by any interstitial pneumonia such as for example infectious interstitial pneumonia associated with viral pneumonia, fungal pneumonia, mycoplasma pneumonia, etc., interstitial pneumonia associated with a collagen disease such as rheumatoid arthritis, systemic scleroderma, dermatomyositis, polymyositis, a mixed connective tissue disease (MCTD, Mixed connective tissue disease), interstitial pneumonia associated with radiation exposure, interstitial pneumonia induced by a drug such as an anticancer agent such as bleomycin, a Chinese herbal medicine such as Sho-saiko-to, interferon, an antibiotic, or Paraquat, or idiopathic interstitial pneumonia such as idiopathic pulmonary fibrosis, nonspecific interstitial pneumonia, acute interstitial pneumonia, cryptogenic organizing pneumonia, a respiratory bronchiolitis-associated interstitial lung disease, desquamating interstitial pneumonia, or lymphocytic interstitial pneumonia, and the pulmonary fibrosis in the present invention therefore includes those in which the above interstitial pneumonia has become chronic.

The myelofibrosis in the present invention includes not only primary myelofibrosis but also secondary myelofibrosis. Examples of the secondary myelofibrosis include, but are not limited to, those that are secondary to a disease such as acute myeloid leukemia, acute lymphoblastic leukemia, chronic myeloid leukemia, polycythemia vera, primary thrombocythemia, myelodysplastic syndrome, multiple myeloma, malignant lymphoma, carcinoma, systemic lupus erythematosus, or progressive systemic sclerosis, or to radiation exposure.

Renal fibrosis in the present invention can be caused by any interstitial nephritis such as for example infectious interstitial nephritis associated with streptococcal nephritis, staphylococcal nephritis, pneumococcal nephritis, viral nephritis associated with varicella, hepatitis B, hepatitis C, HIV, etc., nephritis due to a parasitic infection such as malaria, fungal nephritis, mycoplasma nephritis, etc., interstitial nephritis associated with a collagen disease such as systemic lupus erythematosus (lupus nephritis), systemic scleroderma (collagen disease of the kidney), or Sjogren syndrome, nephritis associated with a blood vessel immune disease such as purpura nephritis, polyarteritis, rapidly progressive glomerulonephritis, etc., interstitial nephritis associated with radiation exposure, interstitial nephritis induced by a drug such as a gold drug, an NSAID, penicillamine, an anticancer agent such as bleomycin, an antibiotic, or Paraquat, etc., an allergic nephritis due to an insect bite, pollen, or an Anacardiaceae family plant, amyloidosis nephritis, diabetic nephropathy, chronic glomerulonephritis, nephritis associated with malignant nephrosclerosis or a polycystic kidney disease, tubulointerstitial nephritis, nephritis associated with gestational toxicosis or a cancer, membranoproliferative glomerulonephritis, IgA nephropathy nephritis, mixed cryoglobulinemic nephritis, Goodpasture's syndrome nephritis, Wegener's granulomatous nephritis, or an idiopathic interstitial nephritis such as acute interstitial nephritis, etc., and the renal fibrosis in the present invention therefore includes those in which the above interstitial nephritis has become chronic.

In one embodiment of the present invention, the fibrotic tissue is that which continually receives a fibrotic stimulus. In the present invention, the fibrotic stimulus means any stimulus that induces fibrosis, and examples include, but are not limited to, oxidative stress, hypoxia, inflammation, and apoptosis (see Ghiassi-Nejad et al., Expert Rev Gastroenterol Hepatol. 2008; 2(6): 803-16). Examples of such tissue include fibrotic tissue that is experiencing chronic inflammation and tissue that is continuously exposed to a cytotoxic substance (e.g. liver tissue in which cholestasis is caused by a bile duct disease, etc.). Furthermore, such tissue also includes tissue affected by fibrosis for which the direct cause of the disease is unclear, such as for example cryptogenic cirrhosis, idiopathic pulmonary fibrosis, or idiopathic myelofibrosis, etc., or affected by those for which the direct cause of the disease is known but the origin of the cause of the disease is unclear or it is difficult to remove, such as for example primary biliary cirrhosis, nonalcoholic steatohepatitis (NASH)-derived hepatic fibrosis, primary sclerosing cholangitis, idiopathic pulmonary fibrosis, idiopathic interstitial pneumonia-derived pulmonary fibrosis, primary myelofibrosis, idiopathic interstitial nephritis-derived renal fibrosis, inflammatory bowel disease (e.g. Crohn's disease, ulcerative colitis, etc.), or systemic scleroderma, etc.

In the present invention, ‘regenerating normal tissue from fibrotic tissue’ means recovering the tissue that has been denatured due to fibrosis at least to a state in which the fibrosis is of a lesser degree. That is, as fibrosis progresses, tissue is replaced by fibrous tissue, which is mainly extracellular matrix, and the regeneration of normal tissue from fibrotic tissue in the present invention is to reverse the above flow and replace the proliferated fibrous tissue with the original normal tissue. Therefore, the regeneration of normal tissue from fibrotic tissue in the present invention includes not only completely recovering fibrotic tissue to the original state but also partially recovering fibrotic tissue to the original state. The degree of regeneration of normal tissue may be evaluated by a histological examination of a biopsy sample, etc. based on normalization of the tissue structure, reduction in the region occupied by fibrous tissue, increase in the region occupied by normal tissue, etc., or when an abnormality of a biochemical index due to fibrosis is observed before treatment with the present composition, evaluation may be carried out based on improvement of the index, etc.

In one embodiment of the present invention, regeneration of normal tissue may be carried out by growth and differentiation of stem cells in a space that is formed due to reduction of collagen accumulated in fibrotic tissue. Therefore, one embodiment of the present invention relates to the pharmaceutical composition wherein it is for regenerating normal tissue from fibrotic tissue in a space for the growth and differentiation of stem cells, the space being formed by a reduction of collagen accumulated in the fibrotic tissue. Here, examples of the stem cells include, but are not limited to, those that are originally present in the tissue that has become fibrotic (hepatic stem cells, pancreatic stem cells, lung stem cells, renal stem cells, bone marrow stem cells, heart stem cells, spleen stem cells, uterine stem cells, skin stem cells, mammary stem cells, intestinal stem cells, mesenchymal stem cells, etc.), those that have moved from another place in the body and, furthermore, those that have been therapeutically administered. Moreover, the ‘space’ includes not only a cavity within the tissue but also a space with room in which cells can enlarge and grow such as for example a space in which the pressure between cells is decreased or a space having flexibility.

In one embodiment, the composition of the present invention further contains a targeting agent for collagen-producing cells in fibrotic tissue. By containing the targeting agent, it becomes possible to specifically deliver to collagen-producing cells, which are target cells, a collagen-reducing substance that is targeted to collagen-producing cells such as, for example, without limitation, a substance that inhibits the production and secretion of an extracellular matrix component, HGF or a substance promoting the production thereof, MMP or a substance promoting the production thereof, a TIMP inhibitor, a TGFβ production inhibitor, relaxin or a substance promoting the production thereof, etc., thereby enhancing the effect of the collagen-reducing substance used.

In one embodiment of the present invention, the targeting agent for collagen-producing cells is a retinoid. Although the mechanism in which targeting is carried out by means of a retinoid has not yet been clarified, it is surmised for example that a retinoid bound specifically to an RBP (Retinol binding protein) is incorporated into a collagen-producing cell in fibrotic tissue via a certain type of receptor positioned on the surface of the cell. The ability of a retinoid to function as a targeting agent for collagen-producing cells is described in WO 2006/068232, JP, A, 2009-221164, JP, A, 2010-59124, etc.

A retinoid is one member of a group of compounds having a skeleton in which four isoprenoid units are connected in a head-to-tail manner (see G. P. Moss, “Biochemical Nomenclature and Related Documents”, 2nd Ed. Portland Press, pp. 247-251 (1992)), and vitamin A is a generic descriptor for a retinoid qualitatively showing the biological activity of retinol. Examples of the retinoid that can be used in the present invention include, but are not particularly limited to, retinol (including all-trans retinol), retinal, retinoic acid (including tretinoin), an ester of retinol and a fatty acid, an ester of an aliphatic alcohol and retinoic acid, a retinoid derivative such as etretinate, isotretinoin, adapalene, acitretin, tazarotene, or retinyl palmitate, and a vitamin A analog such as fenretinide (4-HPR) or bexarotene.

Among them, retinol, retinal, retinoic acid, an ester of retinol and a fatty acid (e.g. retinyl acetate, retinyl palmitate, retinyl stearate, and retinyl laurate, etc.), and an ester of an aliphatic alcohol and retinoic acid (e.g. ethyl retinoate, etc.) are preferable in terms of efficiency of specific delivery of a substance to collagen-producing cells in fibrotic tissue.

All isomers, including cis/trans retinoids, are included in the scope of the present invention. A retinoid can be substituted with one or more substituents. The retinoid in the present invention includes not only one in an isolated state as well as a retinoid in a state in which it is dissolved or mixed in a medium that can dissolve or retain same. The retinoid may be provided as a compound containing one or more retinoid moieties, such as the compound of Formula A wherein the targeting molecule is a retinoid or the compound of Formula B wherein the targeting molecule is a retinoid.

The above-mentioned embodiment of the composition of the present invention may be formed only from a collagen-reducing substance targeted to collagen-producing cells as an active ingredient and a retinoid as a targeting agent, or may contain a carrier-constituting component other than the above. The carrier-constituting component in the present embodiment is not particularly limited; any component that is known in the medicinal and/or pharmaceutical fields may be used, but one for which at least inclusion of a retinoid or binding thereto is possible is preferable.

Examples of such a component include, but are not limited to, a lipid, for example, a phospholipid such as a glycerophospholipid, a sphingolipid such as sphingomyelin, a sterol such as cholesterol, a plant oil such as soybean oil or poppy seed oil, a mineral oil, a lecithin such as egg yolk lecithin, and a polymer. Among them, one that can form a liposome, such as for example a natural phospholipid such as lecithin, a semisynthetic phospholipid such as dimyristoylphosphatidylcholine (DMPC), dipalmitoylphosphatidylcholine (DPPC), or distearoylphosphatidylcholine (DSPC), dioleylphosphatidylethanolamine (DOPE), dilauroylphosphatidylcholine (DLPC), or cholesterol is preferable.

A component that can avoid capture by the reticuloendothelial system is particularly preferred, and examples thereof include cationic lipids such as N-(α-trimethylammonioacetyl)-didodecyl-D-glutamate chloride (TMAG), N,N′,N″,N′″-tetramethyl-N,N′,N″,N′″--tetrapalmitylspermine (TMTPS), 2,3-dioleyloxy-N-[2(sperminecarboxamide)ethyl]-N,N-dimethyl-1-propanaminium trifluoroacetate (DOSPA), N-[1-(2,3-dioleyloxy)propyl]-N,N,N-trimethylammonium chloride (DOTMA), dioctadecyldimethylammonium chloride (DODAC), didodecylammonium bromide (DDAB), 1,2-dioleyloxy-3-trimethylammoniopropane (DOTAP), 3β-[N-(N′,N′-dimethylaminoethane)carbamoyl]cholesterol (DC-Chol), 1,2-dimyristoyloxypropyl-3-dimethylhydroxyethylammonium bromide (DMRIE), and O,O′-ditetradecanoyl-N-(α-trimethylammonioacetyl)diethanolamine chloride (DC-6-14).

The above carrier may have a specific 3-dimensional structure. Examples of such a structure include, but are not limited to, a straight-chain or branched linear structure, a film-like structure, and a spherical structure. Therefore, the carrier may have, without limitation, any 3-dimensional form such as a micelle, a liposome, an emulsion, a microsphere, or a nanosphere.

Binding of a retinoid and/or an active ingredient to a carrier or the inclusion thereof in a carrier may also be possible by binding the retinoid to a carrier or the inclusion thereof in a carrier by means of a chemical and/or physical method. Alternatively, binding of a retinoid and/or an active ingredient to a carrier or the inclusion thereof in a carrier may also be possible by mixing a retinoid and/or an active ingredient and a carrier-constituting component. The amount of retinoid in the composition of the present invention may be for example 0.01 to 1000 nmol/μL, and preferably 0.1 to 100 nmol/μL. Furthermore, the amount of active ingredient in the composition of the present invention may be for example 1 to 10000 ng/μL, and preferably 10 to 1000 ng/μL, or 1 to 1000000 μg/kg body weight, and preferably 10 to 100000 μg/kg body weight. The amounts of retinoid and active ingredient might, in some cases, be outside the above ranges depending on the activity of these components, the administration route of the composition, the administration frequency, the subject to which they are administered, etc., and these cases are also included in the scope of the present invention. Binding of a retinoid and/or an active ingredient to a carrier or the inclusion thereof in a carrier may be carried out prior to supporting an active ingredient on the carrier, may be carried out by simultaneously mixing a carrier-constituting component, a retinoid, and an active ingredient, or may be carried out by mixing a carrier having an active ingredient already supported thereon and a retinoid. Therefore, the present invention also relates to a method for producing a pharmaceutical composition for regenerating normal tissue from fibrotic tissue that includes a step of binding a retinoid to any existing drug-binding carrier or drug-encapsulating carrier, for example, a liposome preparation such as DaunoXome®, Doxil, Caelyx®, or Myocet®.

The composition of the present invention may be in any form as long as a desired active ingredient can be transported to collagen-producing cells in fibrotic tissue as a target, and examples thereof include, but are not limited to, a polymer micelle, a liposome, an emulsion, a microsphere, and a nanosphere. In the present invention, from the viewpoint of high efficiency of delivery, wide choice of substances to be delivered, ease of preparation, etc., among the above a liposome form (i.e., liposomal composition) is preferable, and a cationic liposome that contains a cationic lipid is particularly preferable. When the composition is in the form of a liposome, the molar ratio of retinoid and liposome-constituting lipid is preferably 8:1 to 1:4, and more preferably 4:1 to 1:2, while taking into consideration the efficiency of binding of a retinoid to a carrier or the inclusion thereof in a carrier.

The liposomal composition can comprise a lipid vesicle comprising a bilayer of lipid molecules. In certain embodiments it may preferred that the retinoid molecule is at least partially exposed on the exterior of the drug carrier before the drug carrier reaches the target cell.

Certain embodiments of the present invention provide that the lipid molecules comprise one or more lipids selected from the group consisting of HEDC, DODC, HEDODC, DSPE, DOPE, and DC-6-14. In other embodiments, the lipid molecules can further comprise S104.

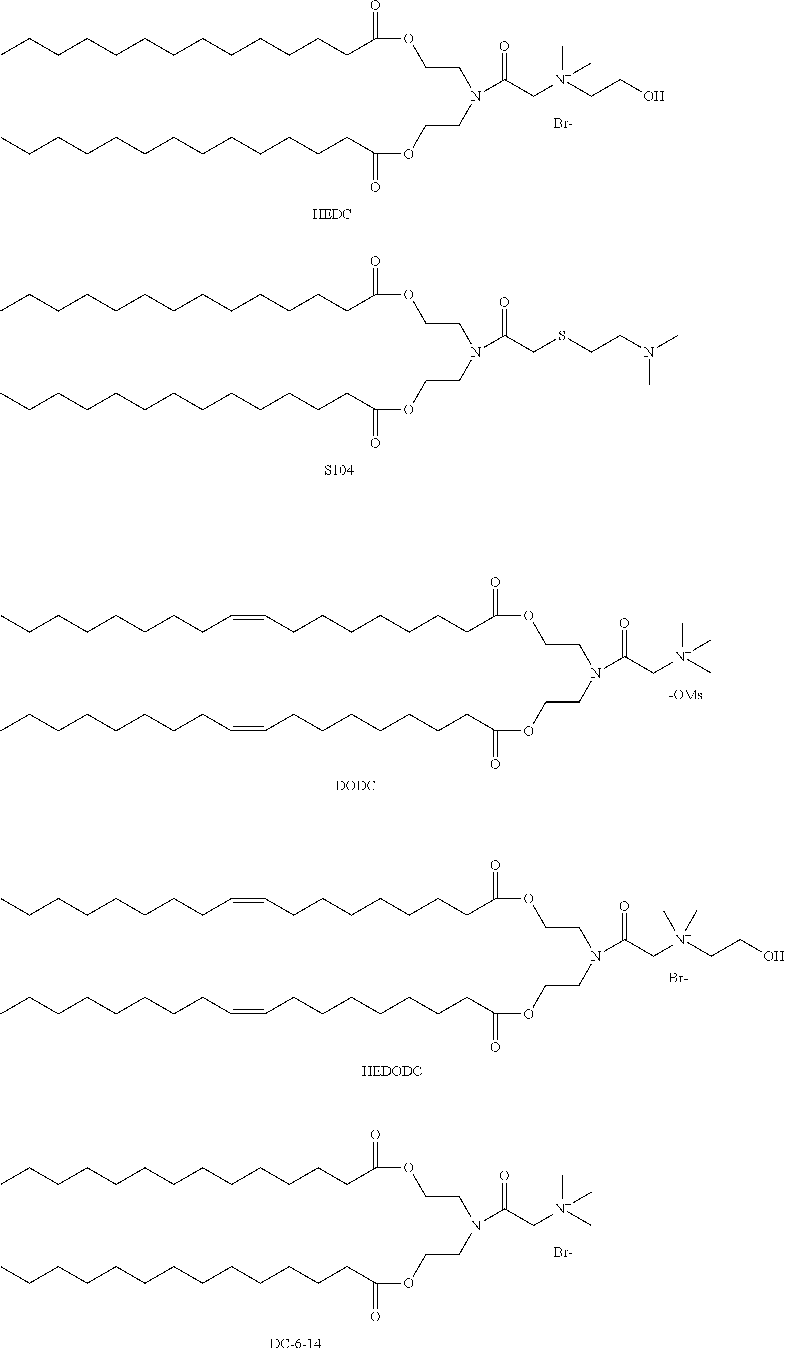

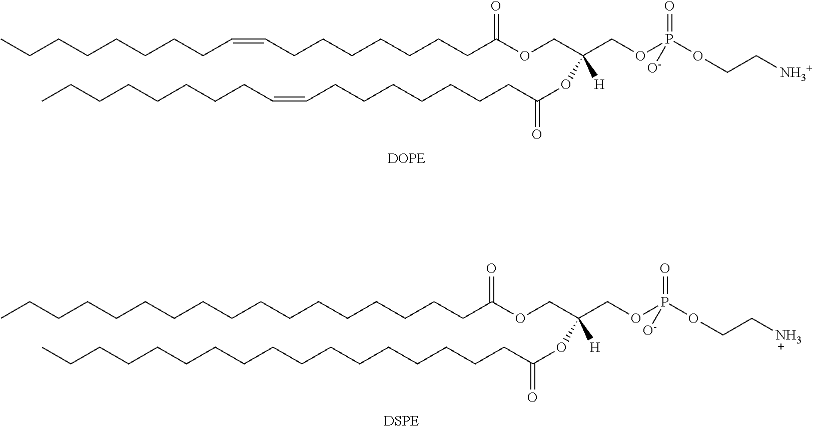 [SMILES: CCCCCCCC/C=C\CCCCCCCC(=O)OC[C@H](COP(=O)([O-])OCC[NH3+])OC(=O)CCCCCCC/C=C\CCCCCCCC ; CCCCCCCCCCCCCCCCCC(=O)OC[C@H](COP(=O)([O-])OCC[NH3+])OC(=O)CCCCCCCCCCCCCCCCC]

In some embodiments, the active ingredient will be encapsulated by the liposome so that the active ingredient is inaccessible to the aqueous medium. In other embodiments, the active ingredient will not be encapsulated by the liposome. In such embodiments, the active ingredient can be complexed on the outer surface of the liposome. In these embodiments, the active ingredient is accessible to the aqueous medium.

In certain preferred embodiments, the retinoid is 0.1 mol % to 20 mol % of the lipid molecules.

The forgoing compositions can also include PEG-conjugated lipids, which are known in the art per se, including PEG-phospholipids and PEG-ceramides, including one or more molecules selected from the following: PEG2000-DSPE, PEG2000-DPPE, PEG2000-DMPE, PEG2000-DOPE, PEG1000-DSPE, PEG1000-DPPE, PEG1000-DMPE, PEG1000-DOPE, PEG550-DSPE, PEG550-DPPE, PEG-550DMPE, PEG-1000DOPE, PEG-cholesterol, PEG2000-ceramide, PEG1000-ceramide, PEG750-ceramide, and PEG550-ceramide.

The foregoing compositions of the invention can include one or more phospholipids such as, for example, 1,2-distearoyl-sn-glycero-3-phosphocholine (“DSPC”), dipalmitoylphosphatidylcholine (“DPPC”), 1,2-dipalmitoyl-sn-glycero-3-phosphoethanolamine (“DPPE”), and 1,2-dioleoyl-sn-glycero-3-phosphoethanolamine (“DOPE”). Preferably, the helper phospholipid is DOPE.

The composition of the present invention may contain an active ingredient in the interior, may have an active ingredient attached to the exterior, or may be mixed with an active ingredient. Therefore, the composition of the present invention may be in the form of a complex between a liposome and an active ingredient, that is a lipoplex; depending on the administration route, the manner in which the drug is released, etc., the composition may be coated with an appropriate material such as for example an enteric coating or a timed disintegration material, or may be incorporated into an appropriate drug release system.

When a retinoid is contained as a targeting agent, the retinoid is present in a form in which it functions as a targeting agent in the present composition. Here, functioning as a targeting agent means that the composition containing a retinoid reaches and/or is incorporated into a collagen-producing cell, which is the target cell, in fibrotic tissue at a higher speed and/or in a larger amount than that of a composition not containing the retinoid, and this can be easily confirmed by for example adding a labeled or label-containing composition to a culture of target cells and analyzing the site where the label is present after a predetermined time has elapsed. In terms of the structure, for example, if a retinoid is at least partially exposed to the exterior of the composition at the latest before it reaches the target cell, the above-mentioned requirements can be satisfied. Whether or not a retinoid is exposed to the exterior of the composition may be evaluated by contacting the composition with a substance that specifically binds to a retinoid, for example, a retinol-binding protein (RBP), etc., and examining binding to the composition.

Exposing a retinoid at least partially to the exterior of the composition at the latest before it reaches a target cell may be carried out by for example adjusting the compounding ratio of the retinoid and the carrier-constituting component. Furthermore, when a lipid structure such as a liposome is utilized as a carrier, for example, when forming a complex between the lipid structure and the retinoid, a method in which the lipid structure is first diluted in an aqueous solution, and this is then contacted, mixed, etc., with the retinoid may be used. In this case, the retinoid may be in a state in which it is dissolved in a solvent, for example, an organic solvent such as DMSO. The lipid structure referred to here means any 3-dimensional structure, for example, a structure having a linear, film-like, spherical, etc. shape and containing a lipid as a constituent component, and examples thereof include, but are not limited to, a liposome, a micelle, a lipid microsphere, a lipid nanosphere, and a lipid emulsion. The possibility of application to another drug carrier of the same targeting agent as that used for targeting of a liposome is described in for example Zhao and Lee, Adv Drug Deliv Rev. 2004; 56(8): 1193-204, Temming et al., Drug Resist Updat. 2005; 8(6): 381-402, etc.

In addition to a collagen-reducing substance, the composition of the present invention may contain a substance that reduces a fibrotic stimulus as an active ingredient, or may be used in combination with such a substance. Examples of the substance that reduces a fibrotic stimulus include, but are not limited to, an antioxidant, a blood circulation promoter, an anti-inflammatory drug, an antiviral drug, an antibiotic, an antiparasitic agent, a liver protection drug, a choleretic drug, and an apoptosis suppressor. These substances may be selected as appropriate according to the tissue that is targeted and the disease state.

The composition of the present invention may contain a label. Labeling enables the success/failure of delivery to target cells, the increase/decrease of target cells, etc. to be monitored, and is useful not only at the test and research level but also at the clinical level. The label may be selected from any label known to a person skilled in the art such as for example any radioisotope, magnetic material, substance that binds to a labeled substance (e.g. an antibody), fluorescent substance, fluorophore, chemiluminescent substance, or enzyme. Labeling may be affixed to at least one constituent component of the composition of the present invention; for example, when a retinoid is contained as a targeting agent, it may be affixed to one or more of an active ingredient, the retinoid, and a carrier-constituting component, or labeling may be contained in the composition as a component other than the above.

The term ‘for collagen-producing cells in fibrotic tissue’ or ‘for delivery to collagen-producing cells in fibrotic tissue’ in the present invention means that it is suitable to use collagen-producing cells in fibrotic tissue as target cells, and this includes for example being able to deliver a substance to said cells at a higher speed, a higher efficiency, and/or in a larger amount than for other cells, for example, normal cells. For example, the carrier for collagen-producing cells in fibrotic tissue or the carrier for delivery to collagen-producing cells in fibrotic tissue can deliver an active ingredient to collagen-producing cells in fibrotic tissue at a speed and/or efficiency of at least 1.1 times, at least 1.2 times, at least 1.3 times, at least 1.5 times, at least 2 times and, moreover, at least 3 times compared with other cells. Since the composition of the present invention contains a targeting agent for collagen-producing cells in fibrotic tissue, it can be made as a composition for collagen-producing cells in fibrotic tissue or for delivery to collagen-producing cells in fibrotic tissue.

The composition of the present invention may be used as a medicine (that is, a pharmaceutical composition) and may be administered via various types of routes including oral and parenteral routes; examples thereof include, but are not limited to, oral, enteral, intravenous, intramuscular, subcutaneous, local, intrahepatic, intrabiliary, intrapulmonary, tracheobronchial, intratracheal, intrabronchial, nasal, intrarectal, intraarterial, intraportal, intraventricular, intramedullary, intra-lymph node, intralymphatic, intracerebral, intrathecal, intracerebroventricular, transmucosal, percutaneous, intranasal, intraperitoneal, and intrauterine routes, and it may be formulated in a dosage form that is suitable for each administration route. Such a dosage form and formulation method may be selected as appropriate from any known forms and methods (see e.g. ‘Hyojun Yakuzaigaku’ (Standard Pharmaceutical Science), Ed. by Yoshiteru Watanabe et al., Nankodo, 2003).

Examples of dosage forms suitable for oral administration include, but are not limited to, powder, granule, tablet, capsule, liquid, suspension, emulsion, gel, and syrup, and examples of dosage forms suitable for parenteral administration include injections such as an injectable solution, an injectable suspension, an injectable emulsion, and an injection in a form that is prepared at the time of use. Formulations for parenteral administration may be in a configuration such as an aqueous or nonaqueous isotonic aseptic solution or suspension.

The composition of the present invention may be supplied in any configuration, but from the viewpoint of storage stability, it is provided in a configuration that can be prepared at the time of use, for example in a configuration that allows a doctor and/or a pharmacist, a nurse, another paramedic, etc. to prepare it at the place of treatment or in the vicinity thereof. In this case, the composition of the present invention is provided as one or more containers containing at least one essential constituent element therefor, and it is prepared prior to use, for example, within 24 hours prior to use, preferably within 3 hours prior to use, and more preferably immediately prior to use. When carrying out the preparation, a reagent, a solvent, preparation equipment, etc. that are normally available in a place of preparation may be used as appropriate.

The present invention therefore also relates to a preparation kit for the composition, the kit including one or more containers containing singly or in combination an active ingredient and/or an optional targeting agent or carrier-constituting substance, and also relates to a constituent element necessary for the composition provided in the form of such a kit. The kit of the present invention may contain, in addition to the above, instructions, an electronic recording medium such as a CD or DVD, etc. related to a preparative method and administration method for the composition of the present invention, etc. Furthermore, the kit of the present invention may include all of the constituent elements for completing the composition of the present invention, but need not always include all of the constituent elements. Therefore, the kit of the present invention need not include a reagent or a solvent that is normally available at a place of medical treatment, an experimental facility, etc. such as, for example, sterile water, physiological saline, or a glucose solution.

In another aspect, the present disclosure relates to a pharmaceutical formulation comprising one or more physiologically acceptable surface active agents, pharmaceutical carriers, diluents, excipients, and suspension agents, or a combination thereof; and a formulation (e.g., the formulation that can include a compound, a retinoid, a second lipid, a stabilizing agent, and/or a therapeutic agent) disclosed herein. Acceptable additional pharmaceutical carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, and the like may be provided in the pharmaceutical formulation. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The pharmaceutical formulations described herein can be administered to a human patient per se, or in pharmaceutical formulations where they are mixed with other active ingredients, as in combination therapy, or suitable pharmaceutical carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may include, for example, parenteral delivery, including intramuscular, subcutaneous, intravenous, intramedullary injections, as well as intrathecal, direct intraventricular, intraperitoneal, intranasal, or intraocular injections. The formulation (e.g., the formulation that can include a compound, a retinoid, a second lipid, a stabilizing agent, and/or a therapeutic agent) can also be administered in sustained or controlled release dosage forms, including depot injections, osmotic pumps, and the like, for prolonged and/or timed, pulsed administration at a predetermined rate. Additionally, the route of administration may be local or systemic.

The pharmaceutical formulations may be manufactured in a manner that is itself known, e.g., by means of conventional mixing, dissolving, granulating, dragee-making, levigating, emulsifying, encapsulating, entrapping or tableting processes.

Pharmaceutical formulations may be formulated in any conventional manner using one or more physiologically acceptable pharmaceutical carriers comprising excipients and auxiliaries which facilitate processing of the active compounds into preparations which can be used pharmaceutically. Proper formulation is dependent upon the route of administration chosen. Any of the well-known techniques, pharmaceutical carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, sucrose, glucose, dextrose, mannitol, lactose, lecithin, albumin, sodium glutamate, cysteine hydrochloride, and the like. In addition, if desired, the injectable pharmaceutical formulations may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations may be utilized.

Pharmaceutical formulations for parenteral administration, e.g., by bolus injection or continuous infusion, include aqueous solutions of the active formulation (e.g., the formulation that can include a compound, a retinoid, a second lipid, a stabilizing agent, and/or a therapeutic agent) in water-soluble form. Additionally, suspensions of the active compounds may be prepared as appropriate oily injection suspensions. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the preparations described previously, the formulations may also be formulated as a depot preparation. Such long acting formulations may be administered by intramuscular injection. Thus, for example, the formulations (e.g., the formulation that can include a compound, a retinoid, a second lipid, a stabilizing agent, and/or a therapeutic agent) may be formulated with suitable polymeric or hydrophobic materials (for example as an emulsion in an acceptable oil) or ion exchange resins, or as sparingly soluble derivatives, for example, as a sparingly soluble salt.

Some embodiments herein are directed to a method of delivering a therapeutic agent to a cell. For example, some embodiments are directed to a method of delivering a therapeutic agent such as siRNA into a cell. Suitable cells for use according to the methods described herein include prokaryotes, yeast, or higher eukaryotic cells, including plant and animal cells (e.g., mammalian cells). In these embodiments, the formulations described herein can be used to transfect a cell. These embodiments may include contacting the cell with a formulation described herein that includes a therapeutic agent, to thereby deliver a therapeutic agent to the cell.

The present invention further relates to a method for regenerating normal tissue from fibrotic tissue, the method including a step of administering an effective amount of the composition or the collagen-reducing substance of the present invention to a subject that requires it. The effective amount referred to here is for example an amount that suppresses any increase in the amount of extracellular matrix such as collagen in fibrotic tissue, is preferably an amount that reduces the amount of extracellular matrix, and is more preferably an amount that causes regeneration of normal tissue in fibrotic tissue.

The amount of extracellular matrix may be quantitatively determined by various methods such as, for example, without limitation, image analysis of a specially stained image of extracellular matrix or measurement of an extracellular matrix marker. For example, collagen may be quantitatively determined by measuring the amount of a collagen marker such as hydroxyproline, or by subjecting tissue to collagen staining (e.g. Masson trichrome staining, Azan staining, sirius red staining, Elastica van Gieson staining, etc.) and carrying out an image analysis. The percentage reduction of extracellular matrix in fibrotic tissue may be for example at least 10%, at least 20%, at least 30%, at least 40%, at least 50%, at least 60%, at least 70% and, moreover, at least 75% compared with a case in which the composition of the present invention has not been administered. Here, the case in which the composition of the present invention has not been administered includes not only a case in which administration itself has not been carried out but also a case in which a vehicle alone has been administered, a case in which a composition corresponding to the composition of the present invention except that it does not contain the active ingredient has been administered and, when the composition of the present invention contains a targeting agent, a case in which a composition corresponding to the composition of the present invention except that it does not contain the targeting agent has been administered (so-called negative controls). Furthermore, regeneration of normal tissue may be evaluated by histological observation or by administration of labeled stem cells to fibrotic tissue and carrying out a tracking survey thereof.

The effective amount is preferably an amount that does not cause an adverse effect that exceeds the benefit from administration. Such an amount may be determined as appropriate by an in vitro test using cultured cells or by a test in a model animal such as a mouse, a rat, a dog, or a pig, and such test methods are well known to a person skilled in the art. Moreover, the dose of the drug used in the method of the present invention is known to a person skilled in the art, or may be determined as appropriate by the above-mentioned test, etc. As a model animal for fibrosis, various models such as a hepatic cirrhosis model obtained by carbon tetrachloride (CCl₄), porcine serum, dimethylnitrosamine (DMN), a methionine-choline deficient diet (MCDD), concanavalin A (Con A), bile duct ligation, etc., a pulmonary fibrosis model obtained by bleomycin (BLM), etc., a pancreatic fibrosis model obtained by dibutyltin dichloride, etc., and a myelofibrosis model such as a thrombopoietin (TPO) transgenic mouse (Leukemia Research 29: 761-769, 2005) may be used.

In the method of the present invention, the specific dose of the composition or collagen-reducing substance administered may be determined while taking into consideration various conditions with respect to the subject that requires the treatment, such as for example the severity of the symptoms, the general health condition of the subject, the age, weight, and gender of the subject, the diet, the timing and frequency of administration, a medicine used in combination, reaction to the treatment, compliance with the treatment, etc.

As the administration route, there are various routes including both oral and parenteral administration, and examples thereof include oral, enteral, intravenous, intramuscular, subcutaneous, local, intrahepatic, intrabiliary, intrapulmonary, tracheobronchial, intratracheal, intrabronchial, nasal, intrarectal, intraarterial, intraportal, intraventricular, intramedullary, intra-lymph node, intralymphatic, intracerebral, intrathecal, intracerebroventricular, transmucosal, percutaneous, intranasal, intraperitoneal, and intrauterine routes.

The frequency of administration depends on the properties of the composition used and the above-mentioned condition of the subject, and may be a plurality of times per day (that is, 2, 3, 4, 5, or more times per day), once a day, every few days (that is, every 2, 3, 4, 5, 6, or 7 days, etc.), a few times per week (e.g. 2, 3, 4 times, etc. per week), every week, or every few weeks (that is, every 2, 3, 4 weeks, etc.).

In the method of the present invention, the term ‘subject’ means any living individual, preferably an animal, more preferably a mammal, and yet more preferably a human individual. In the present invention, the subject may be healthy or affected by some disorder, but it typically means a subject having fibrotic tissue or tissue having a risk of becoming fibrotic. Examples of such a subject include, but are not limited to, a subject affected by the above organ fibrosis or having a risk of being affected and a subject for which tissue is receiving a fibrotic stimulus or has a risk of receiving it.

The present invention further relates to a method for regenerating normal tissue from fibrotic tissue, the method including a step of reducing collagen in the fibrotic tissue and/or a step of forming a space for cell growth and differentiation in the fibrotic tissue.

In the present method, reduction of collagen in fibrotic tissue and formation of a space for cell growth and differentiation may be carried out by administering the composition of the present invention or the above-mentioned collagen-reducing substance to fibrotic tissue.

The formulations or pharmaceutical compositions described herein may be administered to the subject by any suitable means. Non-limiting examples of methods of administration include, among others, (a) administration via injection, subcutaneously, intraperitoneally, intravenously, intramuscularly, intradermally, intraorbitally, intracapsularly, intraspinally, intrasternally, or the like, including infusion pump delivery; (b) administration locally such as by injection directly in the renal or cardiac area, e.g., by depot implantation; as well as as deemed appropriate by those of skill in the art for bringing the active compound into contact with living tissue.

Pharmaceutical compositions suitable for administration include formulations (e.g., the formulation that can include a compound, a retinoid, a second lipid, a stabilizing agent, and/or a therapeutic agent) where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of composition effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively dosages may be based and calculated upon the surface area of the patient, as understood by those of skill in the art.

The exact formulation, route of administration and dosage for the pharmaceutical compositions can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some condition, the dosages will be about the same, or dosages that are about 0.1% to about 500%, more preferably about 25% to about 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, a dose of about 0.1 mg to 2000 mg of each active ingredient, preferably about 1 mg to about 500 mg, e.g. 5 to 200 mg. In other embodiments, an intravenous, subcutaneous, or intramuscular dose of each active ingredient of about 0.01 mg to about 100 mg, preferably about 0.1 mg to about 60 mg, e.g. about 1 to about 40 mg is used. In cases of administration of a pharmaceutically acceptable salt, dosages may be calculated as the free base. In some embodiments, the formulation is administered 1 to 4 times per day. Alternatively the formulations may be administered by continuous intravenous infusion, preferably at a dose of each active ingredient up to about 1000 mg per day. As will be understood by those of skill in the art, in certain situations it may be necessary to administer the formulations disclosed herein in amounts that exceed, or even far exceed, the above-stated, preferred dosage range in order to effectively and aggressively treat particularly aggressive diseases or infections. In some embodiments, the formulations will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of formulation administered may be dependent on the subject being treated, on the subject's weight, the severity of the affliction, the manner of administration and the judgment of the prescribing physician.

Formulations disclosed herein (e.g., the formulation that can include a compound, a retinoid, a second lipid, a stabilizing agent, and/or a therapeutic agent) can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound formulated in a compatible pharmaceutical carrier may also be prepared, placed in an appropriate container, and labeled for treatment of an indicated condition.

It is understood that, in any compound described herein having one or more stereocenters, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound having one or more double bond(s) generating geometrical isomers that can be defined as E or Z each double bond may independently be E or Z a mixture thereof. Likewise, all tautomeric forms are also intended to be included.

### EXAMPLES

The present invention is explained in further detail by means of the Examples below, but they are only illustrations and do not in any way limit the present invention. In the Examples below, data are expressed as average values (±standard deviation). Multiple comparisons between a control group and another group were carried out by means of Dunnett's test.

### Example 1

### Preparation of VA-Lip siRNA

(1) Preparation of siRNA

As a sense strand and an antisense strand of siRNA (Hokkaido System Science Co., Ltd., Sapporo, Japan) targeted to the base sequence of gp46 (GenBank Accession No. M69246), which is the rat homologue of human HSP47, a molecular chaperone common to collagens (types I to IV), those below were used.

A:
(sense strand siRNA starting from the 757^{th} base
on the gp46 base sequence, SEQ ID NO: 5)
GUUCCACCAUAAGAUGGUAGACAACAG
B:
(antisense strand siRNA, SEQ ID NO: 6)
GUUGUCUACCAUCUUAUGGUGGAACAU

As siRNA random (also called siRNAscramble), those below were used.

C:
(sense strand siRNA, SEQ ID NO: 7)
CGAUUCGCUAGACCGGCUUCAUUGCAG
D:
(antisense strand siRNA, SEQ ID NO: 8)
GCAAUGAAGCCGGUCUAGCGAAUCGAU

In some experiments, sense strands having 6′-carboxyfluorescein (6-FAM) or fluorescein isothiocyanate (FITC) conjugated to the 5′ terminal were used. It was confirmed by a BLAST search that these sequences did not have homology with other known rat mRNA.

(2) Preparation of VA-Lip siRNA

As a cationic lipid, a cationic liposome (LipoTrust) containing O,O′-ditetradecanoyl-N-(α-trimethylammonioacetyl)diethanolamine chloride (DC-6-14), cholesterol, and dioleylphosphatidylethanolamine (DOPE) at a molar ratio of 4:3:3 was purchased from Hokkaido System Science Co., Ltd. (Sapporo, Japan). Before use, the liposome was prepared at a concentration of 1 mM (DC-6-14) by adding doubly distilled water (DDW) to a lyophilized lipid mixture while stirring. In order to prepare a VA coupled liposome, 200 nmol vitamin A (retinol, Sigma, USA) dissolved in DMSO was mixed with a liposome suspension (100 nmol as DC-6-14) in a 1.5 mL tube while stirring at 25° C. In order to prepare a VA coupled liposome supporting siRNAgp46 (VA-lip-siRNAgp46), an siRNAgp46 solution (580 pmol/mL in DDW) was added to the retinol coupled liposome solution while stirring at room temperature. The molar ratio of siRNA and DC-6-14 was 1:11. In order to obtain a desired dose in vitro, the VA-lip siRNA was reconstituted using phosphate buffered saline (PBS).

### Example 2

### Regenerative Therapy Experiment Using Hepatic Fibrosis Model Rat

### (1) Preparation of Hepatic Fibrosis Model Rat

A hepatic fibrosis model rat was prepared by subjecting a male SD rat (body weight 150 to 200 g) (Slc Japan, Shizuoka, Japan) to common bile duct ligation, and an individual on the 28th day after ligation was subjected to the present experiment. The present model rat was in a state in which cholestasis was caused by the common bile duct ligation and the liver tissue was continually exposed to a fibrotic stimulus.

### (2) Preparation of GFP-Labeled Rat Hepatic Stem Cells

GFP-labeled rat hepatic stem cells were harvested from the liver of a 4 week old GFP transgenic rat (Slc Japan). First, an EGTA solution and a collagenase solution were perfused through the GFP transgenic rat, the liver was then harvested, and the harvested liver was finely cut and then filtered using a cell strainer (pore diameter 100 μm). Hank's balanced salt solution (HBSS)+0.25% bovine serum albumin (BSA) solution were added to the cell suspension obtained, and the mixture was subjected to centrifugation at 4° C. and 500 rpm for 2 minutes. The supernatant was harvested and subjected to centrifugation at 4° C. and 1300 rpm for 5 minutes. After the supernatant was removed, MACS® (Magnetic Activating Cell Sorting) buffer (Miltenyi Biotec, Auburn, Calif., USA) was added to the precipitate and mixed. After the number of cells was counted, MACS® was carried out using an FITC conjugated mouse anti-CD45 antibody (BD Pharmingen), a rabbit polyclonal anti-CD133 antibody (Abcam), and a mouse monoclonal anti-EpCAM antibody (Santa Cruz), and CD133-positive, EpCAM-positive, and CD45 negative cells were harvested and used as rat hepatic stem cells in the present experiment.

### (3) Treatment of Hepatic Fibrosis Model Rat

The GFP-labeled hepatic stem cells prepared in (2) were locally transplanted in hepatic fibrosis model rats prepared in (1) at a concentration of 2×10⁶ counts in 200 μL of DME/F12 medium.

From 24 hours after transplantation of the hepatic stem cells, vitamin A coupled liposome-encapsulated siRNAgp46(VA-lip siRNAgp46) or VA-lip siRNAscramble as a mock was administered via the tail vein every other day a total of 12 times. The concentration of siRNA administered was 0.75 mg/kg rat body weight. The molar ratio of vitamin A, liposome (LipoTrust, Hokkaido System Science Co., Ltd., Sapporo, Japan), and siRNA was 11.5:11.5:1.

### (4) Tissue Staining

24 hours after the 12th administration of VA-lip siRNAgp46 in (3) (that is, on the 52nd day after the common bile duct ligation), the liver of the common bile duct ligation rat to which the GFP expressing hepatic stem cells had been transplanted was harvested. After the harvested liver was embedded using OCT compound, frozen sections were prepared. The liver sections were fixed using 4% paraformaldehyde. Some of the sections were subjected to Azan-staining by a standard method. Some of the sections were subjected to blocking with PBS containing 5% goat serum, washed with PBS, and then reacted at 4° C. overnight using a mouse monoclonal anti-a smooth muscle actin (α-SMA) antibody (Sigma), a mouse monoclonal anti-glial fibrillary acidic protein (GFAP) antibody (Sigma), a rabbit polyclonal anti-albumin antibody (MP Biomedicals), a mouse monoclonal anti-CK19 antibody (Novocastra), and a mouse monoclonal anti-vascular endothelium cadherin (ve-CAD, Vascular Endothelial Cadherin) antibody (Santa Cruz). After washing with PBS, they were reacted with an Alexa555-labeled goat anti-mouse IgG antibody and an Alexa555-labeled goat anti-rabbit IgG antibody (both from Invitrogen) at room temperature for 60 minutes. After washing with PBS, they were embedded using ProLong® Gold with DAPI (Invitrogen) and examined by means of a fluorescence microscope. Instead of the reaction with goat anti-rabbit antibody, some portion of the sections were reacted with an α-SMA antibody (Dako) and then subjected to coloration by means of diaminobenzidine (DAB) and further to nuclear staining by means of hematoxylin.

### Results

FIG. 1 shows the appearance of livers harvested from the test rats and Azan-stained images of representative sections thereof. In the group to which VA-lip siRNAscramble had been administered, the liver contracted, the surface was irregular, accumulation of extracellular matrix that had been stained blue was observed widely in the tissue in the Azan-stained image, and the hepatic lobule structure was disturbed. On the other hand, in the group to which VA-lip siRNAgp46 had been administered, there was no apparent contraction, the surface was smooth, there was hardly any accumulation of extracellular matrix in the tissue, and there was a clear reduction in the size of the fibrotic region compared with the VA-lip siRNAscramble-treated group. Furthermore, it was clearly observed that a normal hepatic lobule structure, in which the sinusoids run radially from the central vein, had recovered.

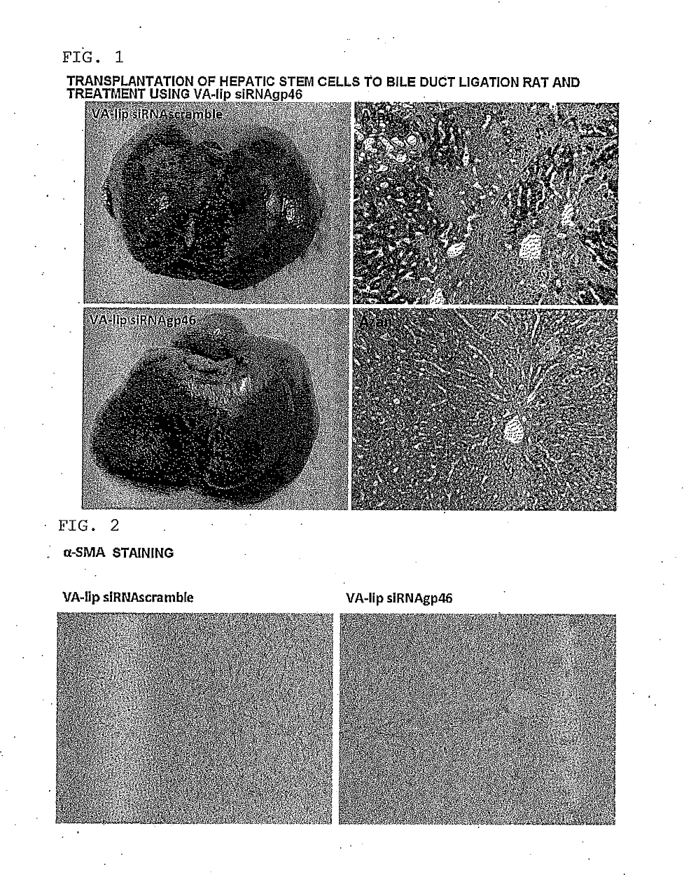

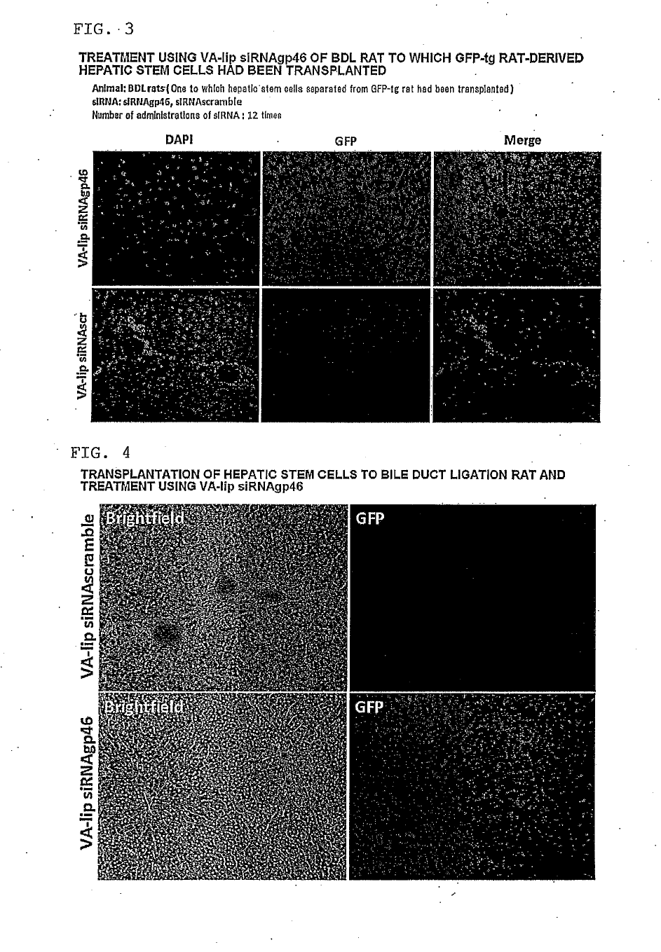

FIG. 2 shows α-SMA antibody DAB-stained images. Blue portions are hematoxylin-stained nucleus, and dark brown portions are α-SMA-positive regions. α-SMA is known as a marker for activated stellate cells, and it is thought that in the α-SMA-positive regions activated stellate cells are present. In the VA-lip siRNAgp46-treated group there was a marked reduction in the activated stellate cells compared with VA-lip siRNAscramble.

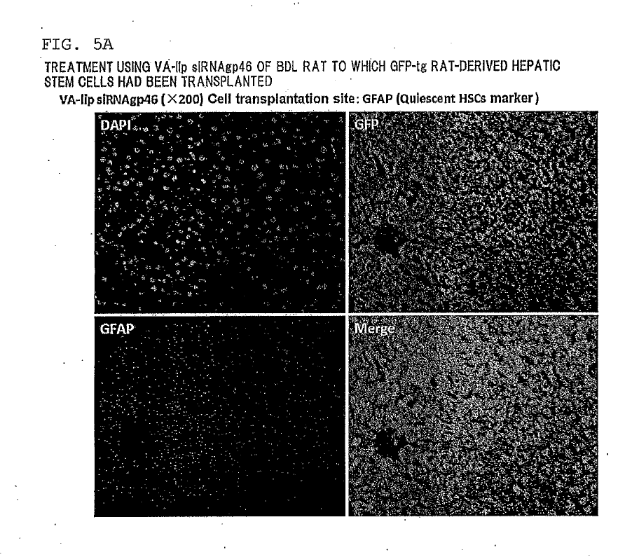

FIG. 3 shows DAPI and GFP fluorescence images of GFP-labeled hepatic stem cell transplantation sites. In the VA-lip siRNAgp46-treated group, GFP coloration was observed in about 80% of the region, whereas in the VA-lip siRNAscramble-treated group there was hardly any coloration.

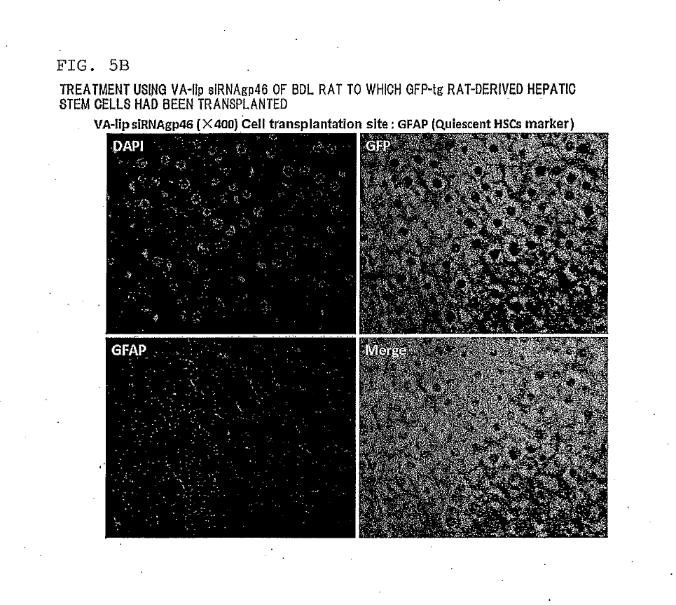

FIG. 4 shows bright field and GFP fluorescence images of GFP-labeled hepatic stem cell transplantation sites. In the VA-lip siRNAscramble-treated group, the shape of cells became blurred due to accumulation of extracellular matrix, particularly in areas around blood vessels, and the sinusoids ran in a random fashion, whereas in the VA-lip siRNAgp46-treated group the cell shape was clear and a sinusoid structure in which they ran radially from the central vein was observed. Furthermore, in the VA-lip siRNAscramble-treated group there was no GFP coloration, whereas in the VA-lip siRNAgp46-treated group GFP coloration was observed throughout the tissue.

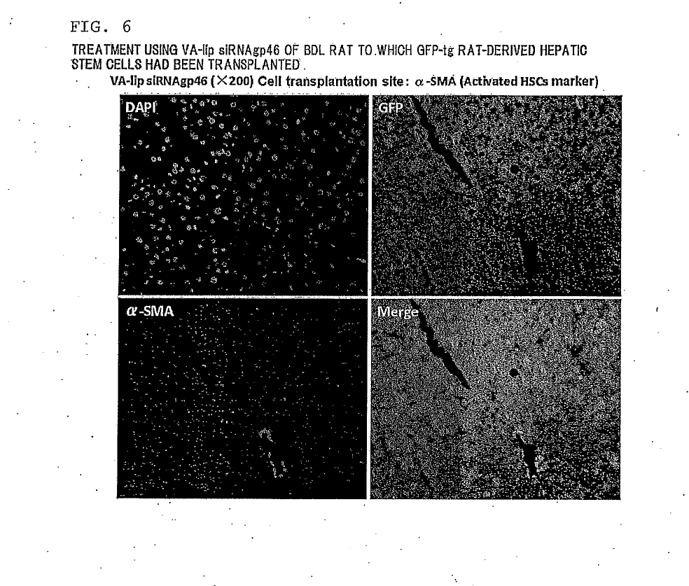

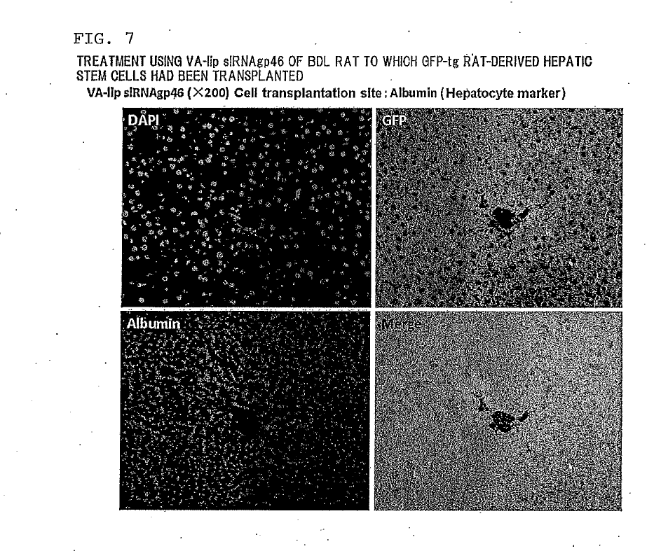

FIG. 5 is a comparison between DAPI and GFP fluorescence images and an image fluorescently stained by a GFAP antibody in the VA-lip siRNAgp46-treated group (FIG. 5A is 200× magnification and FIG. 5B is 400× magnification). GFAP is a protein known as a marker for hepatic stellate cells in a resting state. Cells expressing GFAP were not expressing GFP.

FIG. 6 is a comparison between DAPI and GFP fluorescence images and an image fluorescently stained by α-SMA antibody in the VA-lip siRNAgp46-treated group at 200× magnification. Cells expressing α-SMA were not expressing GFP. The results of FIGS. 5 and 6 suggest that hepatic stellate cells are not derived from hepatic stem cells.

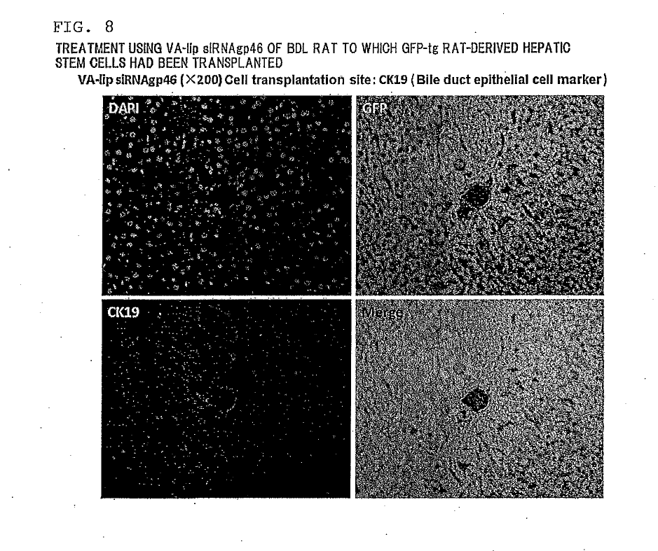

FIG. 7 is a comparison between DAPI and GFP fluorescence images and an image fluorescently stained by albumin antibody in the VA-lip siRNAgp46-treated group at 200× magnification. Albumin is a marker for hepatocytes, and many of the cells expressing GFP were expressing albumin.

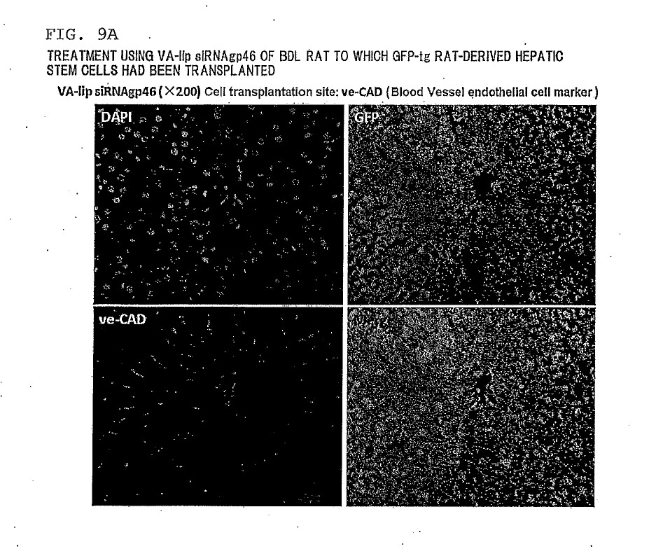

FIG. 8 is a comparison between DAPI and GFP fluorescence images and an image fluorescently stained by CK19 antibody in the VA-lip siRNAgp46-treated group at 200× magnification. CK19 is a marker for bile duct epithelial cells, and CK19-positive cells forming the bile duct were expressing GFP.

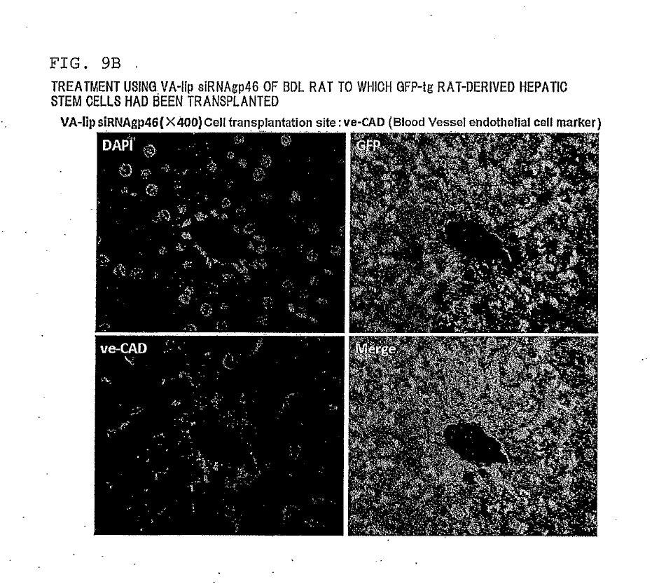

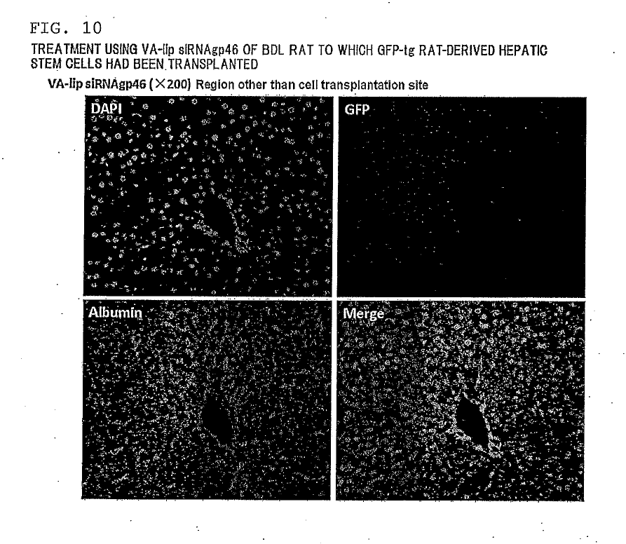

FIG. 9 is a comparison between DAPI and GFP fluorescence images and an image fluorescently stained by ve-CAD antibody in the VA-lip siRNAgp46-treated group (FIG. 9A is 200× magnification and FIG. 9B is 400× magnification). ve-CAD is known as a marker for blood vessel epithelial cells, and in some of the cells expressing GFP cells, cells expressing ve-CAD were observed.

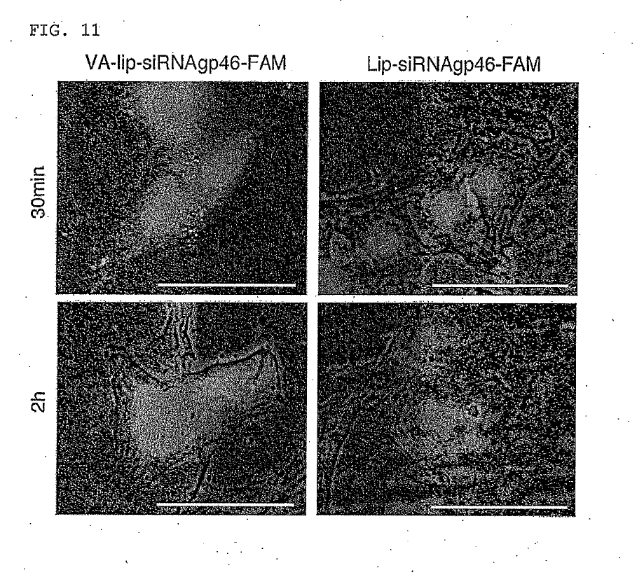

FIG. 10 is a comparison between DAPI and GFP fluorescence images and an image fluorescently stained by albumin antibody in a site of the VA-lip siRNAgp46-treated group where cells had not been transplanted at 200× magnification. In the site where cells had not been transplanted, there were no GFP-expressing cells.

### Discussion

Since cells that expressed GFP were cells derived from the transplanted hepatic stem cells, due to administration of VA-lip siRNAgp46, in the cell-transplantation site the fibrotic region reduced in size and hepatic stem cells differentiated to hepatocytes, bile duct epithelial cells, and blood vessel epithelial cells, thus showing that normal liver tissue was regenerated. That is, it has become clear that treatment involving administration of VA-lip siRNAgp46 not only cures hepatic fibrosis but also induces liver regeneration. Furthermore, the result that in the VA-lip siRNAscramble-treated group no hepatic stem cells could be detected (FIG. 3) suggests that the reduction in size of the fibrotic region due to VA-lip siRNAgp46 is deeply involved in the growth and differentiation of hepatic stem cells.

### Example 3

### Stellate Cell-Specific Delivery by Means of VA

### (1) Isolation of Rat Pancreatic Stellate Cells (PSC)

Rat pancreatic stellate cells (PSC) were isolated using a density gradient centrifugation method in accordance with a previous report (Apte et al. Gut 1998; 43: 128-133). Purity was assayed by microscopic examination, autofluorescence of endogenous VA, and an immunocytochemical method using a monoclonal antibody (1:25, Dako) for desmin, which is a muscle actin crosslinking protein. The viability of cells was assayed by trypan blue exclusion. Both the cell purity and the viability exceeded 95%. The cells were cultured in Iscove's modified Dulbecco's medium (Iscove's modified Dulbecco's medium: IMDM) supplemented with 10% fetal bovine serum (FBS) at 37° C. with 95% air/5% CO₂ under a humidified environment.

(2) Intracellular Distribution Analysis of VA-Lip siRNAgp46-FAM

Rat pPSCs (primary pancreatic stellate cells, primary PSC) were sown so that there were 1×10⁴ cells per chamber in a Lab-Tek chamber cover glass. VA-lip siRNAgp46-FAM or Lip siRNAgp46-FAM was added to the cells so that the final siRNA concentration was 50 nM. The cells were cultured in 10% FBS-containing DMEM for 30 minutes, and the medium was exchanged with fresh medium. 30 minutes after and 2 hours after the treatment the cells were washed with PBS three times, and were fixed by treating with 4% paraformaldehyde at 25° C. for 15 minutes. After fixation, the cells were washed with PBS three times and exposed to ProLong® Gold with DAPI (Invitrogen) for 1 minute to thus stain the nucleus. Intracellular localization of FAM-labeled siRNAgp46 was assayed using a fluorescence microscope (Keyence, BZ-8000).

(3) FACS Analysis of VA-Lip siRNAgp46-FAM

Rat pPSCs (1×10⁴ cells) were treated with VA-lip siRNAgp46-FAM (50 nM siRNA) in the presence of 10% FBS and cultured for 30 minutes. For a blocking assay, before VA-lip siRNAgp46-FAM was added, 1×10⁴ cells were treated with a mouse anti-RBP antibody (10 μg/mL, BD Pharmingen), or mouse IgG₁ (10 μg/mL, Dako) as a negative control, for 30 minutes. The mean fluorescence intensity (MFI) of VA-lip siRNAgp46-FAM-treated cells was assayed using a FACScalibur with CellQuest software (Becton Dickinson).

### (4) Western Blotting

In order to evaluate the knockdown effect of siRNAgp46, a Western blotting experiment was carried out. Specifically, protein extracts of PSCs respectively treated with VA-lip siRNAgp46 (1 nM, 5 nM, 50 nM), VA-lip-siRNA random (50 nM), and Lip-siRNAgp46 (50 nM) for 30 minutes were separated by means of 4/20 SDS-polyacrylamide gel, transferred to nitrocellulose film, probed with an antibody (Stressgen) for HSP47 (gp46) or an antibody (Cell Signaling) for β-actin, and labeled with a peroxidase-bound antibody (Oncogene Research Products, Boston, Mass.) as a secondary antibody. Finally, the cells were visualized by means of an ECL Western blotting detection system (Amersham Life Science, Arlington Heights, Ill.).

Furthermore, in order to confirm the duration of suppression of expression of gp46, PSCs were treated with VA-lip siRNAgp46 (50 nM) for 30 minutes and then cultured for 24 hours, 48 hours, 72 hours, and 96 hours, and following this protein was extracted and subjected to a Western blotting experiment in the same way as described above, together with one 30 minutes after treatment with VA-lip-siRNA random (50 nM).

### (5) Quantitative Determination of Production of Collagen

Rat pPSCs were sown on a 6-well tissue culture plate at a density of 5×10⁴ cells/well in 10% FBS-containing DMEM. After culturing for 24 hours, the rat pPSCs were treated with VA-lip siRNAgp46 (50 nM siRNA) and VA-lip siRNA random (50 nM siRNA). The cells were cultured in 10% FBS-containing DMEM for 30 minutes, and the medium was then exchanged with fresh medium. 72 hours after the treatment, the cells were washed with PBS three times, and collagen deposited in the well was stained using sirius red (Biocolor, Belfast, UK) in accordance with a previous report (Williams et al. Gut 2001; 49: 577-583). Unbound dye was removed by washing, and bound complex was dissolved in 0.5% sodium hydroxide. Quantitative analysis of collagen was carried out by absorption intensity analysis at 540 nm, and the result was expressed as a percentage relative to an untreated control. Results

FIG. 11 shows fluorescence images of the intracellular distribution of FAM-labeled siRNA. The two images on the left are fluorescence images of PSCs treated with VA-lip siRNAgp46-FAM, and the two images on the right are fluorescence images of PSCs treated with Lip siRNAgp46-FAM. The upper two images are images 30 minutes after the treatment, and the lower two images are images 2 hours after the treatment. 30 minutes after the treatment With VA-lip siRNAgp46-FAM, faint green fluorescence due to FAM in a granular pattern was observed within the cytoplasm, and 2 hours after the treatment, a darker granular pattern was observed in a region around the nucleus. In comparison therewith, in the Lip siRNAgp46-FAM-treated group, no green fluorescence was observed 30 minutes after the treatment, and fluorescence around the nucleus 2 hours after the treatment was faint.

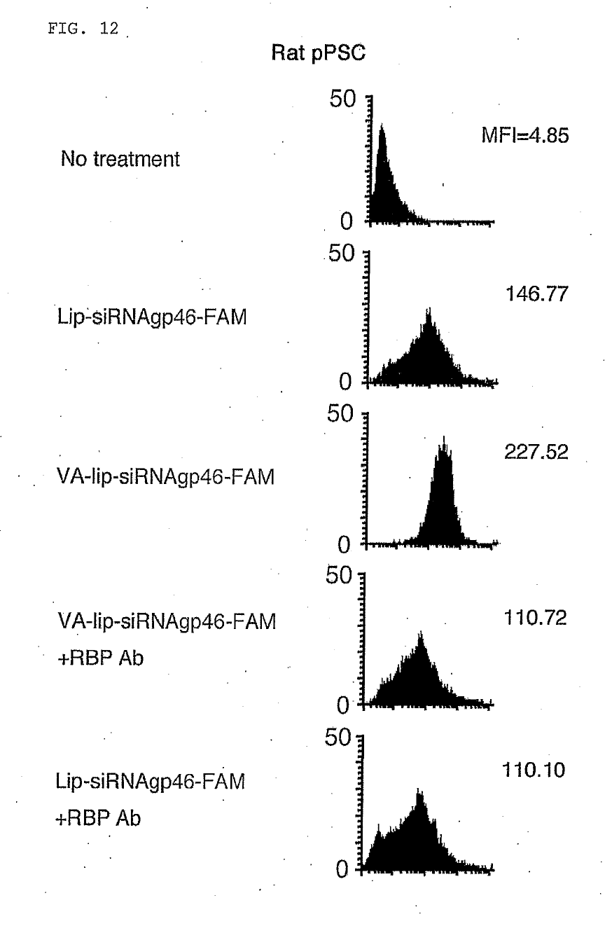

FIG. 12 shows graphs of the results of the FACS analysis. The results of the non-treated group, the Lip siRNAgp46-FAM-treated group, the VA-lip siRNAgp46-FAM-treated group, the VA-lip siRNAgp46-FAM+RBP antibody-treated group, and the Lip siRNAgp46-FAM+RBP antibody-treated group are shown in sequence from the top. In the results of the FACS analysis, compared with the VA-lip siRNAgp46-FAM-treated group, in the VA-lip siRNAgp46-FAM+RBP antibody-treated group, the fluorescence strength was suppressed to the same level as that of the Lip siRNAgp46-FAM-treated group, suggesting that the incorporation of VA-lip siRNAgp46 into PSCs is mediated by an RBP receptor.

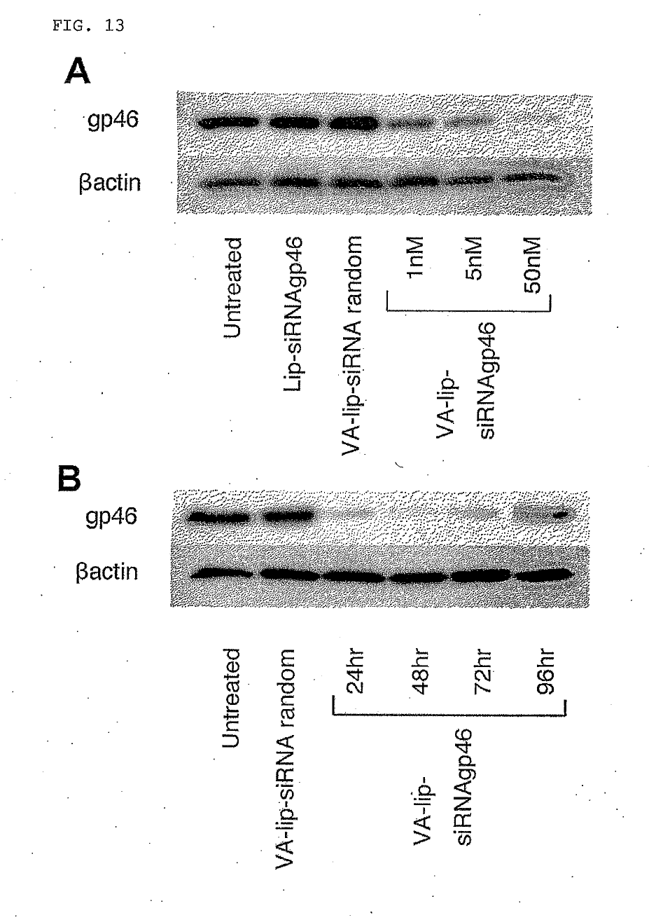

FIG. 13A shows the results of Western blotting, which show the difference in suppression effect according to concentration. In the cells treated with VA-lip siRNAgp46, suppression of the expression of gp46 was observed to be dependent on the concentration of VA-lip siRNAgp46, the expression being almost completely suppressed at 50 nM, whereas suppression of expression was not observed with VA-lip siRNA random or Lip siRNAgp46.

FIG. 13B shows the result of Western blotting for ascertaining the duration of the suppression effect. When treated with VA-lip siRNAgp46, in cells cultured for 72 hours after the treatment, marked suppression of gp46 was observed. Therefore, it was confirmed that the effect of suppressing the expression of gp46 continued for at least 72 hours after the treatment.

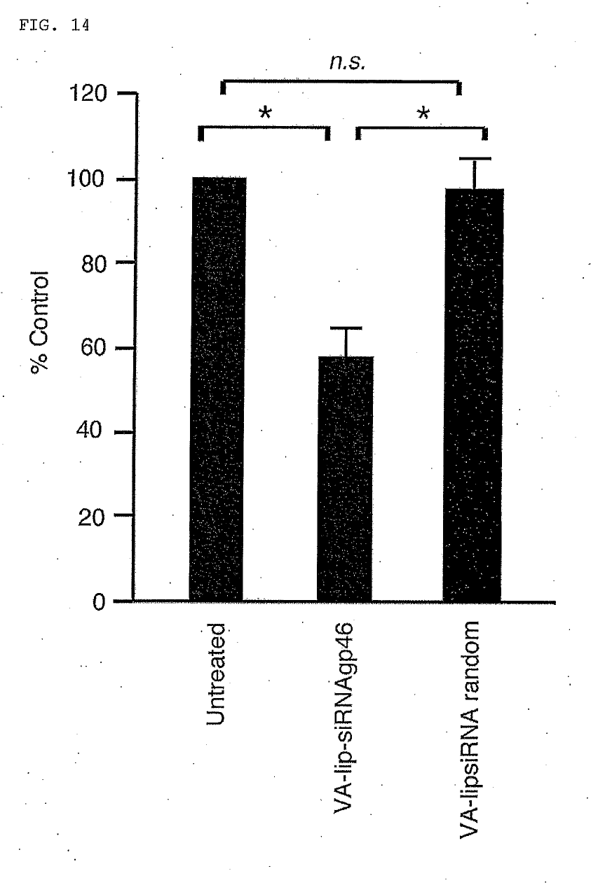

FIG. 14 is a graph showing quantitative determination of the amount of collagen produced after 72 hours in non-treated cells and cells treated with VA-lip siRNAgp46 and VA-lip siRNA random respectively. Compared with the untreated cells and the cells treated with VA-lip siRNA random, when treated with VA-lip siRNAgp46, marked suppression of the production of collagen was confirmed.

### Discussion

From the results above it can be seen that, in vitro, VA-lip siRNAgp46 is incorporated specifically into PSCs by RBP receptor-mediated incorporation to thus suppress the expression of gp46, and as a result, the production of collagen is markedly suppressed. This suggests that in pancreas affected by pancreatic fibrosis, VA-lip siRNAgp46 can reduce collagen.

### Example 4

### Experiment of Regenerative Therapy of Pancreatic Fibrosis Model Rat

### (1) Preparation of Pancreatic Fibrosis Model Rat

Male Lewis rats having a body weight of 150 to 200 g (Charles River) were used. In accordance with a previous report (Inoue et al. Pancreas 2002; 25: e64-70), dibutyltin dichloride (Dibutyltin dichloride, DBTC) was dissolved in 1 part of ethanol and then mixed with 2 parts of glycerol and 2 parts of dimethyl sulfoxide (DMSO) to thus prepare a solution (DBTC solution), and an amount corresponding to 5 mg (DBTC)/kg (body weight) was administered to the rat right carotid artery by means of a syringe.

(2) In Vivo Localization of VA-Lip siRNAgp46-FITC in Rat Pancreas and Other Tissue

After 43 days from starting administration of DBTC, at the point when serious pancreatic fibrosis was observed, 1 μL/g body weight of VA-lip siRNAgp46-FITC or Lip siRNAgp46-FITC was administered to the DBTC-treated rat via the tail vein. Administration was carried out under normal pressure three times every other day with 0.75 mg/kg of siRNA each time. 24 hours after the final administration, the rat was sacrificed by perfusion with physiological saline, and the pancreas and other organs (the liver, the lung, the spleen, and the retina) were harvested. The organ samples were fixed with 10% paraformaldehyde, and paraffin-embedded sections were stained using Azan-Mallory stain. Immunohistochemical staining was carried out by the dextran polymer method using each of a monoclonal anti-α-SMA antibody (1:1000, Sigma), an anti-CD68 antibody (1:500, Dako), and an anti-FITC antibody (1:500, Abcam) and by means of an Envision Kit (Dako), and following coloration by means of DAB (Wako Pure Chemical Industries, Ltd., Osaka, Japan) and nuclear staining by means of Gill's hematoxylin solution (Wako Pure Chemical Industries, Ltd.) were carried out.

### (3) Western Blotting

In order to evaluate the duration of suppression of expression by means of siRNAgp46 in vivo, protein extracts from the pancreas 0, 1, 2, 3, and 4 days after intravenous administration of VA-lip siRNAgp46 were subjected to Western blotting in the same way as for Example 3.(4).

(4) In Vivo siRNAgp46 Treatment

Three groups of rats (n=6 per group) were used for histological evaluation. 43 days after administration of DBTC, each group was treated with administration of PBS, VA-lip siRNA random, and VA-lip siRNAgp46 10 times respectively (0.75 mg/kg siRNA, administered three times every other day). All administrations were carried out via the tail vein under normal pressure with an amount of 1 μL/g body weight. The pancreas was fixed with 10% paraformaldehyde and embedded in paraffin, and a section was then strained using Azan-Mallory stain and hematoxylin-eosin stain Immunohistochemical staining was carried out by the dextran polymer method using a monoclonal anti-α-SMA antibody (1:1000, Sigma) and by means of an Envision Kit (Dako), and subsequently coloration by means of DAB (Wako Pure Chemical Industries, Ltd., Osaka, Japan) and nuclear staining by means of Gill's hematoxylin solution (Wako Pure Chemical Industries, Ltd.) were carried out. In order to carry out precise quantitative determination of regions stained by means of Azan-Mallory, hematoxylin-eosin, and α-SMA, six low magnification fields (100×) were randomly selected for each rat pancreatic section and examined using a microscope (Axioplan 2; Carl Zeiss, Inc). A digital image was taken by means of a video recording system using a digital TV camera system (Axiocam High Resolution color, Carl Zeiss, Inc.). The proportion of the region stained by Azan-Mallory and α-SMA in a digital microscope photograph was determined using an automatic software analysis program (KS400, Carl Zeiss, Inc.).

### (5) Hydroxyproline Assay

Hydroxyproline content was determined by the Weidenbach method in accordance with a previous report (Weidenbach et al. Digestion 1997; 58: 50-57). In brief, pancreatic cell debris was centrifuged at 3000 rpm for 15 minutes, a pellet was completely hydrolyzed in 6 N HCl at 96° C. for 16 hours, the pH was adjusted to 6.5 to 7.5, and it was subjected again to centrifugation (at 3000 rpm for 15 minutes). 25 μL of an aliquot was dried at 60° C., and the precipitate was dissolved in 1.2 mL of 50% isopropanol and incubated in 200 mL of acetic acid/citric acid buffer (pH 6.0) containing 0.56% chloramine T Solution (Sigma). After incubating at 25° C. for 10 minutes, 1 mL of Ehrlich's reagent was added, and the mixture was incubated at 50° C. for 90 minutes. After cooling, the absorption at a wavelength of 560 nm was measured.

### (6) Collagenase Activity of Pancreatic Cell Debris

Measurement of collagenase activity was carried out by a modified method of a previous report (Iredale et al. J. Clin. Invest. 1998; 102: 538-549). In brief, pancreas harvested from a wild-type rat and a pancreatic fibrosis model rat and frozen with liquid nitrogen were crushed on ice in a sample buffer (50 mM Tris, pH 7.6, 0.25% Triton X-100, 0.15 M NaCl, 10 mM CaCl₂) containing a serine and thiol protease inhibitor (PMSF 0.1 mM, leupeptin 10 μM, pepstatin A 10 μM, aprotinin 25 μg/mL, iodoacetamide 0.1 mM). The cell debris was centrifuged at 4° C. and 14000 g for 30 minutes, thus removing cell residue and protein aggregate. The collagenase activity in the pancreatic cell debris was determined using an EnzCheck Collagenase Assay Collagen Conjugate kit (Molecular Probes) in accordance with the instruction manual. In parallel thereto, analysis was carried out using an appropriate negative control and positive control (bacterial collagenase), and the results were expressed as fluorescence of degraded collagen per mg of protein (determined by optical density at 280 nm compared with serum albumin standard).

### Results

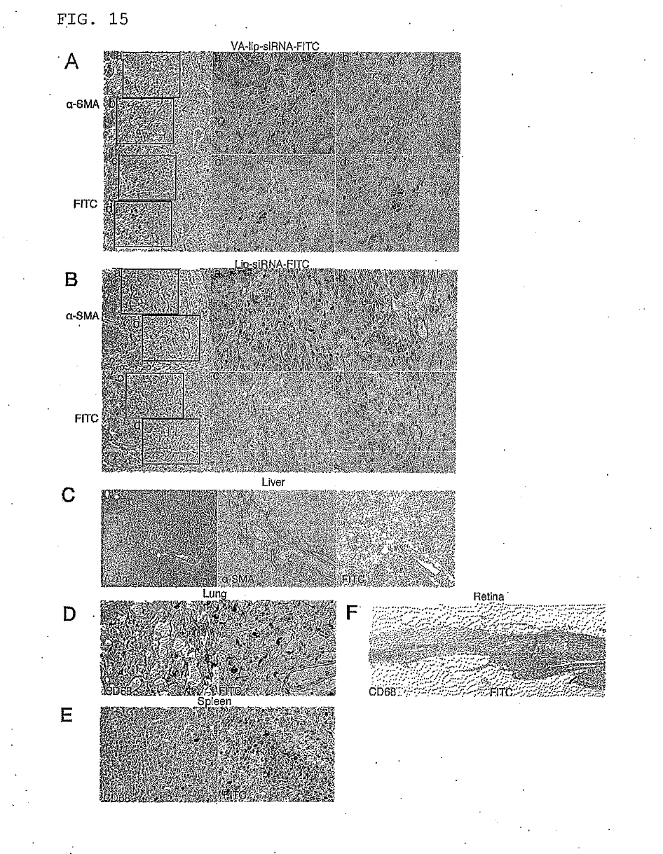

In consecutive sections of the pancreas, activated stellate cells and siRNAgp46-FITC were immunostained, and the results were that in the VA-lip siRNAgp46-FITC-treated group, in a region where activated stellate cells (α-SMA-positive cells) aggregated, FITC-positive cells were identified, whereas in the Lip siRNAgp46-FITC-treated group, the number of FITC-positive cells identified in an α-SMA-positive region was very small (FIGS. 15A and B).

FITC-positive cells in an α-SMA-positive region were also observed in a liver sample (FIG. 15C). This result coincides with the knowledge that DBTC not only induces pancreatic fibrosis but also hepatic cirrhosis. In other rat organs, including the lung and the spleen, few cells were stained with FITC in a region with macrophage infiltration (CD68-positive cells) (FIGS. 15D and E), suggesting nonspecific incorporation of siRNAgp46-FITC by macrophages. The retina was negative in FITC staining (FIG. 15F), and this coincides with the knowledge obtained using VA-lip siRNAgp46-FAM in hepatic cirrhosis. It is thought that the eyeball probably constructs an independent system due to the low permeability of the blood-retina barrier.

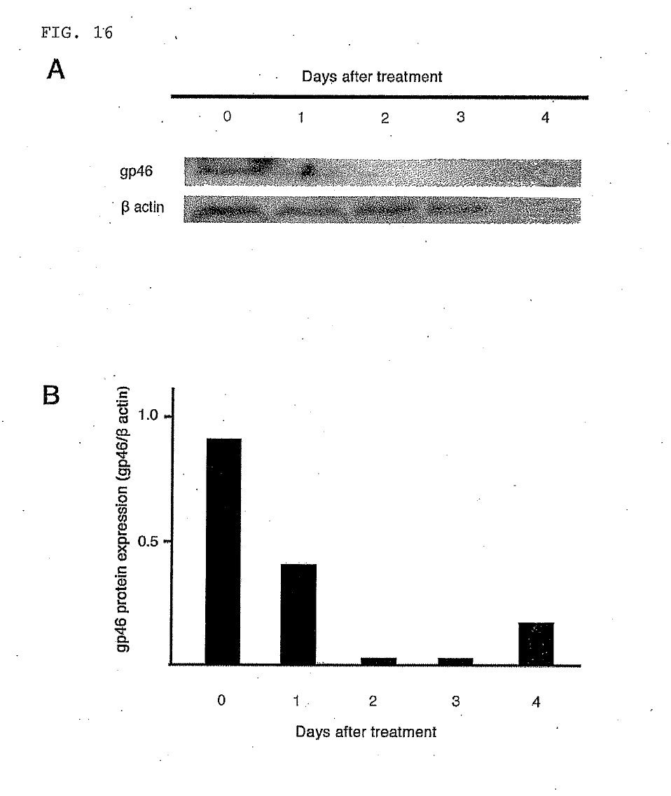

It was confirmed from the results of Western blotting that, in vivo also, the effect of siRNAgp46 in suppressing the expression of gp46 continued for at least 3 days (FIGS. 16A and B).

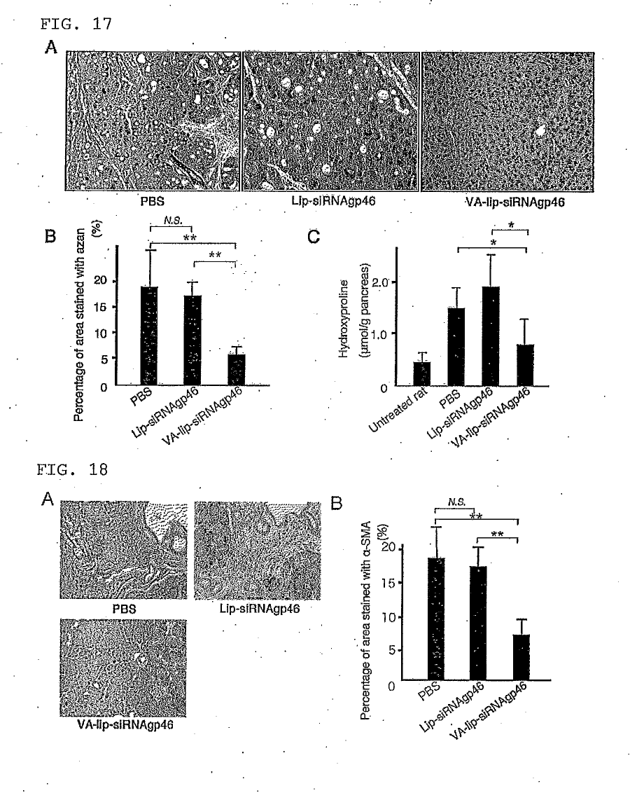

A DBTC-treated rat to which VA-lip siRNAgp46 had been administered 10 times was evaluated by Azan-Mallory staining (FIG. 17A). The fibrotic region as determined by computer image analysis was markedly reduced in a sample from the VA-lip siRNAgp46-treated group compared with a control sample (P<0.01) (FIG. 17B). This result coincided with data showing clear suppression of hydroxyproline in the pancreas of the VA-lip siRNAgp46-treated group (FIG. 17C).

In order to evaluate change in stellate cells in the rat pancreas after treatment with VA-lip siRNAgp46, a rat pancreas sample after treatment with VA-lip siRNAgp46 was subjected to α-SMA staining, and the result showed that the number of α-SMA-positive cells markedly decreased compared with that of a rat treated with Lip siRNAgp46 and PBS (FIGS. 18A and B).

The collagenase activity in pancreatic cell debris of a wild-type rat and a VA-lip siRNAgp46-treated DBTC-treated rat was measured based on the assumption that improvement of fibrosis subsequent to suppression of the secretion of new collagen from PSCs by administration of VA-lip siRNAgp46 involves collagenase derived from inflammatory cells and PSCs themselves, and the results are shown in the table below.

TABLE 2
Collagenase activity in rat pancreatic cell debris

| | Collagenase activity |
|---|---|
| | (arbitrary units of fluorescence/mg protein) |
| Normal rat | 20500 ± 300 |
| DBTC rat (29th day) | 26300 ± 700 |
| DBTC rat (57th day) | 25400 ± 1000 |

Numerical values are average values ± standard deviation (n = 5 for each group)

As shown in the table, the collagenase activity in the DBTC-treated rat was almost the same as that of the wild-type rat.

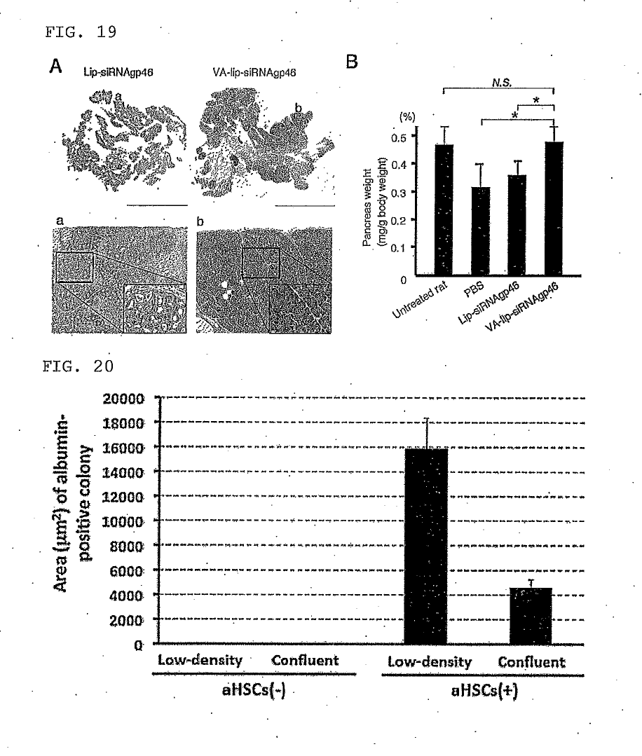

When comparing the hematoxylin-eosin staining images of the pancreatic samples of the VA-lip siRNAgp46-treated and Lip siRNAgp46-treated DBTC-treated rats on the 65th day, in the VA-lip siRNAgp46-treated rat, although not complete, a clear normalization of pancreatic tissue was observed, whereas in the Lip siRNAgp46-treated rat tissue normalization was not observed (FIG. 19A). This coincided with normalization of the pancreatic weight of the VA-lip siRNAgp46-treated DBTC-treated rat (FIG. 19B).

### Discussion

From the above-mentioned results, it can be seen that due to treatment with VA-lip siRNAgp46, siRNAgp46 is specifically incorporated into activated pancreatic stellate cells (aPSCs) to thus suppress the expression of gp46; as a result, secretion of collagen from aPSCs is suppressed, and a marked effect in the improvement of pancreatic fibrosis is thereby exhibited. Furthermore, a marked decrease in aPSCs was observed, which is probably due to a reduction in the secretion of collagen. It is worthy of special note that treatment with VA-lip siRNAgp46 not only improves pancreatic fibrosis but also induces regeneration of pancreatic tissue. Taking this into consideration together with the results of Example 2 above, these results suggest that reducing collagen accumulated in fibrotic tissue enables normal tissue to be tissue-nonspecifically regenerated from fibrotic tissue.

### Example 5

### Importance of Space for Growth and Differentiation of Stem Cells

Activated hepatic stellate cells (aHSCs) were cocultured with various densities of hepatic progenitor cells, and the effect of the existence of space around the cells on the differentiation of hepatic progenitor cells was examined. As hepatic progenitor cells, GFP-labeled rat hepatic stem cells obtained in Example 2(2) above were used, and as the aHSCs, HSCs harvested from an SD rat, cultured, and passaged once were used. The aHSCs were harvested and cultured as follows. First, an SD rat was perfused with EGTA solution and a collagenase solution, the liver was harvested, and the harvested liver was finely cut and filtered using a cell strainer (pore diameter 100 μm). An HBSS+0.25% BSA solution was added to the cell suspension thus obtained, and the mixture was centrifuged at 4° C. and 500 rpm for 2 minutes. The supernatant was harvested and centrifuged at 4° C. and 1300 rpm for 5 minutes. After the supernatant was removed, an HBSS+0.25% BSA solution was added, and a 28.7% Nycodenz solution (Axis Shield, Oslo, Norway) was added so that the concentration of Nycodenz was 13.2%, and mixed. After layering an HBSS+0.25% BSA solution, centrifugation was carried out at 4° C. and 1400×g for 20 minutes. After the centrifugation was complete, an intermediate layer was harvested and cultured using Dulbecco's Modified Eagle's medium (DMEM)+10% fetal bovine serum (FBS) medium for 5 days. Passaging was carried out on the fifth day of culturing, and the cells were used in the present experiment.

aHSCs were sown on cell culture inserts (pore diameter 0.4 μm, BD Falcon, Franklin Lakes, N.J., USA) at a density of 5×10⁴ cells/well and cultured in an incubator at 37° C. and 5% CO₂ using DMEM+10% FBS for 48 hours. 2 days after sowing the aHSCs, hepatic progenitor cells were sown on a 24-well plate (BD Falcon) equipped with a type I collagen-coated cover glass (IWAKI, Tokyo, Japan) at a density of 1×10⁴ cells/well (low density) and 5×10⁵ cells/well (confluent). Subsequently, the above-mentioned cell culture inserts containing aHSCs were inserted into the wells of the 24-well plate and cocultured in an incubator at 37° C. and 5% CO₂ for 10 days (as medium, DME/F12 (Dulbecco's Modified Eagle's Medium/Nutrient F-12 Ham)+10% FBS+ITS (10 mg/L insulin, 5.5 mg/L transferrin, 0.67 μg/L selenium)+0.1 μM dexamethasone+10 mM nicotinamide+50 μg/mL β-mercaptoethanol+2 mM L-glutamine+5 mM Hepes was used).

On the 10th day of coculturing, immunostaining was carried out using an anti-albumin antibody (rabbit polyclonal, MP Biomedicals), albumin-positive colonies were imaged using an inverted microscope (Nikon) at a magnification of 100×, and based on the image obtained the area of albumin-positive colonies was calculated using NIS-Elements software (Nikon). The results are shown in FIG. 20.

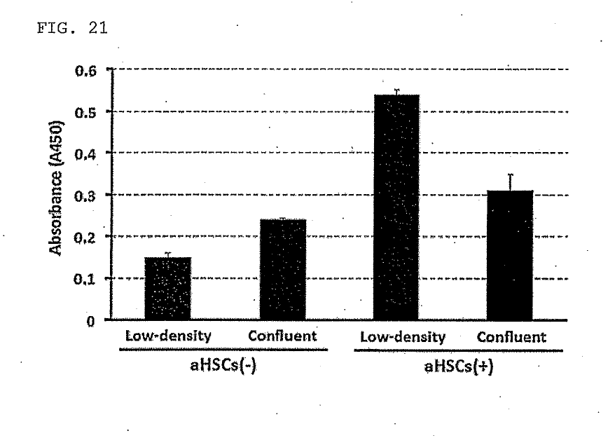

In a different experiment, on the 10th day of coculturing, measurement of cell growth was carried out using a Premix WST-1 Cell Proliferation Assay System (Takara, Tokyo, Japan) with a microplate reader (Bio-Rad Laboratories, Hercules, Calif., USA). The results are shown in FIG. 21.

From the results shown in FIG. 20, it was clear that, when aHSCs were cocultured with hepatic progenitor cells sown at a low density, the hepatic progenitor cells differentiated into a large number of albumin-positive hepatocytes, but when the hepatic progenitor cells were confluent, only a very small number differentiated into hepatocytes. When hepatic progenitor cells were monocultured, they did not differentiate into albumin-positive hepatocytes. Furthermore, as shown in FIG. 21, when the hepatic progenitor cells were sown at the same density as above, the proliferation potency thereof was smaller under confluent conditions than at low density conditions.

From the above results, it has been found that activated stellate cells induce growth and differentiation of stem cells, and the existence of a physical space around stem cells has an important effect on the growth and differentiation of stem cells. When this is taken into consideration together with the results of the Examples above, it shows that a collagen-reducing substance causes a reduction of fibrous tissue in fibrotic tissue, space is formed around stem cells, and as a result the stem cells grow and differentiate, thus regenerating normal tissue.

### Example 6

### Synthesis of DOPE-Glu-VA

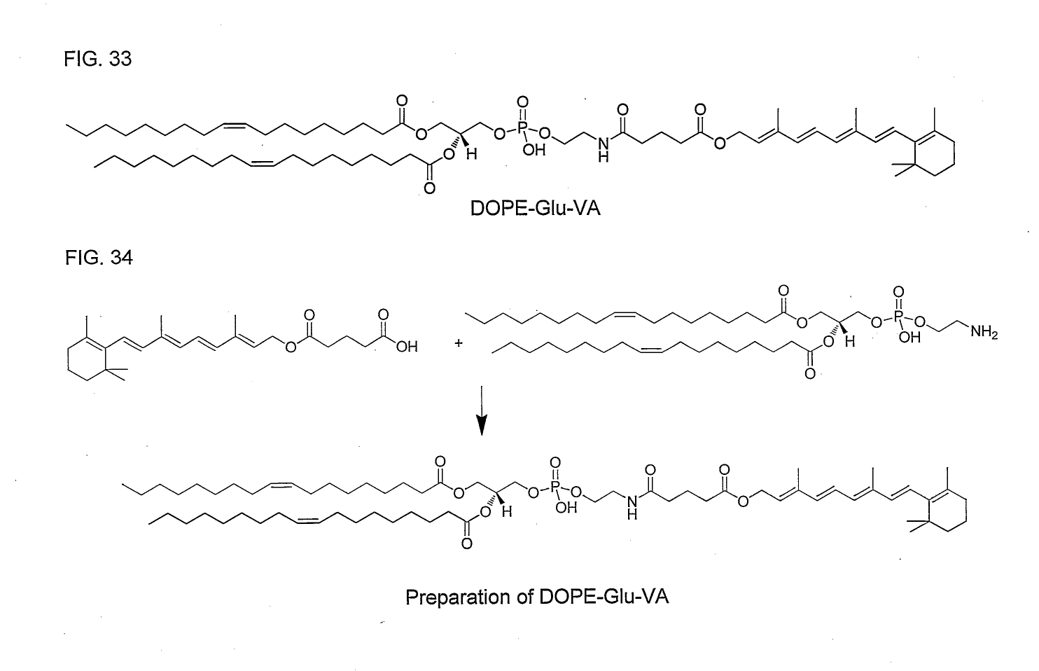

### Preparation of (Z)-(2R)-3-(((2-(5-(((2E,4E,6E,8E)-3,7-dimethyl-9-(2,6,6-trimethylcyclohex-1-en-1-yl)nona-2,4,6,8-tetraen-1-yl)oxy)-5-oxopentanamido)ethoxy)(hydroxy)phosphoryl)oxy)propane-1,2-diyl dioleate (DOPE-Glu-VA) (see FIG. 33)

### Preparation of Intermediate 1: 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid

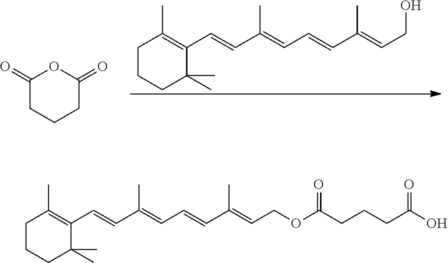

Glutaric anhydride (220 mg, 1.93 mmol) and retinol (500 mg, 1.75 mmol) were dissolved in dichloromethane (5 mL) in an amber-colored vial. Triethylamine (513 ul, 3.68 mmol) was added and the vial was flushed with argon. Reaction mixture was allowed to stir at room temperature for 4 hours. The material was concentrated and purified by silica gel chromatography with a dichloromethane/methanol gradient. Fractions were pooled and concentrated to yield yellowish oil (700 mg). The product was verified by NMR.

### Preparation of DOPE-Glu-VA: (Z)-(2R)-3-(((2-(5-(((2E,4E,6E,8E)-3,7-dimethyl-9-(2,6,6-trimethylcyclohex-1-en-1-yl)nona-2,4,6,8-tetraen-1-yl)oxy)-5-oxopentanamido)ethoxy)(hydroxy)phosphoryl)oxy)propane-1,2-diyl dioleate (see FIG. 34)

1,2-dioleoyl-sn-glycero-3-phosphoethanolamine (500 mg, 0.672 mmol), N,N,N′,N′-Tetramethyl-O-(7-azabenzotriazol-1-yl)uronium hexafluorophosphate (306.5 mg, 0.806 mmol) and 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid (269 mg, 0.672 mmol) was dissolved in chloroform/DMF (10 mL, 1:1 mixture) in an amber-colored vial flushed with argon and N,N-Diisopropylethylamine (300 μL, 1.68 mmol) was added. Reaction mixture was allowed to stir overnight at room temperature. The reaction mixture was concentrated and then purified by silica gel chromatography using a dichloromethane/methanol gradient. The fractions were pooled and concentrated to yield yellowish oil (460 mg, 61%). Verified product by NMR. ¹H NMR (400 MHz), δ_{H}: 8.6 (d, 1H), 8.27 (d, 1H), 6.57-6.61 (dd, 1H), 6.08-6.25 (m, 4H), 5.57 (t, 1H), 5.30-5.34 (m, 4H), 5.18 (m, 1H), 4.68-4.70 (d, 2H), 4.28-4.35 (m, 1H), 4.05-4.15 (m, 1H), 3.81-3.97 (m, 4H), 3.52-3.62 (m, 1H), 3.35-3.45 (m, 2H), 2.95-3.05 (m, 1H), 2.33-2.35 (t, 3H), 2.2-2.3 (m, 7H), 1.9-2.05 (m, 17H), 1.85 (s, 3H), 1.69 (s, 3H), 1.5-1.65 (m, 6H), 1.4-1.5 (m, 2H), 1.18-1.38 (m, ˜40H), 1.01 (s, 3H), 0.84-0.88 (m, 12H).

### Example 7

### DOPE-Glu-NH-VA

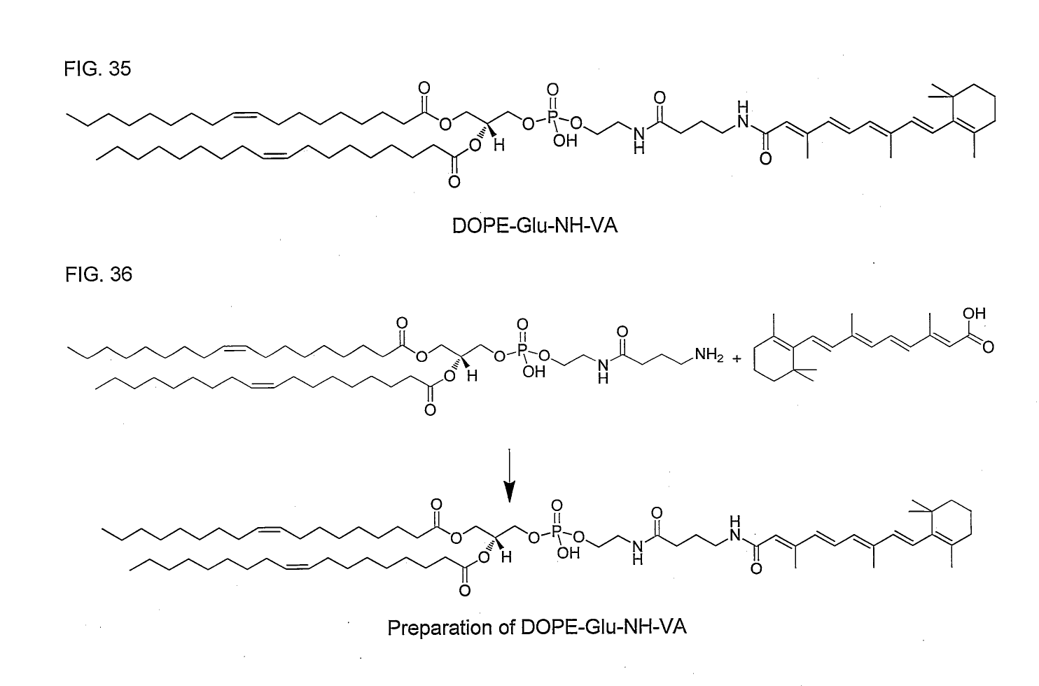

### Preparation of (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)phosphoryl)oxy)propane-1,2-diyl dioleate (DOPE-Glu-NH-VA) (see FIG. 35)

### Preparation of Intermediate 1: (Z)-(2R)-3-(((2-(4-aminobutanamido)ethoxy)(hydroxy)phosphoryl)oxy)propane-1,2-diyl dioleate

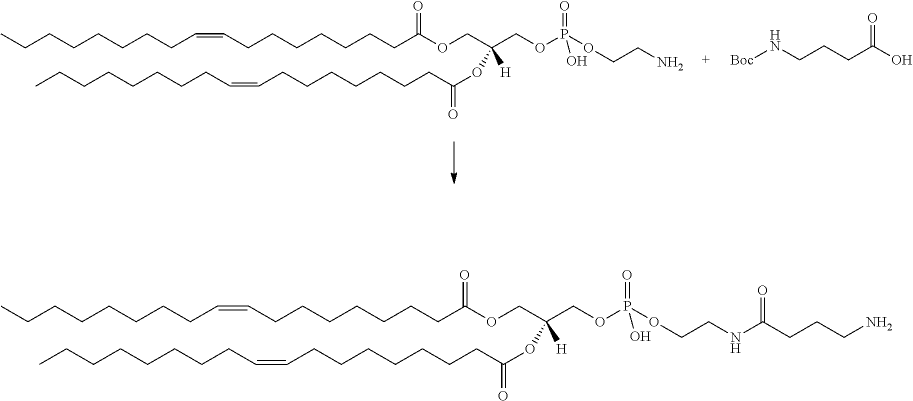 [SMILES: CCCCCCCC/C=C\CCCCCCCC(=O)OC[C@H](COP(=O)(O)OCCN)OC(=O)CCCCCCC/C=C\CCCCCCCC ; CC(C)(C)OC(=O)NCCCC(=O)O ; CCCCCCCC/C=C\CCCCCCCC(=O)OC[C@H](COP(=O)(O)OCCNC(=O)CCCN)OC(=O)CCCCCCC/C=C\CCCCCCCC]

1,2-Dioleoyl-sn-glycero-3-phosphoethanolamine (2500 mg, 3.36 mmol), Boc-GABA-OH (751 mg, 3.70 mmol) and N,N,N′,N′-Tetramethyl-O-(7-azabenzotriazol-1-yl)uronium hexafluorophosphate (1531 mg, 4.03 mmol) were dissolved in a DMF/chloroform (25 mL, 1:1 mixture). N,N-Diisopropylethylamine (880 μL, 5.05 mmol) was added and the mixture was allowed to stir at room temperature overnight under a blanket of argon. The reaction mixture was diluted with ˜200 mL H₂O and product was extracted with dichloromethane (3×100 ml). The product was washed with ˜75 mL pH 4.0 PBS buffer, dried organics with sodium sulfate, filtered and concentrated. Material was then purified via silica gel chromatography with a dichloromethane/methanol gradient, and concentrated to yield colorless oil (2.01 g, 64%). The product was verified by NMR. Material was then taken up in 30 mL of 2 M HCl/diethyl ether. Reaction was allowed to stir at room temperature in a H₂O bath. After 2 hours, the solution was concentrated to yield (Z)-(2R)-3-(((2-(4-aminobutanamido)ethoxy)(hydroxy)phosphoryl)oxy)propane-1,2-diyl dioleate.

### Preparation of DOPE-Glu-NH-VA: (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)phosphoryl)oxy)propane-1,2-diyl dioleate (see FIG. 36)

(Z)-(2R)-3-(((2-(4-aminobutanamido)ethoxy)(hydroxy)phosphoryl)-oxy)propane-1,2-diyl dioleate (1200 mg, 1.45 mmol), retinoic acid (500 mg, 1.66 mmol) and N,N,N′,N′-Tetramethyl-O-(7-azabenzotriazol-1-yl)uronium hexafluorophosphate (689 mg, 1.81 mmol) was suspended in DMF/chloroform (10 mL, 1:1 mixture). N,N-Diisopropylethylamine (758 μL, 4.35 mmol) was added. The round bottom flask was flushed with argon and covered with aluminum foil. Reaction mixture was stirred at room temperature for 4 hours, partitioned in dichloromethane (75 mL) and H₂O (75 mL), extracted with dichloromethane, dried (sodium sulfate), filtered and concentrated. Purification by silica gel chromatography using a dichloromethane/methanol gradient yielded (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)phosphoryl)oxy)propane-1,2-diyl dioleate (292 mg, 18%). The product was characterized by LCMS & NMR. ¹H NMR (400 MHz), δ_{H}: 8.55 (s, 1H), 8.2 (d, 1H), 7.3 (s, 1H), 6.6 (dd, 1H), 6.10-6.27 (m, 5H), 5.5 (t, 1H), 5.31 (s, 4H), 5.1-5.2 (m, 2H), 4.68 (d, 2H), 4.3 (d, 2H), 4.1 (m, 2H), 3.9 (m, 8H), 3.58 (q, 4H), 3.4 (s, 4H), 3.0 (q, 4H), 2.33-2.35 (t, 3H), 2.2-2.3 (m, 7H), 1.9-2.05 (m, 17H), 1.85 (s, 3H), 1.69 (s, 3H), 1.5-1.65 (m, 6H), 1.4-1.5 (m, 2H), 1.18-1.38 (m, ˜40H), 1.01 (s, 3H), 0.84-0.88 (m, 12H). MS: m/z 1112.44 (M+H⁺).

### Example 8

### DSPE-PEG550-VA

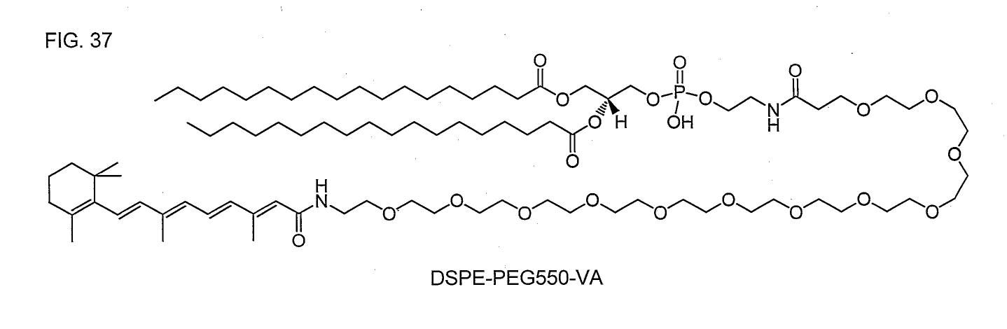

### Preparation of (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate (DSPE-PEG550-VA) (see FIG. 37)

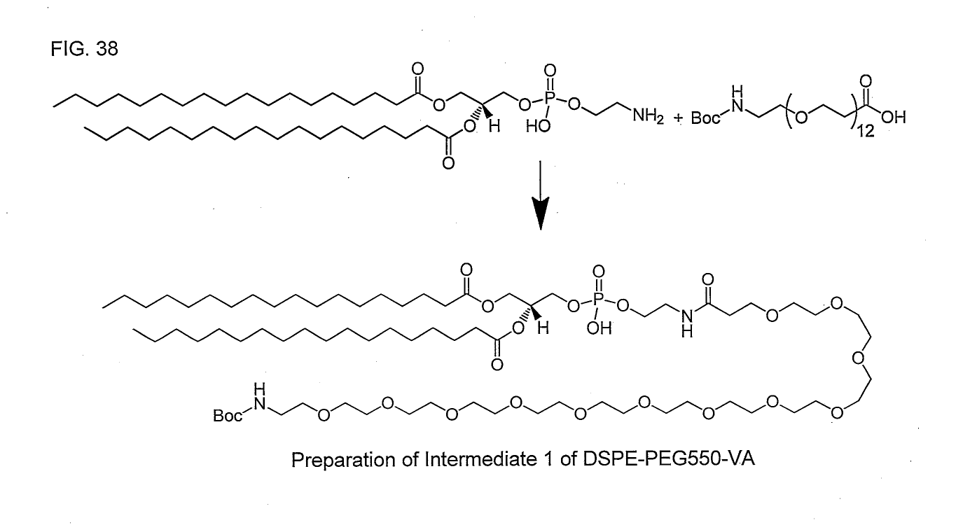

### Preparation of Intermediate 1: (2R)-3-((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate (see FIG. 38)

1,2-Distearoyl-sn-glycero-3-phosphoethanolamine (200 mg, 0.267 mmol), t-Boc-N-amido-dPEG₁₂-acid (211 mg, 0.294 mmol) and N,N,N′,N′-Tetramethyl-O-(7-azabenzotriazol-1-yl)uronium hexafluorophosphate (122 mg, 0.320 mmol) were dissolved in a chloroform/methanol/H₂O (6 mL, 65:35:8) in a 20 mL scintillation vial flushed with argon. N,N-Diisopropylethylamine (116 μL, 0.668 mmol) was added. Reaction was allowed to stir at 25° C. for 4 hours and concentrated. Material was then purified via silica gel chromatography with a dichloromethane/methanol gradient to yield (2R)-3-((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate as an oil (252 mg, 65%).

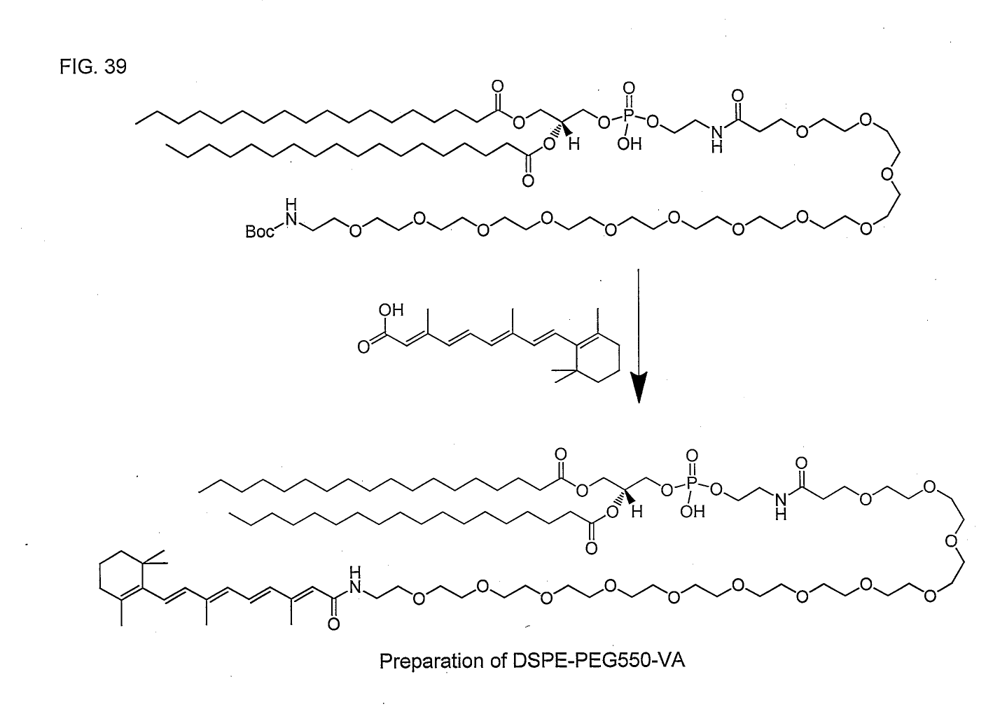

### Preparation of DSPE-PEG550-VA: (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate (see FIG. 39)

(2R)-3-((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate (252 mg, 0.174 mmol) was dissolved in diethyl ether (5 mL) Reaction was placed in a H₂O bath at room temperature. 2 M HCl/diethyl ether (2 mL, 4 mmol) was added and the mixture was allowed to stir for approximately 1 hour. Afterwards, solvent and excess HCl were removed in vacuo. Suspended material in 2 mL N,N-Dimethylformamide in a round bottom flask flushed with argon. Retinoic acid (57.5 mg, 0.191 mmol), N,N,N′,N′-Tetramethyl-O-(7-azabenzotriazol-1-yl)uronium hexafluorophosphate (79 mg, 0.209 mmol) and N,N-Diisopropylethylamine (106 μL, 0.609 mmol) were added. The material did not fully dissolve thus added more chloroform/methanol/H₂O (1 mL, 65:35:8 v:v:v mixture) to get reaction homogeneous. After 3.5 hours, the reaction mixture was concentrated. Material was then purified via silica gel chromatography with a dichloromethane/methanol gradient to yield (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate as a tan solid (210 mg, 74%). Verified product by NMR & LCMS. ¹H NMR (400 MHz), δ_{H}: 8.6 (s, 1H), 8.25 (d, 1H), 6.8-6.9 (dd, 1H), 6.3-6.4 (m, 1H), 6.12-6.25 (dd, 5H), 5.71 (s, 1H), 5.18 (m, 2H), 4.33 (dd, 2H), 4.13 (m, 2H), 3.95 (m, 2H), 3.74 (m, 8H), 3.63 (s, ˜48H), 3.0 (q, 2H), 2.5 (t, 3H), 2.35 (s, 3H), 2.25 (t, 8H), 1.97 (m, 7H), 1.7 (3, 3H), 1.5 (m, 2H), 1.36 (m, 12H), 1.23 (m, ˜56H), 1.01 (s, 6H), 0.86 (t, 12H). MS: m/z 1630.28 (M+H⁺).

### Example 9

### DSPE-PEG2000-Glu-VA

### Preparation of DSPE-PEG2000-Glu-VA

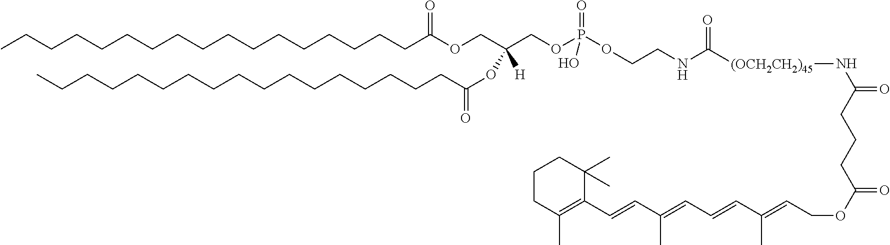

### Preparation of Intermediate 1: 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid

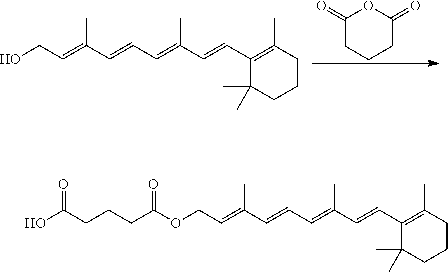 [SMILES: CC1=C(/C=C/C(C)=C/C=C/C(C)=C/CO)C(C)(C)CCC1 ; O=C1CCCC(=O)O1 ; CC1=C(/C=C/C(C)=C/C=C/C(C)=C/COC(=O)CCCC(=O)O)C(C)(C)CCC1]

Glutaric anhydride (115 mg, 1.01 mmol) and retinol (240 mg, 0.838 mmol) were dissolved in dichloromethane (3 mL) in an amber-colored vial. Triethylamine (257 μl, 1.84 mmol) was added and the vial was flushed with argon. Reaction was allowed to stir at room temperature overnight. The reaction mixture was concentrated and then purified via silica gel chromatography with a dichloromethane/methanol gradient to yield 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid as a yellowish oil (700 mg, 78%). Material characterized by NMR.

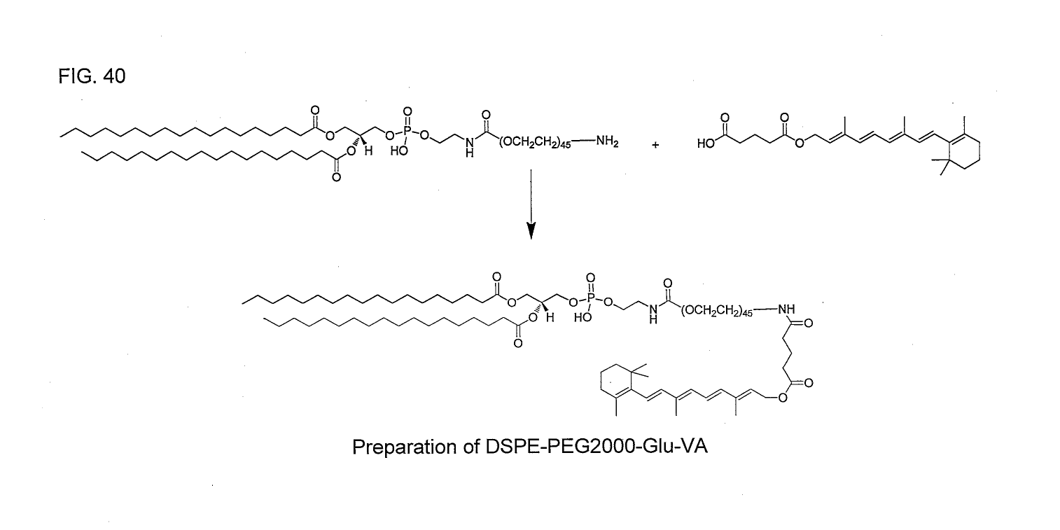

### Preparation of DSPE-PEG2000-Glu-VA (See FIG. 40)

5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid (43 mg, 0.108 mmol), DSPE-PEG2000-NH₂ (250 mg, 0.090 mmol) and N,N,N′,N′-tetramethyl-O-(7-azabenzotriazol-1-yl)uronium hexafluorophosphate (45 mg, 0.117 mmol) were dissolved in N,N-dimethylformamide (2 mL) in an amber-colored scintillation vial flushed with argon gas. N,N-diisopropylethylamine (47 μL, 0.270 mmol) was added and the reaction was allowed to stir overnight at room temperature, then purified via silica gel chromatography with a dichloromethane/methanol gradient to yield yellowish oil (59 mg, 20.7%). Verified product by NMR. ¹H NMR (400 MHz), δ_{H}: 706 (m, 1H), 6.59-6.66 (dd, 1H), 6.06-6.30 (m 5H), 5.56-5.60 (t, 1H), 5.17-5.23 (m, 2H), 4.35-4.42 (dd, 2H), 4.12-4.25 (m, 5H), 3.96-3.97 (m, 6H), 3.79-3.81 (t, 1H), 3.66 (m, ˜180H), 3.51-3.58 (m, 2H), 3.4-3.48 (m, 4H), 3.3-3.38 (m, 2H), 2.25-2.45 (m, 14H), 1.5-2.0 (m, 15H), 1.23-1.32 (m, ˜56H), 1.01 (s, 3H), 0.85-0.88 (t, 12H).

### Example 10

### DOPE-Gly₃-VA

### Preparation of (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate (DOPE-Gly₃-VA) (see FIG. 41)

### Preparation of Intermediate 1: (Z)-(2R)-3-(((2-(2-(2-(2-aminoacetamido)acetamido)acetamido)ethoxy)(hydroxy)phosphoryl)oxy)propane-1,2-diyl dioleate

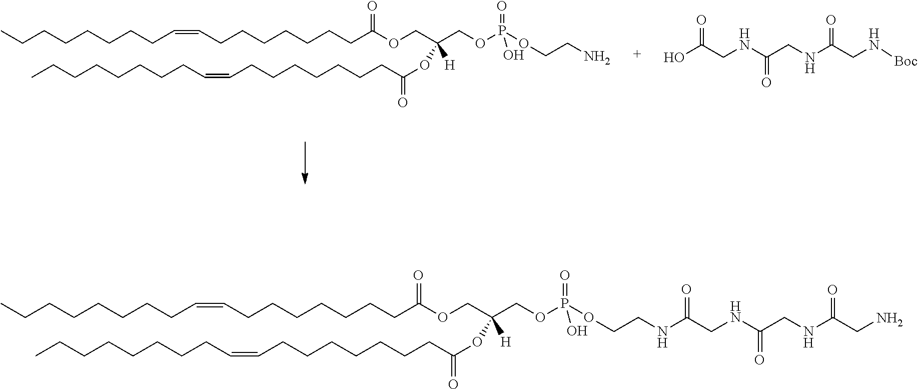 [SMILES: CCCCCCCC/C=C\CCCCCCCC(=O)OC[C@H](COP(=O)(O)OCCN)OC(=O)CCCCCCC/C=C\CCCCCCCC ; CC(C)(C)OC(=O)NCC(=O)NCC(=O)NCC(=O)O ; CCCCCCCC/C=C\CCCCCCCC(=O)OC[C@H](COP(=O)(O)OCCNC(=O)CNC(=O)CNC(=O)CN)OC(=O)CCCCCCC/C=C\CCCCCCCC]

Boc-Gly-Gly-Gly-OH (382 mg, 1.34 mmol) and N,N,N′,N′-Tetramethyl-O-(7-azabenzotriazol-1-yl)uronium hexafluorophosphate (532 mg, 1.4 mmol) were dissolved in DMF (5 mL) N,N-Diisopropylethylamine (488 μL, 2.8 mmol) was added and the mixture was allowed to stir at room temperature for 10-15 minutes. Afterwards, a solution of 1,2-dioleoyl-sn-glycero-3-phosphoethanolamine (833 mg, 1.12 mmol) in chloroform (5 mL) was added and the reaction vessel was flushed with argon. After 16 hours at room temperature, the reaction mixture was concentrated and partitioned between dichloromethane (50 mL) and H₂O (50 mL), extracted with dichloromethane (3×50 mL), dried with sodium sulfate, filtered and concentrated. Material was purified via silica gel chromatography using a dichloromethane/methanol gradient to yield colorless oil residue. To this, 2 M HCl/Diethyl Ether (5 mL) was added and the reaction mixture was allowed to stir in a H₂O bath for approximately 2 hours. The reaction mixture was concentrated and the residue was taken up in dichloromethane (75 mL), washed with saturated sodium bicarbonate solution (75 mL), extracted product with dichloromethane (3×75 mL), dried with sodium sulfate, filtered and concentrated to yield (Z)-(2R)-3-(((2-(2-(2-(2-aminoacetamido)acetamido)acetamido)ethoxy)(hydroxy)phosphoryl)oxy)propane-1,2-diyl dioleate as a semi-solid (765 mg, 90%). Verified by NMR.

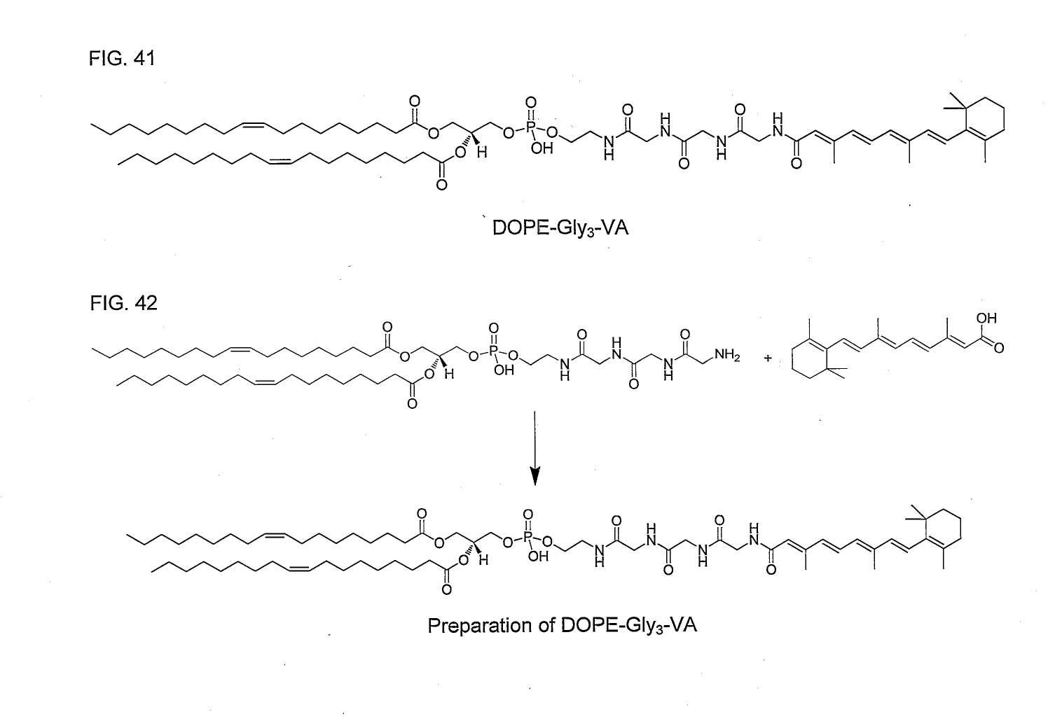

### Preparation of DOPE-Gly₃-VA: (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate (see FIG. 42)

(Z)-(2R)-3-(((2-(2-(2-(2-aminoacetamido)acetamido)acetamido)ethoxy)(hydroxy)phosphoryl)oxy)propane-1,2-diyl dioleate (765 mg, 0.836 mmol), retinoic acid (301 mg, 1.00 mmol), and N,N,N′,N′-Tetramethyl-O-(7-azabenzotriazol-1-yl)uronium hexafluorophosphate (413 mg, 1.09 mmol) were suspended in N,N-Dimethylformamide (5 mL) N,N-Diisopropylethylamine (437 μL, 2.51 mmol) was added and the reaction vessel was flushed with argon gas. Added chloroform (5 mL) to aid in the solvation of materials. Reaction was allowed to stir for ˜4 hours at room temperature in a round bottom flask covered with aluminum foil. Partitioned material between H₂O (100 mL) and dichloromethane (100 mL) Extracted with dichloromethane (3×100 mL), dried with sodium sulfate, filtered and concentrated. Material was then purified via silica gel chromatography using a dichloromethane/methanol gradient to yield (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate as an orange oil (704 mg, 70%). Verified product by LCMS & NMR. ¹H NMR (400 MHz), δ_{H}: 6.90 (t, 1H), 6.21 (q, 2H), 6.08-6.12 (d, 2H), 5.83 (s, 1H), 5.31 (s, 4H), 5.30 (s, 2H), 4.37 (d, 1H), 4.15 (m, 1H), 3.91 (m, 8H), 3.59 (m, 2H), 3.29 (m, 2H), 3.01 (m, 2H), 2.28 (m, 6H), 1.95-1.98 (m, 12H), 1.44 (s, 3H), 1.5-1.6 (m, 2H), 1.44 (m, 6H), 1.24 (m, ˜48H), 1.00 (s, 6H), 0.86 (t, 3H). MS: m/z 1198.42 (M+H⁺).

### Example 11

### VA-PEG-VA

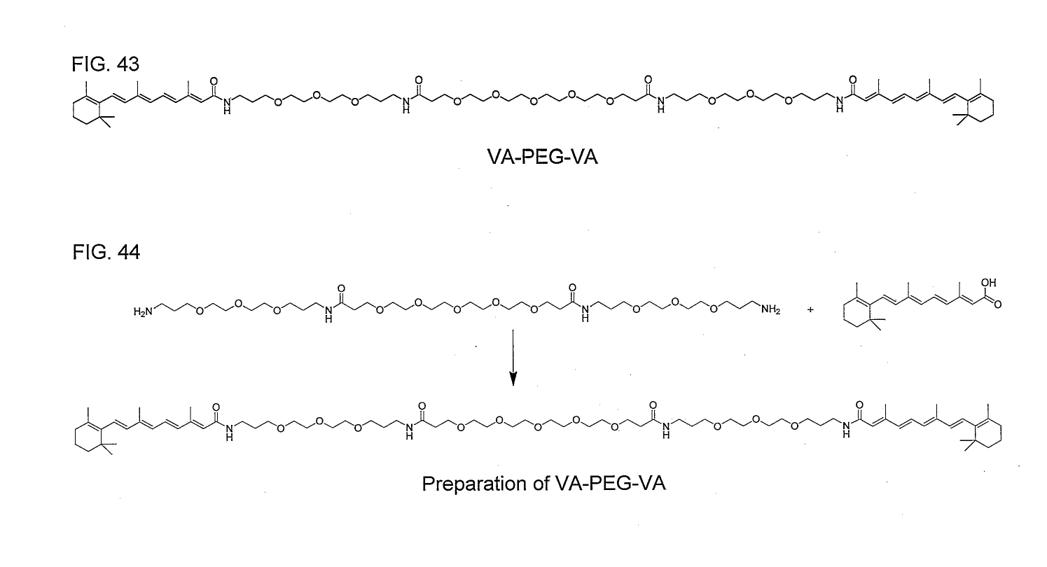

### Preparation of N1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide (VA-PEG-VA) (see FIG. 43)

### Preparation of VA-PEG-VA: N1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide (see FIG. 44)

Retinoic acid (2913 mg, 9.70 mmol), N,N,N′,N′-Tetramethyl-O-(7-azabenzotriazol-1-yl)uronium hexafluorophosphate (3992 mg, 10.50 mmol) and diamido-dPEG₁₁-diamine (3000 mg, 4.04 mmol) were suspended in N,N-dimethylformamide (10 mL) N,N-Diisopropylethylamine (4222 μL, 24.24 mmol) was added and the vessel was flushed with argon. Reaction was allowed to stir at room temperature overnight in a round bottom flask covered with aluminum foil. Next day, partitioned material between ethyl acetate (125 mL) and water (125 mL) Extracted with ethyl acetate (3×125 mL), dried with sodium sulfate, filtered and concentrated. Material was then purified via silica gel chromatography with a dichloromethane/methanol gradient. Pooled fractions and concentrated to yield yellow oil (2900 mg, 54.9%). Verified product by LCMS & NMR. ¹H NMR (400 MHz), δ_{H}: 7.1 (s, 2H), 6.87 (t, 2H), 6.51 (t, 2H), 6.12-6.20 (dd, 8H), 5.66 (s, 2H), 3.6-3.8 (m, ˜44H), 3.4 (q, 4H), 3.3 (q, 4H), 2.46 (t, 4H), 2.32 (s, 6H), 1.9-2.05 (m, 10H), 1.7-1.85 (m, 15H), 1.6 (m, 4H), 1.3-1,5 (m, 6H), 1.01 (s, 12H). QTOF MS: m/z 1306 (M+H⁺).

### Example 12

### VA-PEG2000-VA

### Preparation of (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96, 99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide) (VA-PEG2000-VA)

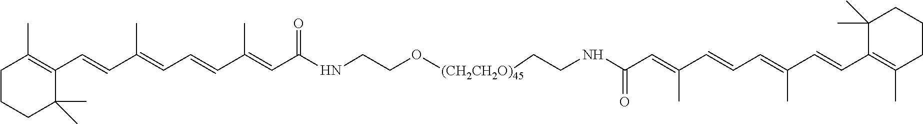

### Preparation of VA-PEG2000-VA: (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96, 99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide)

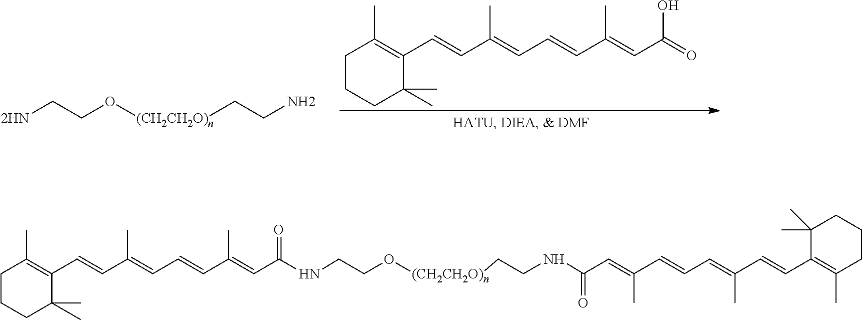

Retinoic acid (109 mg, 0.362 mmol), N,N,N′,N′-Tetramethyl-O-(7-azabenzotriazol-1-yl)uronium hexafluorophosphate (149 mg, 0.392 mmol) and amine-PEG_{2K}-amine (333 mg, 0.151 mmol) were suspended in N,N-Dimethylformamide (3 mL) N,N-Diisopropylethylamine (158 μL, 0.906 mmol) was added and the vessel was flushed with argon. Reaction was allowed to stir at room temperature overnight in a round bottom flask covered with aluminum foil. Next day, partitioned material between ethyl acetate (30 mL) and water (30 mL) Extracted with ethyl acetate (3×30 mL), dried with sodium sulfate, filtered and concentrated. Material was then purified via silica gel chromatography with a dichloromethane/methanol gradient. Pooled fractions and concentrated to yield (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96, 99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide) as a yellow oil (97 mg, 23%). Verified product by LCMS & NMR. ¹H NMR (400 MHz), δ_{H}: 6.85-6.92 (t, 2h), 6.20-6.32 (M, 6H), 6.08-6.12 (d, 4H), 5.72 (s, 2H), 3.55-3.70 (m, ˜180H), 3.4-3.5 (m, 4H), 2.79 (m, 4H), 2.78 (s, 6H), 2.33 (s, 6H), 2.05 (m, 4H), 1.97 (s, 6H), 1.80 (m, 2H), 1.79 (s, 6H), 1.69 (s, 6H), 1.60 (m, 4H), 1.45 (m, 4H), 1.01 (s, 12H). QTOF MS: m/z 2651 (M+H⁺).

### Example 13

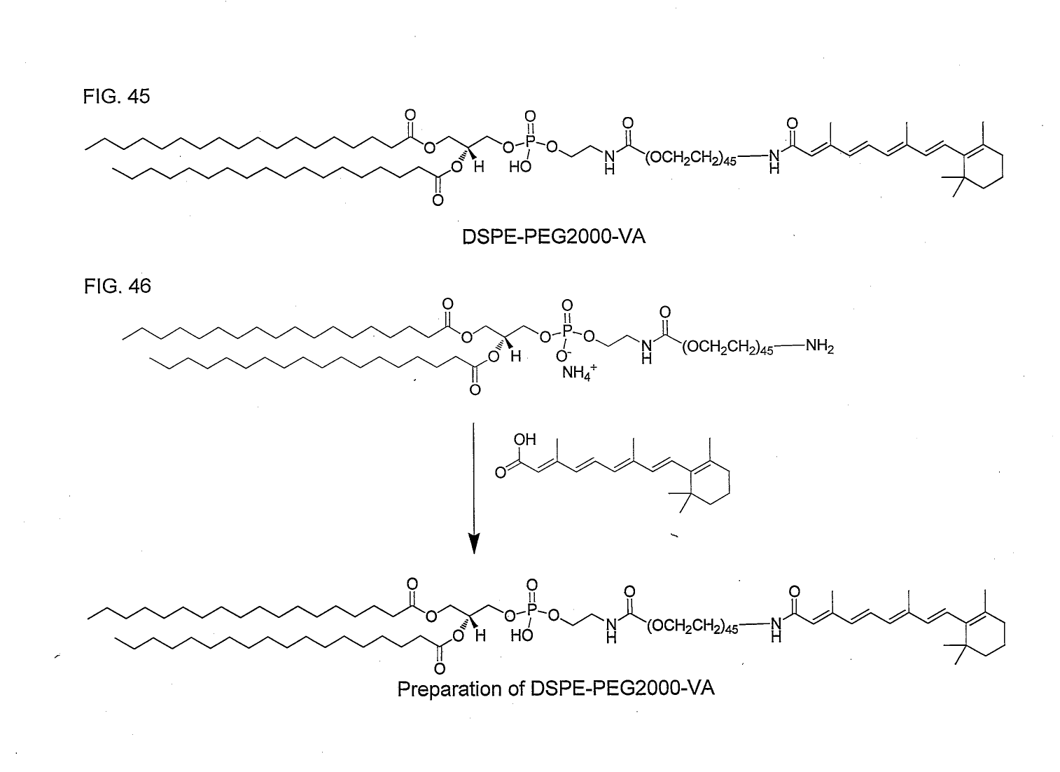

### DSPE-PEG2000-VA (See FIG. 45)

### Preparation of DSPE-PEG2000-VA (see FIG. 46)

DSPE-PEG2000-NH₂ (250 mg, 0.090 mmol), retinoic acid (33 mg, 0.108 mmol) and N,N,N′,N′-Tetramethyl-O-(7-azabenzotriazol-1-yl)uronium hexafluorophosphate (45 mg, 0.117 mmol) were dissolved in N,N-Dimethylformamide. N,N-Diisopropylethylamine (47 μL, 0.270 mmol) was added to the mixture. The amber colored scintillation vial was flushed with argon and allowed to stir 3 days at room temperature. Material was then purified silica gel chromatography using a dichloromethane/methanol gradient. Pooled fractions and concentrated to yield DSPE-PEG2000-VA as a yellow oil (245 mg, 89%). Verified product by NMR. ¹H NMR (400 MHz), δ_{H}: 6.86 (dd, 1H), 6.25 (m, 1H), 6.09-6.21 (dd, 4H), 5.71 (s, 1H), 5.1-5.2 (m, 1H), 4.3-4.4 (d, 1H), 4.1-4.2 (m, 3H), 3.85-4.0 (m, 4H), 3.8 (t, 1H), 3.5-3.75 (m, ˜180H), 3.4-3.5 (m, 8H), 3.3 (m, 2H), 2.35 (s, 3H), 2.26 (m, 4H), 1.70 (s, 3H), 1.55-1.65 (m, 6H), 1.47 (m, 2H), 1.23 (s, ˜60H), 1.01 (s, 6H), 0.85 (t, 6H).

### Example 14

### diVA-PEG-diVA, Also Known as “DIVA”

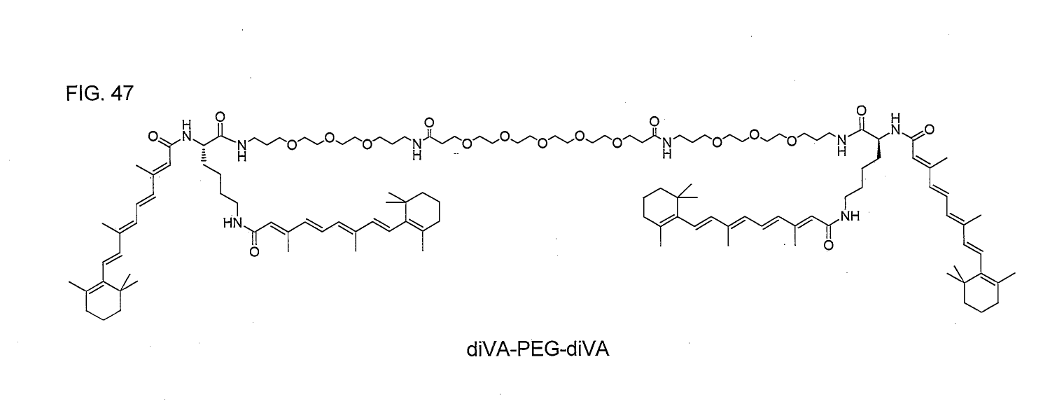

### Preparation of N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide (diVA) (see FIG. 47)

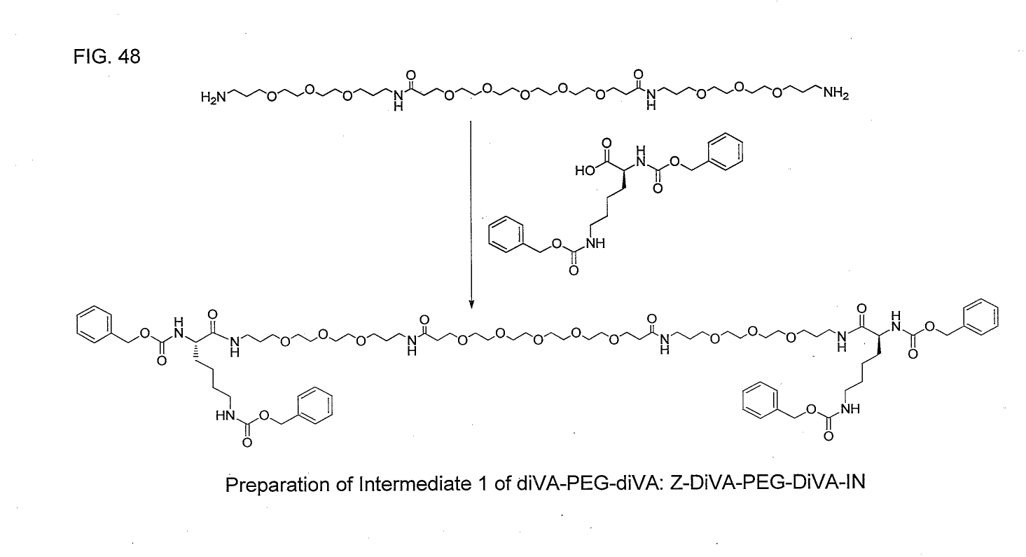

### Preparation of Intermediate 1: tetrabenzyl ((5S,57S)-6,22,40,56-tetraoxo-11,14,17,25,28,31,34,37, 45,48,51-undecaoxa-7,21,41,55-tetraazahenhexacontane-1,5,57,61-tetrayl)tetracarbamate, also known as Z-DiVA-PEG-DiVA-IN (see FIG. 48)

A 1 L reaction flask cooled to 5-10° C. was purged with nitrogen and charged with dichloromethane (300 mL), d-PEG-11-diamine (Quanta lot EK1-A-1100-010, 50.0 g, 0.067 mol), Z-(L)-Lys(Z)-OH (61.5 g, 0.15 mol), and HOBt hydrate (22.5 g, 0.15 mol). 4-Methylmorpholine (4-MMP) (15.0 g, 0.15 mol) was added to the suspension and a light exothermic reaction was observed. A suspension of EDC hydrochloride (43.5 g, 0.23 mol) and 4-MMP (20.0 g, 0.20 mol) in dichloromethane (150 mL) was added over a period of 30 minutes, and moderate cooling was required in order to maintain a temperature of 20-23° C. The slightly turbid solution was stirred overnight at ambient temperature, and HPLC indicates completion of reaction. Deionized water (300 mL) was added and after having stirred for 10 minutes, a quick phase separation was observed. The aqueous phase was extracted with dichloromethane (150 mL)—with a somewhat slower phase separation. The combined organic extracts are washed with 6% sodium bicarbonate (300 mL) and dried with magnesium sulphate (24 g). Evaporation from a 40-45° C. water bath under reduced pressure gives 132 g of crude product. A solution of crude product (131 g) in 8% methanol in ethyl acetate in loaded onto a column of Silica Gel 60 (40-63μ), packed with 8% methanol in ethyl acetate. The column was eluted with 8% methanol in ethyl acetate (7.5 L). The fractions containing sufficiently pure product (5.00-7.25 L) was evaporated from a 45° C. water bath under reduced pressure and 83.6 g of purified product. A solution of purified product (83.6 g) in dichloromethane (200 mL) was loaded onto a column of Dowex 650 C (H⁺) (200 g), which has been washed with dichloromethane (250 mL) The column was eluted with dichloromethane (200 mL) The combined product containing fractions (300-400 mL) were dried with magnesium sulphate (14 g) and evaporated from a 45° C. water bath under reduced pressure to yield tetrabenzyl ((5S,57S)-6,22,40,56-tetraoxo-11,14,17,25,28,31,34,37,45,48,51-undecaoxa-7,21,41,55-tetraazahenhexacontane-1,5,57,61-tetrayl)tetracarbamate, also known as Z-DiVA-PEG-DiVA-IN (77.9 g, HPLC purity 94.1%).

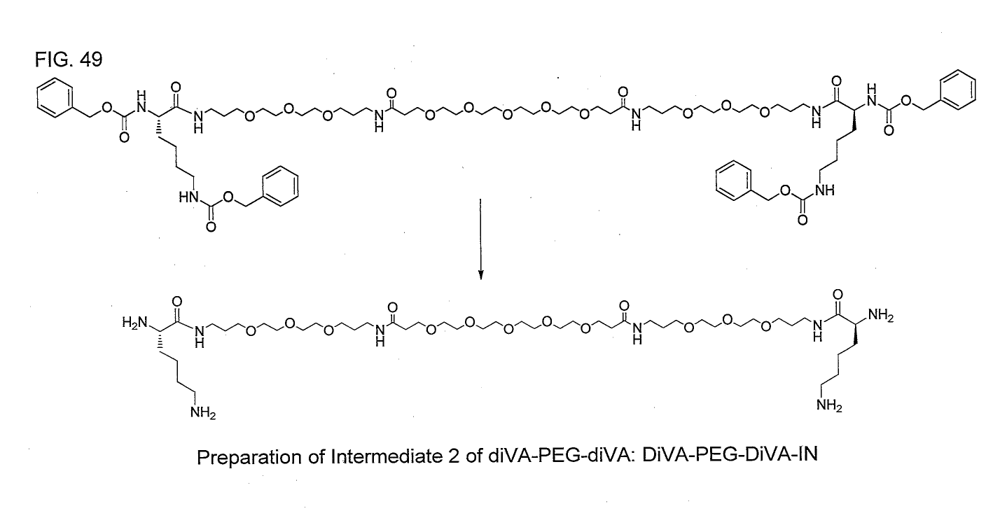

### Preparation of Intermediate 2: N1,N19-bis((S)-16,20-diamino-15-oxo-4,7,10-trioxa-14-azaicosyl)-4,7,10,13,16-pentaoxanonadecane-1,19-diamide, also known as DiVA-PEG-DiVA-IN (see FIG. 49)

A 1 L reaction flask was purged with nitrogen and charged with methanol (600 mL) and Z-DiVA-PEG-DiVA-IN (92.9, 60.5 mmol). The mixture was stirred under nitrogen until a solution was obtained. The catalyst, 10% Pd/C/50%water (Aldrich, 10 g) was added. The mixture was evacuated, and then the pressure was equalized by nitrogen. The mixture was evacuated, and then the pressure was equalized by hydrogen. Ensuring a steady, low flow of hydrogen over the reaction mixture, the stirrer was started. Hydrogenation was continued in a flow of hydrogen for one hour. The system was then closed, and hydrogenation was continued at ˜0.1 bar for one hour. The mixture was evacuated and then re-pressurized to ˜0.1 bar with hydrogen. After another hour of hydrogenation, the mixture was evacuated and then re-pressurized to 0.1 bar with hydrogen. Stirring under hydrogen was continued for 15 hours after which time no starting material could be detected by HPLC. The mixture was evacuated, and then the pressure was equalized by nitrogen. The mixture was evacuated, and then the pressure was equalized by nitrogen. The reaction mixture was then filtered on a pad of celite 545. The filter cake was washed with methanol (100 mL) The combined filtrate was concentrated, finally at 45° C. and at a pressure of less than 50 mbar. Toluene (100 mL) was added and the resulting mixture was again concentrated finally at 45° C. and at a pressure of less than 40 mbar to yield N1,N19-bis((S)-16,20-diamino-15-oxo-4,7,10-trioxa-14-azaicosyl)-4,7,10,13,16-pentaoxanonadecane-1,19-diamide, also known as DiVA-PEG-DiVA-IN (63.4 g), as an oil that solidifies upon standing.

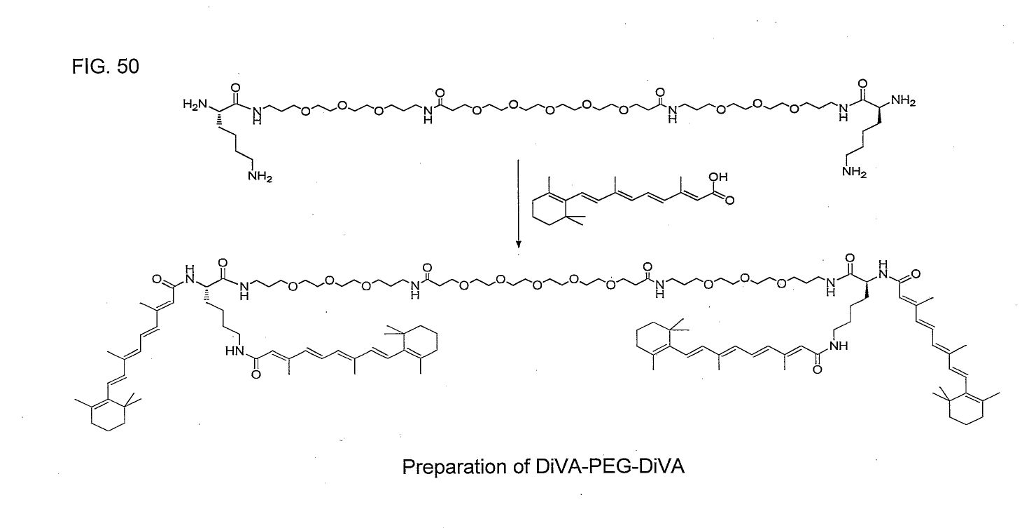

### Preparation of DiVA-PEG-DiVA: N1,N19-bis((S,23E,25E,27E,29E)-16-((2E,4E,6E,8E)-3,7-dimethyl-9-(2,6,6-trimethylcyclohex-1-en-1-yl)nona-2,4,6,8-tetraenamido)-24,28-dimethyl-15,22-dioxo-30-(2,6,6-tri-methylcyclohex-1-en-1-yl)-4,7,10-trioxa-14,21-diazatriaconta-23,25,27,29-tetraen-1-yl)-4,7,10,13,16-pentaoxanonadecane-1,19-diamide (see FIG. 50)

A 2 L reactor was filled with argon and charged with dichloromethane (500 mL), DiVA-PEG-DiVA-IN (52.3 g, 52.3 mmol), retinoic acid (70.6 g, 235 mmol) and 4-N,N-dimethylaminopyridine (2.6 g, 21.3 mmol). The mixture was stirred under argon until dissolved (˜20 minutes). Keeping the temperature of the reaction at 10-20° C., 1-ethyl-3-(3-dimethylaminopropyl)carbodiimide) (EDCI) (70.6 g, 369 mmol) was added portion wise over a period of 10-15 minutes (the reaction was slightly exothermic for the first 30-60 minutes). The reactor was covered with aluminium foil and the mixture was stirred at 18-21° C. for 15-20 hours. Butylated hydroxytoluene (BHT) (25 mg) was added and the reaction mixture was then poured onto aqueous 6% sodium hydrogen carbonate (500 mL) while keeping an argon atmosphere over the mixture. The organic phase was separated. The aqueous phase was washed with dichloromethane (50 mL) The combined organic phase was dried with of magnesium sulphate (150 g) under inert atmosphere and protected from light. The drying agent was filtered off (pressure filter preferred) and the filter cake was washed with dichloromethane (500 mL) The filtrate was concentrated by evaporation at reduced pressure using a water bath of 35-40° C. The oily residue was added toluene (150 mL) and evaporated again to yield a semi-solid residue of 210 g. This residue was dissolved in dichloromethane (250 mL) and applied onto a column prepared from silica gel 60 (1.6 kg) and 0.5% methanol in dichloromethane) (4 L). The column was eluted with dichloromethane (7.2 L), 2), 3% methanol in dichloromethane (13 L), 5% methanol in dichloromethane (13 L), 10% methanol in dichloromethane (18 L). One 10 L fraction was taken, and then 2.5 L fractions were taken. The fractions, protected from light were sampled, flushed with argon and sealed. The fractions taken were analyzed by TLC (10% methanol in dichloromethane, UV). Fractions holding DiVA-PEG-DiVA were further analyzed by HPLC. 5 Fractions<85% pure (gave 32 g of evaporation residue) were re-purified in the same manner, using only 25% of the original amounts of silica gel and solvents. The fractions >85% pure by HPLC were combined and evaporated at reduced pressure, using a water bath of 35-40° C. The evaporation residue (120 g) was re-dissolved in dichloromethane (1.5 L) and slowly passed (approximately 1 hour) through a column prepared from ion exchanger Dowex 650C, H⁺ form (107 g). The column was then washed with dichloromethane (1 L). The combined eluate (3277.4 g) was mixed well and a sample (25 mL, 33.33 g) was evaporated, finally at room temperature and a pressure of <0.1 mBar to afford 0.83 g of a foam. From this figure the total amount of solid material was thus calculated to a yield of 80.8 g (72.5%). The remaining 3.24 kg of solution was concentrated to 423 g. 266 g of this solution was concentrated further to yield a syrup and then re-dissolved in abs. ethanol (200 mL) Evaporation at reduced pressure, using a water bath of 35-40° C., was continued to yield a final ethanol solution of 94.8 g holding 50.8 g (53.6% w/w) of N1,N19-bis((S,23E,25E,27E,29E)-16-((2E,4E,6E,8E)-3,7-dimethyl-9-(2,6,6-trimethylcyclohex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide, also known as DiVA-PEG-DiVA, also known as “DIVA”. Characterized by NMR & QTOF. ¹H NMR (400 MHz), δ_{H}: 7.07 (t, 2H), 7.01 (t, 2H), 6.87-6.91 (m, 4.0H), 6.20-6.24 (m, 10H), 6.10-6.13 (m, 8H), 5.79 (s, 2H), 5.71 (s, 2H), 4.4 (q, 2H), 3.70 (t, 6H), 3.55-3.65 (m, ˜34H), 3.59 (t, 6H), 3.4 (m, 2H), 3.25-3.33 (m, 10H), 3.16 (m, 2H), 2.44 (t, 4H), 2.33 (s, 12H), 1.97-2.01 (m, 12H), 1.96 (s, 6H), 1.7-1.9 (m, 12H), 1.69 (s, 12H), 1.5-1.65 (m, 12H), 1.35-1.5(m, 24H), 1.01 (s, 24H). QTOF MS ESI+: m/z 2128 (M+H⁺).

### Example 15

### DOPE-VA

### Preparation of (Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate (DOPE-VA)

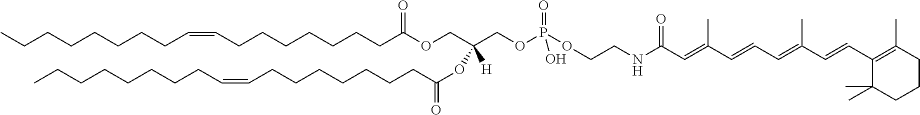 [SMILES: CCCCCCCC/C=C\CCCCCCCC(=O)OC[C@H](COP(=O)(O)OCCNC(=O)/C=C(C)/C=C/C=C(C)/C=C/C1=C(C)CCCC1(C)C)OC(=O)CCCCCCC/C=C\CCCCCCCC]

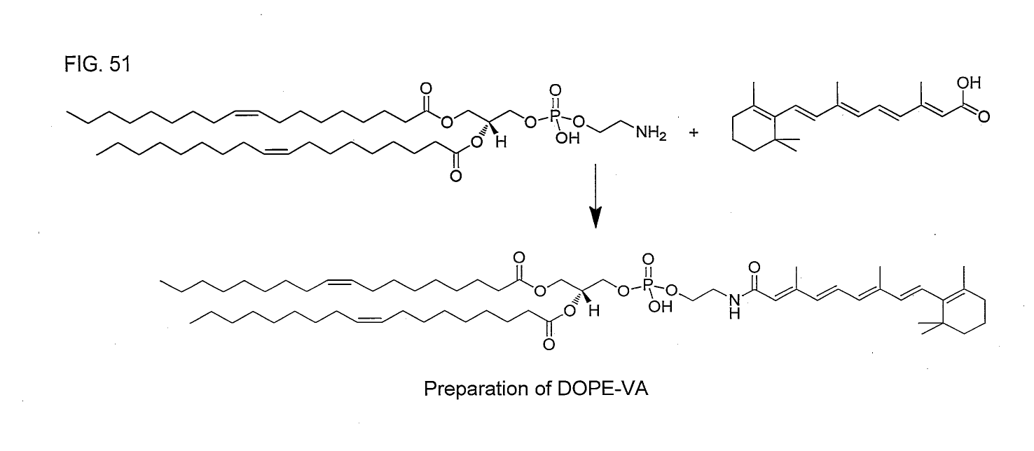

### Preparation of DOPE-VA: (Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate (see FIG. 51)

To a solution of retinoic acid (250 mg, 0.83 mmol) in diethyl ether stirring (20 mL) at −78° C., a solution of (diethylamino)sulfur trifluoride (130 μl, 0.90 mmol) in cold ether (20 mL) was added through a syringe. The reaction mixture was taken out of the cold bath and the stirring was continued at room temperature for an additional 2 hr. At the end, the solvent was removed by rotary evaporation. The residue was redissolved chloroform (50 mL) in the presence of solid Na₂CO₃(50 mg). To this solution was added 1,2-dioleoyl-sn-glycero-3-phosphoethanolamine (600 mg, 0.81 mmol) and the reaction mixture was stirred at room temperature for an additional 24 hrs. The solvent was removed by rotary evaporation. The residue was purified by silica gel chromatography with a dichloromethane/methanol gradient to yield Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate (240 mg, 28%). 1H NMR (400 MHz, CDCl₃) δ 0.87 (t, 6H, CH₃), 1.01 (s, 6H, CH₃) 1.20-1.40 (m, 40H, CH₂), 1.40-1.60 (m, 8H, CH₂), 1.70 (s, 3H, CH₃—C═C), 1.80-2.10 (m, 8H), 2.32 (m, 4H, CH₂C(═O)), 3.50 (m, 2H), 3.92-4.18 (m, 5H), 4.35 (m, 2H), 5.20 (m, 1H, NHC(═O)), 5.31 (m, 4H, CH═CH), 5.80-6.90 (m, 6H, CH═CH).

### Example 16

### DC-VA

### Preparation of (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate (DC-VA)

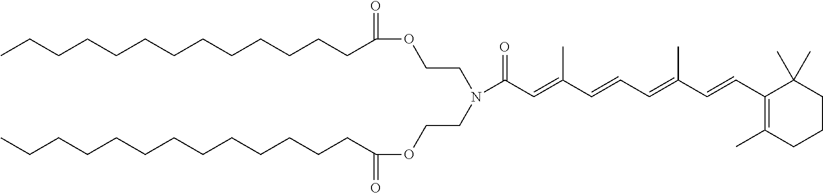 [SMILES: CCCCCCCCCCCCCC(=O)OCCN(CCOC(=O)CCCCCCCCCCCCC)C(=O)/C=C(C)/C=C/C=C(C)/C=C/C1=C(C)CCCC1(C)C]

### Preparation of DC-VA: (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl) ditetradecanoate

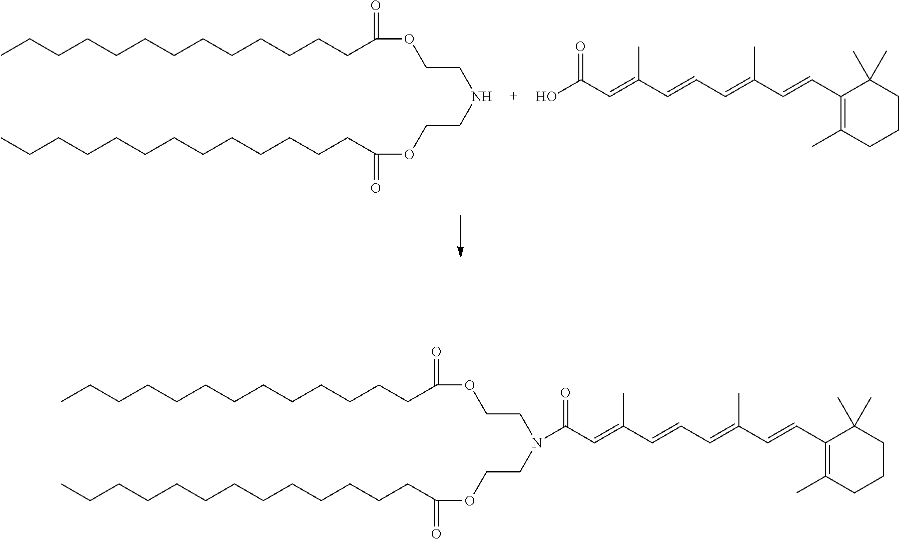

To a solution of retinoic acid (600 mg, 2.0 mmol) in diethyl ether (25 mL) stirring at −78° C., a solution of (diethylamino)sulfur trifluoride (0.3 ml, 2.1 mmol) in 5 mL of cold ether was added through a syringe. The reaction mixture was taken out of the cold bath and the stirring was continued at room temperature for an additional 1 hr. After the solvent was removed by rotary evaporation, the residue was re-dissolved in dichloromethane (20 mL) in the presence of 2 solid Na₂CO₃ (25 mg). To this solution was added the azanediylbis(ethane-2,1-diyl)ditetradecanoate (1.05 g, 2.0 mmol), and the reaction mixture was stirred at room temperature for an additional 24 hrs. The reaction mixture was diluted with dichloromethane (50 mL) and was dried over MgSO₄. After the solvent was removed by rotary evaporation, the residue was purified by silica gel chromatography with a dichloromethane/methanol gradient to yield (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate (800 mg, 50%). 1H NMR (400 MHz, CDCl₃) δ 0.87 (t, 6H, CH₃), 1.02 (s, 6H, CH₃) 1.20-1.40 (m, 40H, CH₂), 1.40-1.60 (m, 8H, CH₂), 1.70 (s, 3H, CH₃—C═C), 1.97(s, 3H, CH₃—C═C), 2.05 (m, 2H, CH₂), 2.15(s, 3H, CH₃—C═C), 2.32 (m, 4H, CH₂C(═O)), 3.67 (m, 4H, NCH₂CH₂O), 4.15-4.30 (m, 4H, NCH₂CH₂O), 5.80-6.90 (m, 6H, CH═CH).

### Example 17

### DC-6-VA

### Preparation of ((6-((2E,4E,6E, 8E)-3,7-dimethyl-9-(2,6,6-trimethylcyclohex-1-en-1-yl)nona-2,4,6,8-tetraenamido)hexanoyl)azanediyl)bis(ethane-2,1-diyl)ditetradecanoate (DC-6-VA)

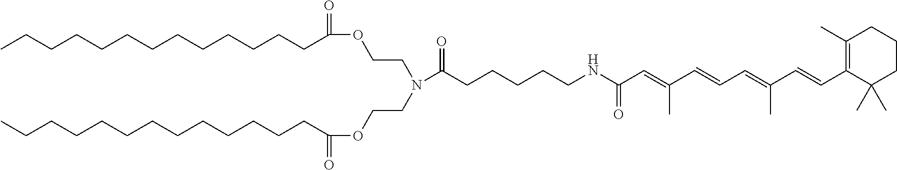 [SMILES: CCCCCCCCCCCCCC(=O)OCCN(CCOC(=O)CCCCCCCCCCCCC)C(=O)CCCCCNC(=O)/C=C(C)/C=C/C=C(C)/C=C/C1=C(C)CCCC1(C)C]

### Preparation of Intermediate 1: ((6-aminohexanoyl)azanediyl)bis(ethane-2,1-diyl)ditetradecanoate TFA Salt

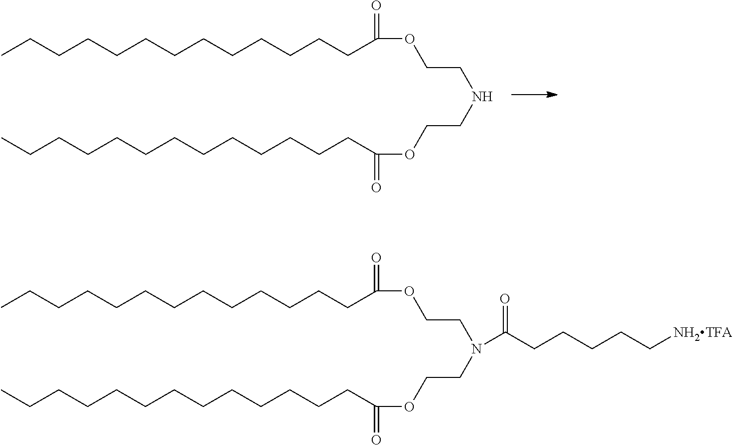

A mixture of azanediylbis(ethane-2,1-diyl)ditetradecanoate (2.5 g, 4.8 mmol), Boc-amino caproic acid (1.3 g, 5.6 mmol), N,N′-dicyclohexylcarbodiimide (1.3 g, 6.3 mmol) and N,N-diisopropylethylamine (2.6 mL, 0.015 mmol) were dissolved in pyridine (40 mL) The solution was stirred at 60° C. for overnight. The mixture was diluted with dichloromethane (50 mL) and washed with saline (3×50 mL) After being concentrated by rotary evaporation, the residue was treated with trifluoroacetic acid/dichloromethane (100 mL, 1:1). The mixture was concentrated and was re-dissolved in dichloromethane (50 mL) and washed with saline (3×50 mL) The organic layer was isolated and concentrated to yield ((6-aminohexanoyl)azanediyl)bis(ethane-2,1-diyl)ditetradecanoate TFA salt (1.5 g, 33%).

### Preparation of DC-6-VA: ((6-((2E,4E,6E,8E)-3,7-dimethyl-9-(2,6,6-trimethylcyclohex-1-en-1yl)nona-2,4,6,8-tetraenamido)hexanoyl)azanediyl)bis(ethane-2,1-diyl)

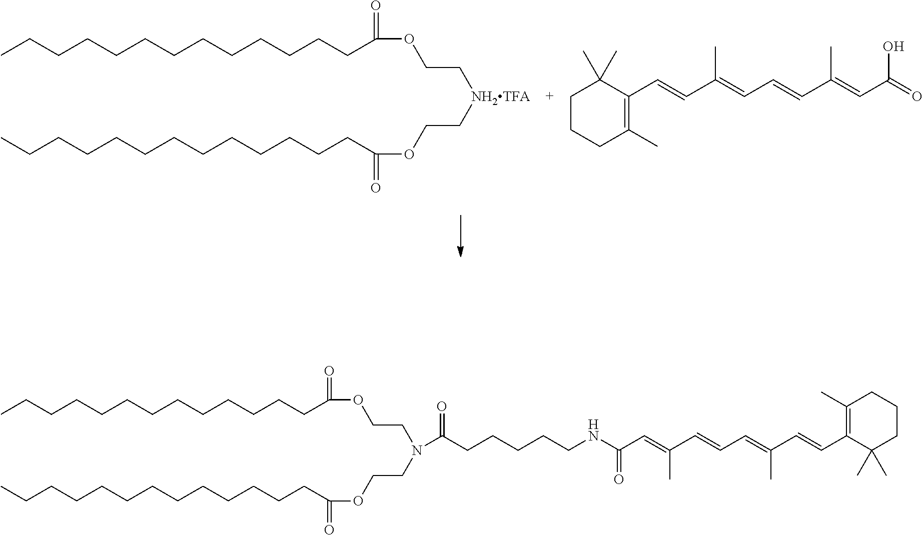

To a solution of retinoic acid (800 mg, 2.67 mmol) in diethyl ether (40 mL) stirring at −78° C., a solution of (diethylamino)sulfur trifluoride (0.4 mL, 22.80 mmol) in cold ether (7 mL) was added through a syringe. The reaction mixture was taken out of the cold bath and the stirring was continued at room temperature for an additional 1 hr. After the solvent was removed by rotary evaporation, the residue was re-dissolved in dichloromethane (25 mL) in the presence of solid Na₂CO₃ (40 mg). To this solution was added the ((6-aminohexanoyl)azanediyl)bis(ethane-2,1-diyl)ditetradecanoate TFA salt (1.5 g, 1.6 mmol) and the reaction mixture was stirred at room temperature for an additional 24 hrs. The reaction mixture was diluted with dichloromethane (50 mL) and dried over MgSO₄. After the solvent was removed by rotary evaporation, the residue was purified by column chromatography using 5% methanol/dichloromethane as eluent to yield ((6-((2E,4E,6E,8E)-3,7-dimethyl-9-(2,6,6-trimethylcyclohex-1-en-1-yl)nona-2,4,6,8-tetraenamido)hexanoyl)azanediyl)bis(ethane-2,1-diyl) (360 mg, 24%). 1H NMR (400 MHz, CDCl₃) δ 0.87 (t, 6H, CH₃), 1.02 (s, 6H, CH₃) 1.20-1.40 (m, 42H, CH₂), 1.40-1.60 (m, 12H, CH₂), 1.70 (s, 3H, CH₃—C═C), 1.97(s, 3H, CH₃—C═C), 2.05 (m, 2H, CH₂), 2.15(s, 3H, CH₃—C═C), 2.32 (m, 6H, CH₂C(═O)), 3.20 (m, 2H, CH₂NHC(═O)), 3.56 (m, 4H, NCH₂CH₂O), 4.15-4.30 (m, 4H, NCH₂CH₂O), 5.10 (m, 1H), 5.80-6.90 (m, 6H, CH═CH).

### Example 18

### In Vitro Evaluation of VA-siRNA-Liposome Formulations for Knockdown Efficiency in LX-2 Cell Line and Rat Primary Hepatic Stellate Cells (pHSCs)

LX2 cells (Dr. S.L. Friedman, Mount Sinai School of Medicine, NY) were grown in DMEM (Invitrogen) supplemented with 10% fetal bovine serum (Invitrogen) at 37° C. in the incubator with 5% CO₂. Cells were trypsinized using TryPLExpress solution (Invitrogen) for 3 min at 37° C. in the incubator. The cell concentration was determined by cell counting in hemocytometer and 3000 cells/well were seeded into the 96-well plates. The cells were grown for 24 h prior to transfection.

Rat primary hepatic stellate cells (pHSCs) were isolated from Sprague-Dawley rats according to the previously published method (Nat. Biotechnol. 2008, 26(4):431-42). pHSCs were grown in DMEM supplemented with 10% fetal bovine serum. Cells were grown up to two passages after isolation before using them for in vitro screening. Cells were seeded at the cell density of 1000 cells/well in 96-well plates and grown for 48 h before using them for transfection.

Transfection with VA-siRNA-Liposome formulations: The transfection method is the same for LX-2 and pHSC cells. The VA-siRNA-Liposome or VA-siRNA-Lipoplex formulations were mixed with growth medium at desired concentrations. 100 μl of the mixture was added to the cells in 96-well plate and cells were incubated for 30 min at 37° C. in the incubator with 5% CO₂. After 30 min, medium was replaced with fresh growth medium after. After 48 h of transfection, cells were processed using Cell-to-Ct lysis reagents (Applied Biosystems) according to the manufacturer's instructions.

Quantitatve (q) RT-PCR for measuring HSP47 mRNA expression: HSP47 and GAPDH TaqMan® assays and One-Step RT-PCR master mix were purchased from Applied Biosystems. Each PCR reaction contained the following composition: One-step RT-PCR mix 5 μl, TaqMan® RT enzyme mix 0.25 μl, TaqMan® gene expression assay probe (HSP47) 0.25 μl, TaqMan® gene expression assay probe (GAPDH) 0.5 μl, RNase-free water 3.25 μl, Cell lysate 0.75 μl, Total volume of 10 μl. GAPDH was used as endogenous control for the relative quantification of HSP47 mRNA levels. Quantitative RT-PCR was performed in ViiA™ 7 realtime PCR system (Applied Biosciences) using an in-built Relative Quantification method. All values were normalized to the average HSP47 expression of the mock transfected cells and expressed as percentage of HSP47 expression compared to mock.

The siRNA referred to in the formulation protocols are double stranded siRNA sequence with 21-mer targeting HSP47/gp46 wherein HSP47 (mouse) and gp46 (rat) are homologs—the same gene in different species:

Rat HSP47-C Double Stranded siRNA Used for In Vitro Assay (Rat pHSCs)

Sense
(SEQ. ID NO. 1)
(5′->3′)GGACAGGCCUCUACAACUATT
Antisense
(SEQ. ID NO. 2)
(3′->5′)TTCCUGUCCGGAGAUGUUGAU.

Cationic Lipid Stock Preparation: Stock solutions of cationic lipids were prepared by combining the cationic lipid with DOPE, cholesterol, and diVA-PEG-DiVA in ethanol at concentrations of 6.0, 5.1 and 2.7 and 2.4 mg/mL respectively. If needed, solutions were warmed up to about 50° C. to facilitate the dissolution of the cationic lipids into solution.

Empty Liposome Preparation: A cationic lipid stock solution was injected into a rapidly stirring aqueous mixture of 9% sucrose at 40±1° C. through injection needle(s) at 1.5 mL/min per injection port. The cationic lipid stock solution to the aqueous solution ratio (v/v) is fixed at 35:65. Upon mixing, empty vesicles formed spontaneously. The resulting vesicles were then allowed to equilibrate at 40° C. for 10 minutes before the ethanol content was reduced to ˜12%.

Lipoplex Preparation: The empty vesicle prepared according to the above method was diluted to the final volume of 1 mM concentration of cationic lipid by 9% sucrose. To the stirring solution, 100 μL of 5% glucose in RNase-free water was added for every mL of the diluted empty vesicle (“EV”) and mixed thoroughly. 150 μL of 10 mg/mL siRNA solution in RNase-free water was then added at once and mixed thoroughly. The mixture was then diluted with 5% glucose solution with 1.750 mL for every mL of the EV used. The mixture was stirred at about 200 rpm at room temperature for 10 minutes. Using a semi-permeable membrane with ˜100000 MWCO in a cross-flow ultrafiltration system using appropriately chosen peristaltic pump (e. g. Midgee Hoop, UFP-100-H24LA), the mixture was concentrated to about ⅓ of the original volume (or desired volume) and then diafiltered against 5 times of the sample volume using an aqueous solution containing 3% sucrose and 2.9% glucose. The product was then filtered through a combined filter of 0.8/0.2 micron pore size under aseptic conditions before use.

Formation of non-diVA siRNA containing liposomes: Cationic lipid, DOPE, cholesterol, and PEG conjugated lipids (e.g., Peg-Lipid) were solubilized in absolute ethanol (200 proof) at a molar ratio of 50:10:38:2. The siRNA was solubilized in 50 mM citrate buffer, and the temperature was adjusted to 35-40° C. The ethanol/lipid mixture was then added to the siRNA-containing buffer while stirring to spontaneously form siRNA loaded liposomes. Lipids were combined with siRNA to reach a final total lipid to siRNA ratio of 15:1 (wt:wt) The range can be 5:1 to 15:1, preferably 7:1 to 15:1. The siRNA loaded liposomes were diafiltered against 10× volumes of PBS (pH 7.2) to remove ethanol and exchange the buffer. Final product was filtered through 0.22 μm, sterilizing grade, PES filter for bioburden reduction. This process yielded liposomes with a mean particle diameter of 50-100 nm, PDI<0.2, >85% entrapment efficiency.

Formation of siRNA containing liposomes co-solubilized with diVA: siRNA-diVA-Liposome formulations were prepared using the method described above. diVA-PEG-diVA was co-solubilized in absolute ethanol with the other lipids (cationic lipid, DOPE, cholesterol, and PEG-conjugated lipids at a ratio of 50:10:38:2) prior to addition to the siRNA containing buffer. Molar content of diVA-PEG-diVA ranged from 0.1 to 5 molar ratio. This process yielded liposomes with a mean particle diameter of 50-100 nm, PDI<0.2, >85% entrapment efficiency.

Formation of siRNA containing liposomes with cationic lipids: siRNA-diVA-Liposome formulations and siRNA-Liposome formulations were prepared using the method described above. Cationic lipid can be, for example, DODC, HEDC, HEDODC, DC-6-14, or any combination of these cationic lipids.

Formation of siRNA containing liposomes decorated with diVA: siRNA-Liposome formulations were prepared using the method described above and diluted to a siRNA concentration of 0.5 mg/mL in PBS. Cationic lipid can be DODC, HEDC, HEDODC, DC-6-14, or any combination of these cationic lipids. diVA-PEG-diVA was dissolved in absolute ethanol (200 proof) to a final concentration ranging from 10 to 50 mg/mL An appropriate amount of ethanol solution was added to the siRNA-Liposome solution to yield a final molar percentage between 2 to 10 mol %. Solution was plunged up and down repeatedly with a pipette to mix. diVA-PEG-diVA concentration and ethanol addition volume were adjusted to keep the addition volume >1.0 μL and the final ethanol concentration <3% (vol/vol). Decorated liposomes were then gently shaken at ambient temperature for 1 hr on an orbital shaker prior to in vitro or in vivo evaluation.

### Results

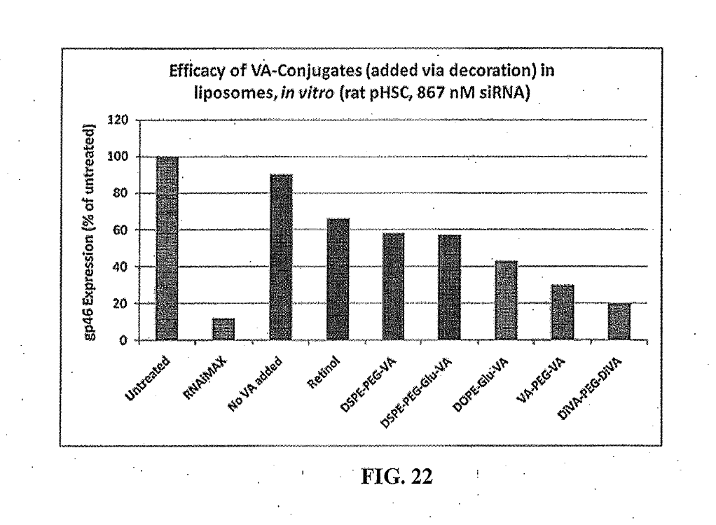

FIG. 22 shows that addition of the VA-conjugate to liposomes via decoration improved the knockdown efficacy of siRNA, enhancing siRNA activity. Peg-Lipid. The dose for all samples was 867 nM siRNA HSP47-C. The results showed that in every instance where a VA-conjugate was added to liposomes, siRNA activity was enhanced compared to liposomes without a retinoid and compared to liposomes decorated with free (non-conjugated) retinol. RNAiMAX was a commercial transfection reagent.

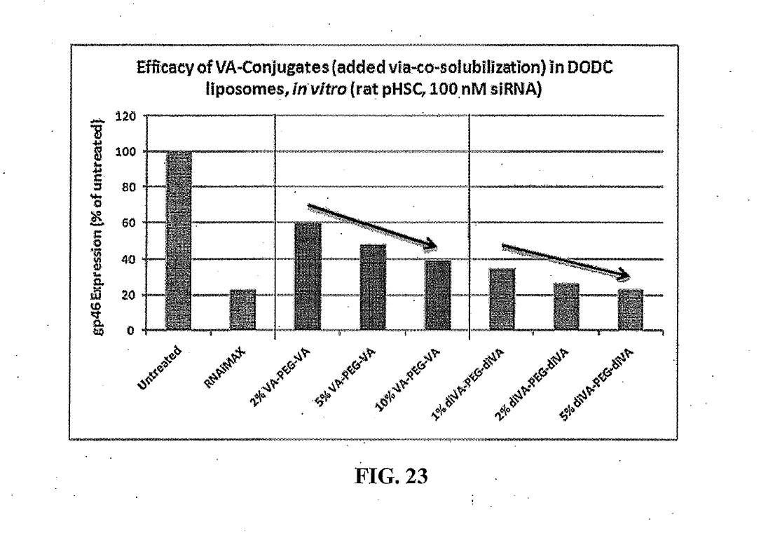

FIG. 23 shows that addition of VA-conjugates to liposomes via co-solubilization improves knockdown efficacy of siRNA. These were DODC containing liposomes with VA-conjugates added via co-solubilization. The formulation is 50:10:38:2:X, where X=1 to 10 (DODC:DOPE:cholesterol:PEG-Lipid:VA-conjugate, mole ratio). The concentration in every instance was 100 nM siRNA HSP47-C. The results show that addition of VA-conjugates to liposomes via cosolubilization enhances siRNA activity.

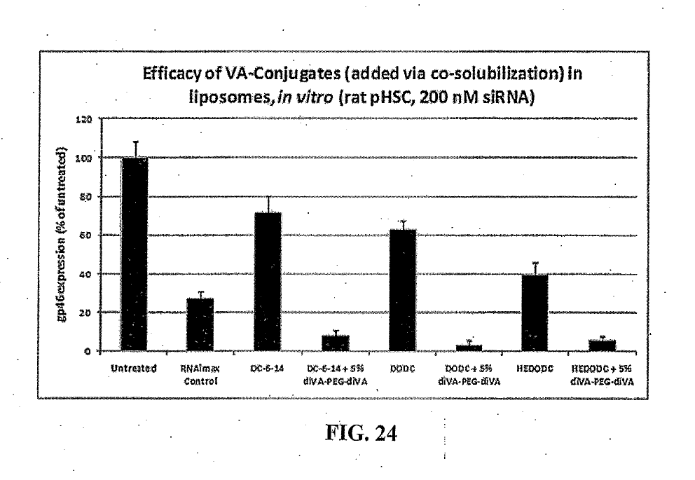

FIG. 24 shows that addition of VA-conjugate to liposomes via co-solubilization dramatically improves the knockdown efficacy of siRNA. Results include three different liposomes, DC-6-14, DODC, HEDODC with VA-conjugates added via co-solubilization. The formulation is the same for all, 50:10:38:2, cationic lipid:DOPE:cholesterol:Peg-Lipid, with only the cationic lipid varying. The concentration of siRNA is 200 nM siRNA HSP47-C is the same for all. The results show in that VA-conjugate addition to liposomes having different cationic lipids significantly enhanced siRNA activity, when prepared by co-solubilization.

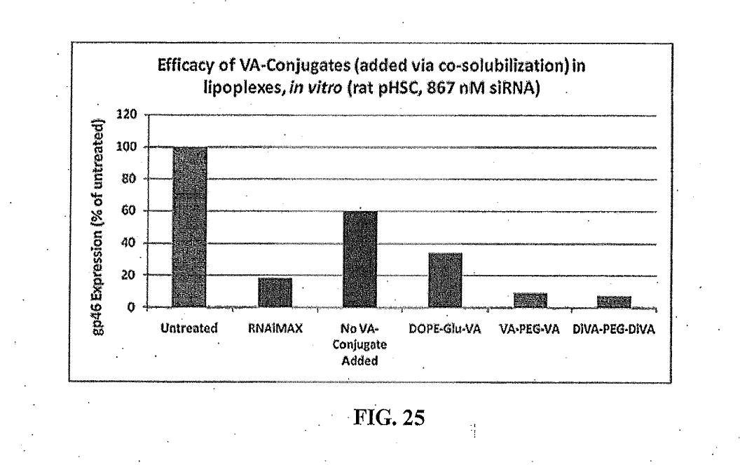

FIG. 25 shows that addition of VA-conjugates to lipoplexes having DC-6-14 cationic lipid via co-solubilization, and siRNA coating the exterior of the liposome enhances siRNA activity. The formulation is a 40% lipoplex formulation, 40:30:30, DC-6-14:DOPE:cholesterol. The concentration for all samples is 867 nM siRNA HSP47-C. The results show that VA-conjugate addition to lipoplexes via co-solubilization enhance siRNA activity.

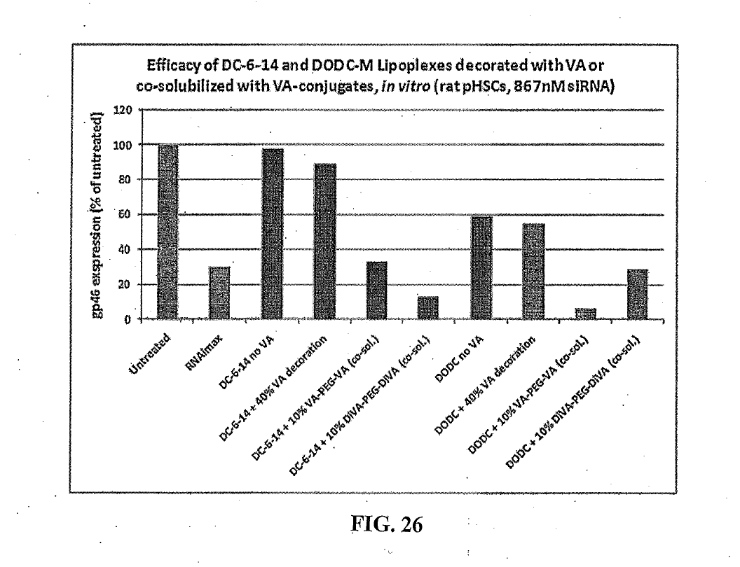

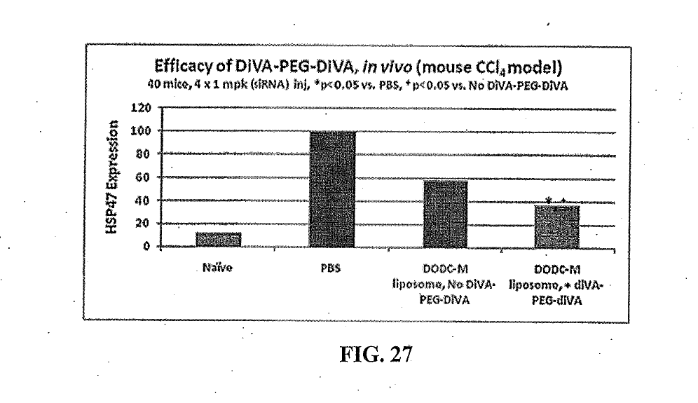

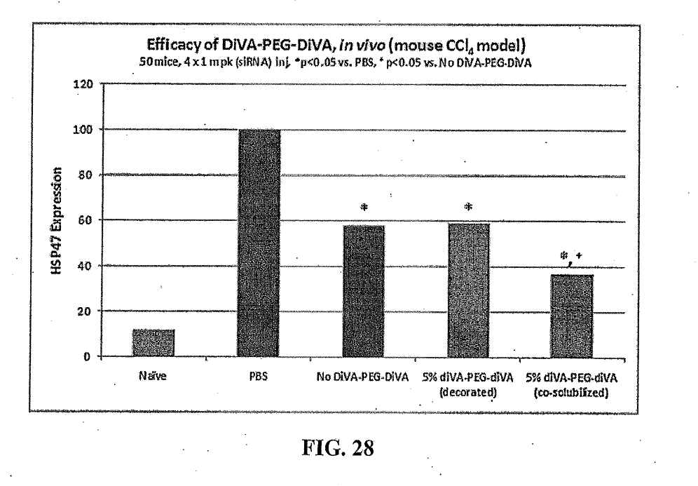

FIG. 26 shows that addition of VA-conjugate to lipoplexes formed via co-solubilization compared to lipoplexes with VA-conjugate added via decoration. These results are from DC-6-14 and DODC lipoplexes. The formulation consists of 40:30:30, DC-6-14:DOPE:cholesterol. The concentration in each sample is 867 nM siRNA HSP47-C. VA-conjugate addition via co-solubilization significantly improves knockdown efficacy in vitro, relative to VA-conjugates added by decoration.

### Example 19

### In Vivo Experiments

Female C57Bl/6 retired breeder mice (Charles River) with a weight range of 24-30 grams were used. Animals were randomly distributed by weight into 10 groups of 10 animals each. All animal procedures were approved by Bio-Quant's IACUC and/or Attending Veterinarian as necessary and all animal welfare concerns were addressed and documented. Mice were anesthetized with Isoflurane and exsanguinated via the inferior vena cava.

Mouse HSP47-C double stranded siRNA used in formulations for in vivo assay (mouse CCl4 model)

Sense
(SEQ. ID NO. 3)
(5′->3′)GGACAGGCCUGUACAACUATT
Antisense
(SEQ. ID NO. 4)
(3′->5′)TTCCUGUCCGGACAUGUUGAU

Upregulation of heat shock protein 47 (HSP47) was induced via intraperitoneal injection of CCl₄ (CCl₄ in olive oil, 1:7 (vol/vol), 1 μL per gram body weight) given every other day for 7 days (day 0, 2, 4, 6). On day 3 mice were treated for 4 consecutive days (day 3, 4, 5, 6) with liposome or lipoplex formulations of the invention or PBS by IV injection into the tail vein. One group of ten mice (naïve) received neither CCl₄ treatment nor IV injection and served as the control group for normal HSP47 gene expression.

### Experimental Timeline

| | Day | | | | | | | |
|---|---|---|---|---|---|---|---|---|
| | 0 | 1 | 2 | 3 | 4 | 5 | 6 | 7 |
| CCl₄ IP Injection | X | X | X | X | X | X | X | |
| Test Article IV | | | | X | X | X | X | |
| Injection | | | | | | | | |
| Sample Collection | | | | | | | | X |
| (n = 10) | | | | | | | | |

On day 7 and approximately 24 hours after final IV injection, all remaining mice were sacrificed and the livers were perfused with PBS prior to collecting liver samples for PCR analysis. An approximate 150 mg sample from each mouse liver was collected and placed in 1.5 mL RNAlater stabilization reagent (Qiagen) and stored at 2-8° C. until analysis. Liver samples were not collected from areas of clear and marked liver damage and/or necrosis.

Total RNA from mouse livers was extracted using RNeasy® columns (Qiagen) according to the manufacturer's protocol. 20 ng of total RNA was used for quantitative RT-PCR for measuring HSP47 expression. HSP47 and GAPDH TaqMan® assays and One-Step RT-PCR master mix were purchased from Applied Biosystems. Each PCR reaction contained the following composition: One-step RT-PCR mix 5 μl, TaqMan® RT enzyme mix 0.25 μl, TaqMan® gene expression assay probe (HSP47) 0.25 μl, TaqMan® gene expression assay probe (GAPDH) 0.5 μl, RNase-free water 3.25 μl, RNA 0.75 μl, Total volume of 10 μl. GAPDH was used as endogenous control for the relative quantification of HSP47 mRNA levels. Quantitative RT-PCR was performed in ViiA™ 7 realtime PCR system (Applied Biosciences) using an in-built Relative Quantification method. All values were normalized to the average HSP47 expression of the naïve animal group and expressed as percentage of HSP47 expression compared to naïve group.

### Example 20

### Synthesis of satDiVA

### Preparation of N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide (satDIVA) (see FIG. 52)

### Preparation of Intermediate 1: 3,7-dimethyl-9-(2,6,6-trimethylcyclohex-1-en-1-yl)nonanoic acid

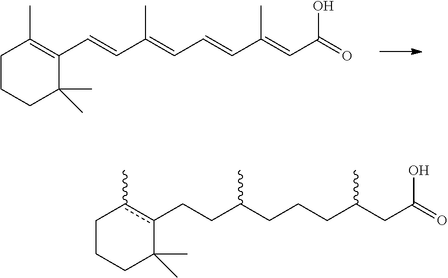

All-trans retinoic acid (2000 mg, 6.66 mmol) was dissolved in hexanes/IPA (3:1, 40 mL) with the aid of sonication. Material was placed in a Parr-shaker bottle and flushed with inert gas. 10% Pd/C (200 mg) was added and the vessel was once again flushed with inert gas. Material was placed on the Parr-Shaker overnight with >70 psi Hydrogen gas. The reaction mixture was then filtered through a pad of celite and concentrated to yield 3,7-dimethyl-9-(2,6,6-trimethylcyclohex-1-en-1-yl)nonanoic acid (2 g).

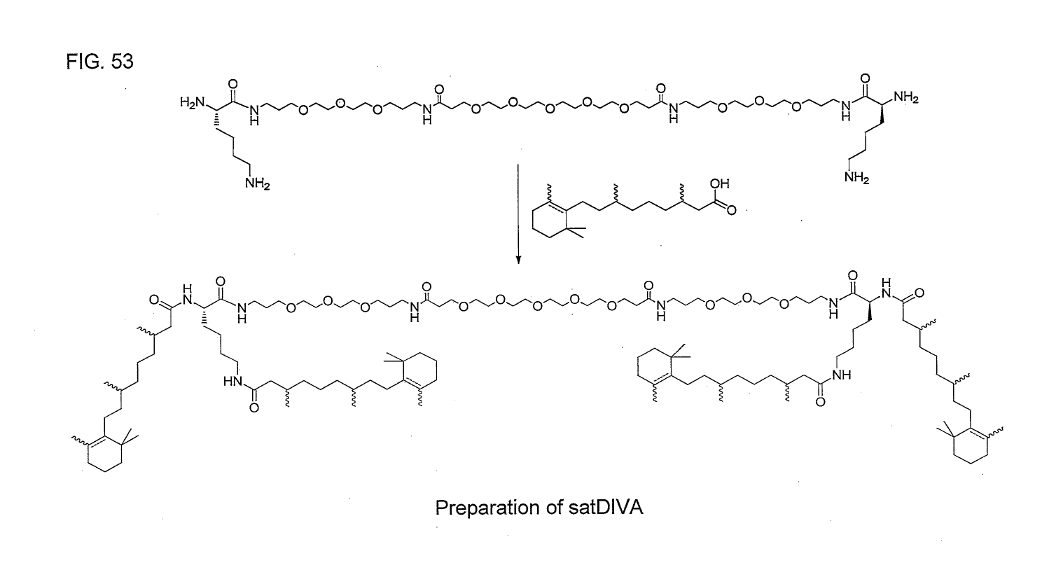

### Preparation of satDIVA: N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide (see FIG. 53)

N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide, also known as satDIVA, was prepared in similar fashion as diva-PEG-diVA from previously described N1,N19-bis((S)-16,20-diamino-15-oxo-4,7,10-trioxa-14-azaicosyl)-4,7,10,13,16-pentaoxanonadecane-1,19-diamide with the substitution of 3,7-dimethyl-9-(2,6,6-trimethylcyclohex-1-en-1-yl)nonanoic acid for all-trans retinoic acid. QTOF MS ESI+: m/z 2161, 2163, 2165 & 2167 (M+H+)

### Example 21

### Synthesis of simDiVA

### Preparation of N1,N19-bis((S)-15,22-dioxo-30-(2,6,6-trimethylcyclohex-1-en-1-yl)-16-(9-(2,6,6-trimethylcyclohex-1-en-1-yl)nonanamido)-4,7,10-trioxa-14,21-diazatriacontyl)-4,7,10,13,16-pentaoxanonadecane-1,19-diamide (simDiVA) (see FIG. 54)

### Preparation of Intermediate 1: 2,6,6-trimethylcyclohex-1-en-1-yl trifluoromethanesulfonate

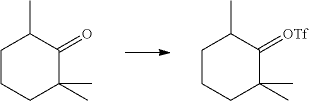

To a solution of 2,2,6-trimethylcyclohexanone in dry THF at −78° C. under nitrogen was added dropwise a 2 M lithium diisopropylamide solution. The mixture was stirred at −78° C. for 3 h. A solution of N-phenyl-bis(trifluoromethanesulfonimide) in THF was then added dropwise (at −78° C.). The reaction flask was packed in dry-ice and stirred overnight. The stirring was continued at room temperature for 3 h under which time all material had dissolved. The reaction mixture was concentrated and the residue was added slowly to hexane (350 mL) under vigorous stirring. The solid material was removed by filtration and washed with hexane (2×50 mL) The filtrate was concentrated and more hexane (150 mL) was added. The solid material was removed by filtration and the filtrate was concentrated. The precipitation was repeated one more time after which the residue was purified by flash chromatography (silica, hexane) to give 2,6,6-trimethylcyclohex-1-en-1-yl trifluoromethanesulfonate as a colorless oil (23.2 g, 60% yield).

### Preparation of Intermediate 2: ethyl 9-(bromozincio)nonanoate

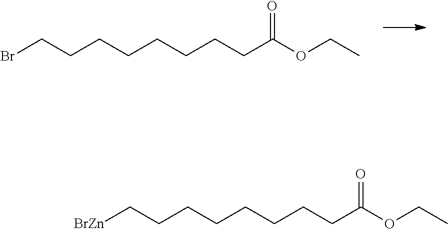

In a dry reaction tube under nitrogen were charged zinc dust (3.70 g, 56.6 mmol), iodine (479 mg, 1.89 mmol) and dry DMA (20 mL) The mixture was stirred at room temperature until the color of iodine disappeared. Ethyl 9-bromononanoate was added, and the mixture was stirred at 80° C. for 4 hours and then at room temperature overnight. (Completion of the zinc insertion reaction was checked by GCMS analysis of the hydrolyzed reaction mixture.) The reaction mixture was used without further treatment in the subsequent step. GCMS m/z 186 [M]+ (ethyl nonanoate).

### Preparation of Intermediate 3: ethyl 9-(2,6,6-trimethylcyclohex-1-en-1-yl)nonanoate

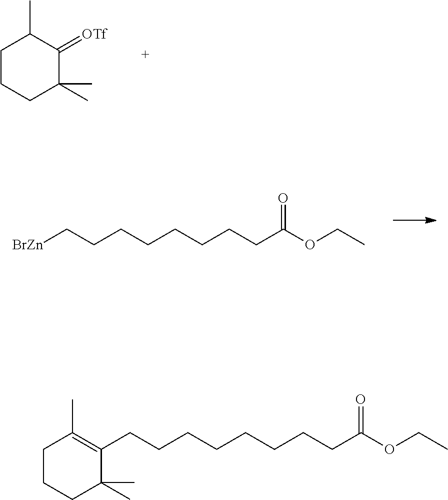

To freshly prepared ethyl 9-(bromozincio)nonanoate (37.7 mmol) in dimethylacetamide under nitrogen in a reaction tube was added 2,6,6-trimethylcyclohex-1-en-1-yl trifluoromethanesulfonate (10.8 g, 39.6 mmol) followed by tetrakis(triphenylphosphine)palladium(0) (872 mg, 0.754 mmol). The tube was sealed and the mixture was stirred at 95° C. for 2 h. The reaction mixture was allowed to cool and was then poured into diethyl ether (100 mL) The upper layer was decanted and the lower layer was washed twice with diethyl ether (2×25 mL) The combined ether layers were washed with sat NH₄Cl and brine, dried (MgSO₄) and concentrated to give crude material (˜12 g). The material was purified by flash chromatography (silica, 0 to 1.5% EtOAc in hexane). The obtained oil was stirred under vacuum for 8 h in order to remove most of the side-product, ethyl nonanoate, and was then purified by a second flash chromatography (silica, 0 to 15% toluene in hexane). The fractions were analyzed by LCMS and GCMS. The purest fractions were collected and concentrated at a temperature below 25° C. to give ethyl 9-(2,6,6-trimethylcyclohex-1-en-1-yl)nonanoate as a colorless oil (6.16 g, 53% yield over two steps). LCMS ESI+ m/z 309 [M+H]+; GCMS m/z 308 [M]+.

### Preparation of Intermediate 4: 9-(2,6,6-trimethylcyclohex-1-en-1-yl)nonanoic acid

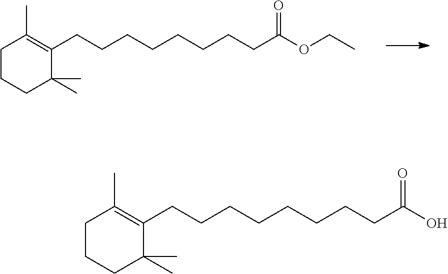 [SMILES: CCOC(=O)CCCCCCCCC1=C(C)CCCC1(C)C ; CC1=C(CCCCCCCCC(=O)O)C(C)(C)CCC1]

To ethyl 9-(2,6,6-trimethylcyclohex-1-en-1-yl)nonanoate (13.2 g, 42.9 mmol) in ethanol (80 mL) was added 4 M KOH (43 mL) The mixture was stirred at room temperature for 1.5 h. Water (350 mL) was added and the solution was washed with teat-butyl methyl ether (2×100 mL) The SimVA, aqueous phase was cooled, acidified with 4 M HCl (˜45 mL) and extracted with pentane (3×100 mL) The combined pentane extracts were washed with water (200 mL), dried (MgSO4), filtered, concentrated and dried under high vacuum. The material was redissolved in pentane (100 mL), concentrated and dried under high vacuum one more time to give 9-(2,6,6-trimethylcyclohex-1-en-1-yl)nonanoic acid as a colorless oil (11.1 g, 92% yield). MS ESI− m/z 279 [M−H]−.

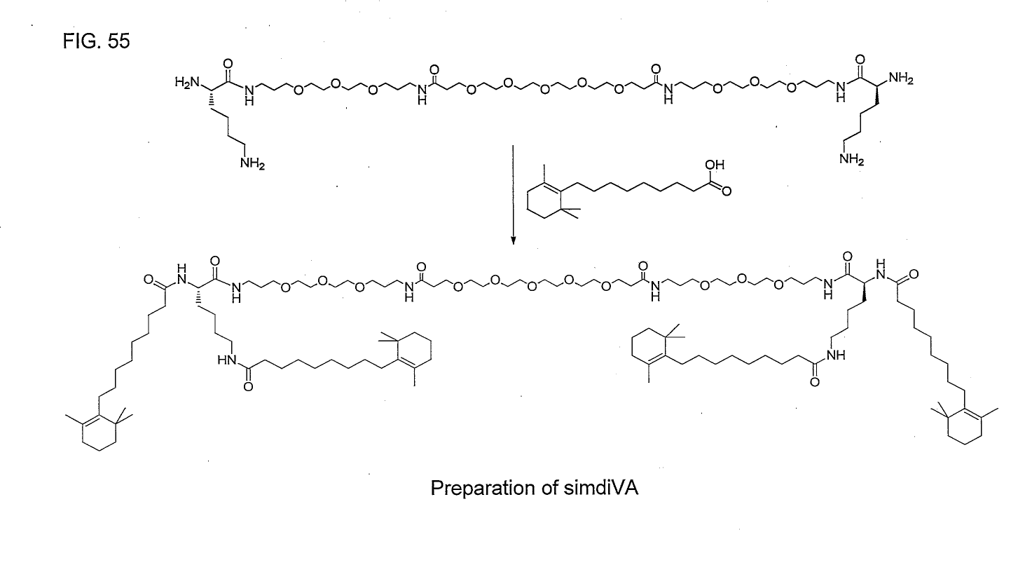

### Preparation of simdiVA: N1,N19-bis((S)-15,22-dioxo-30-(2,6,6-trimethylcyclohex-1-en-1-yl)-16-(9-(2,6,6-trimethylcyclohex-1-en-1-yl)nonanamido)-4,7,10-trioxa-14,21-diazatriacontyl)-4,7,10,13,16-pentaoxanonadecane-1,19-diamide (see FIG. 55)

simDIVA was prepared in similar fashion as diVA from previously described N1,N19-bis((S)-16,20-diamino-15-oxo-4,7,10-trioxa-14-azaicosyl)-4,7,10,13,16-pentaoxanonadecane-1,19-diamide with the substitution of 9-(2,6,6-trimethylcyclohex-1-en-1-yl)nonanoic acid for all-trans retinoic acid. QTOF MS ESI+: m/z 2050 (M+H+)

### Example 22

### Synthesis of DiVA-PEG18

### Preparation of (2E,2′E,2″E,4E,4′E,4″E,6E,6′E,6″E,8E,8′E,8″E)-N,N′,N″-((5R,69R,76E,78E,80E,82E)-77,81-dimethyl-6,68,75-trioxo-83-(2,6,6-trimethylcyclohex-1-en-1-yl)-10,13,16,19,22,25,28,31,34,37,40,43,46,49,52,55,58,61,64-nonadecaoxa-7,67,74-triazatrioctaconta-76,78,80,82-tetraene-1,5,69-triyl)tris(3,7-dimethyl-9-(2,6,6-trimethylcyclohex-1-en-1-yl)nona-2,4,6,8-tetraenamide) (DIVA-PEG18) (see FIG. 56)

(2E,2′E,2″E,4E,4′E,4″E,6E,6′E,6″E,8E,8′E,8″E)-N,N′,N″-((5R,69R,76E,78E,80E,82E)-77,81-dimethyl-6,68,75-trioxo-83-(2,6,6-trimethylcyclohex-1-en-1-yl)-10,13,16,19,22,25,28,31,34,37,40,43,46,49,52,55,58,61,64-nonadecaoxa-7,67,74-triazatrioctaconta-76,78,80,82-tetraene-1,5,69-triyl)tris(3,7-dimethyl-9-(2,6,6-trimethylcyclohex-1-en-1yl)nona-2,4,6,8-tetraenamide), also known as DIVA-PEG18 was prepared in similar fashion as diVA with the substitution of PEG₁₈ diamine for diamido-dPEG₁₁-diamine. LCMS ESI+: m/z 2305 (M+Na).

### Example 23

### Synthesis Of TriVA (see FIG. 57)

### Preparation of Intermediate 1: (S)-methyl 6-(((benzyloxy)carbonyl)amino)-2-((S)-2,6-bis(((benzyloxy)carbonyl)amino)hexanamido)hexanoate

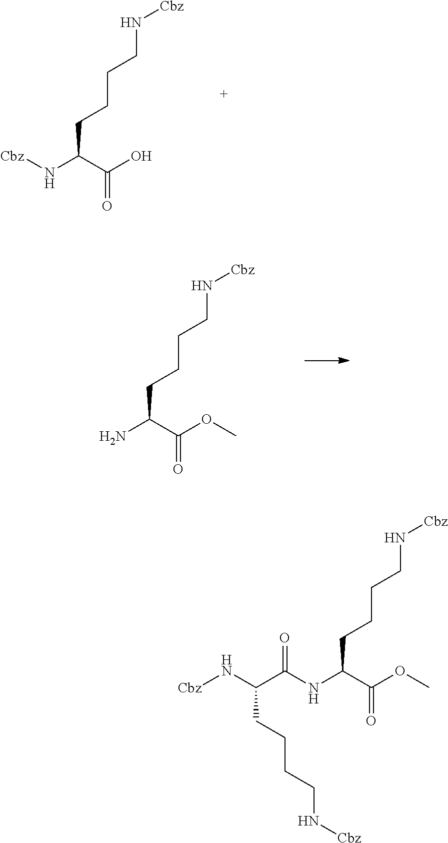 [SMILES: O=C(NCCCC[C@H](NC(=O)OCc1ccccc1)C(=O)O)OCc1ccccc1 ; COC(=O)[C@@H](N)CCCCNC(=O)OCc1ccccc1 ; COC(=O)[C@H](CCCCNC(=O)OCc1ccccc1)NC(=O)[C@H](CCCCNC(=O)OCc1ccccc1)NC(=O)OCc1ccccc1]

A flask was purged with inert gas and H-Lys(Z)-OMe HCl salt (4 g, 12.1 mmol), HOBt hydrate (1.84 g, 13.6 mmol), Z-Lys(Z)-OH (5.64 g, 13.6 mmol) are suspended in dichloromethane (50 mL) NMM (1.5 mL, 13.6 mmol) was added to the suspension and the solution became clear. A suspension EDC HCl salt (4.01 g, 20.9 mmol) and NMM (2.0 mL, 18.2 mmol) in dichloromethane (50 mL) was added over a period of 10 minutes. The reaction was stirred overnight at room temperature, then washed with 1M HCl (100 mL), H₂O (100 mL), saturated bicarbonate solution (100 mL) and saturated brine solution (100 mL) All aqueous washes were back extracted with dichloromethane (50 mL) Dried organics with Na₂SO₄, filtered and concentrated. Material was purified by silica gel chromatography with a dichloromethane/methanol gradient to yield (S)-methyl 6-(((benzyloxy)carbonyl)amino)-2-((S)-2,6-bis(((benzyloxy)carbonyl)amino)hexanamido) hexanoate (6.91 g).

### Preparation of Intermediate 2: (S)-6-(((benzyloxy)carbonyl)amino)-2-((S)-2,6-bis(((benzyloxy)carbonyl)amino)hexanamido)hexanoic acid

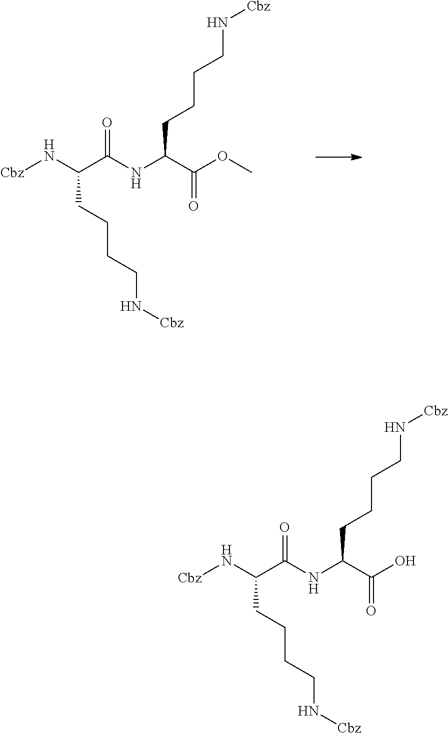 [SMILES: COC(=O)[C@H](CCCCNC(=O)OCc1ccccc1)NC(=O)[C@H](CCCCNC(=O)OCc1ccccc1)NC(=O)OCc1ccccc1 ; O=C(NCCCC[C@H](NC(=O)[C@H](CCCCNC(=O)OCc1ccccc1)NC(=O)OCc1ccccc1)C(=O)O)OCc1ccccc1]

6-(((benzyloxy)carbonyl)amino)-2-((S)-2,6-bis(((benzyloxy)carbonyl)amino)hexanamido)hexanoate (6.91 g, 10 mmol) was dissolved with methanol (50 mL) Added KOH (2.24 g, 40 mmol) and allowed mixture to stir at 35° C. After 2 hours, quenched reaction by adding H₂O (200 mL) and washed mixture with diethyl ether (50 mL) Afterwards, adjusted the pH to ˜2 with 1M HCl acid. Extracted product with dichloromethane (3×100 mL), dried with Na₂SO₄, filtered and concentrated to yield (S)-6-(((benzyloxy)carbonyl)amino)-2-((S)-2,6-bis(((benzyloxy)carbonyl)amino)hexanamido)hexanoic acid (4 g).

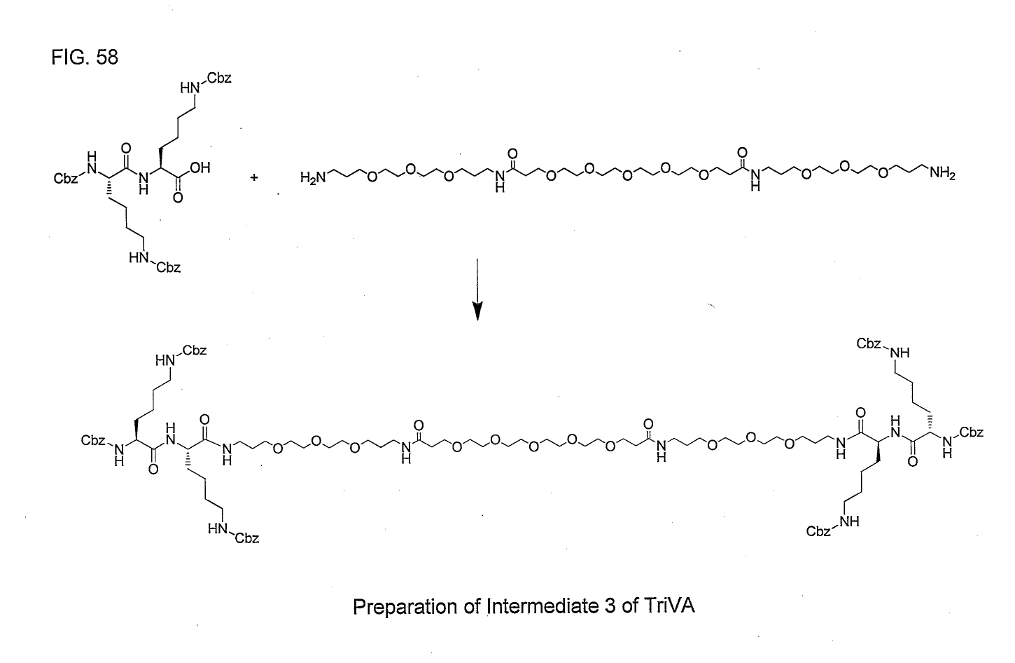

### Preparation of Intermediate 3: (Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide (see FIG. 58)

A round bottom flask was purged with inert gas and diamido-dPEG₁₁-diamine (1 g, 1.35 mmol), (S)-6-(((benzyloxy)carbonyl)amino)-2-((S)-2,6-bis(((benzyloxy)carbonyl)amino)hexanamido)hexanoic acid (2.05 g, 3.03 mmol), HOBt hydrate (409 mg, 3.03 mmol) are suspended in dichloromethane (25 mL) NMM (333 uL, 3.03 mmol) was added to the suspension and the solution became clear. A suspension EDC HCl salt (893 mg, 4.66 mmol) and NMM (445 uL, 4.05 mmol) in dichloromethane (25 mL) was added over a period of 10 minutes. The reaction was allowed to stir overnight at room temperature, then washed with 1M HCl (100 mL), H₂O (100 mL), saturated bicarbonate solution (100 mL) and saturated brine solution (100 mL) All aqueous washes were back extracted with dichloromethane (50 mL) Dried organics with Na₂SO₄, filtered and concentrated. Material was purified by silica gel chromatography with a dichloromethane/methanol gradient to yield (Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide (480 mg).

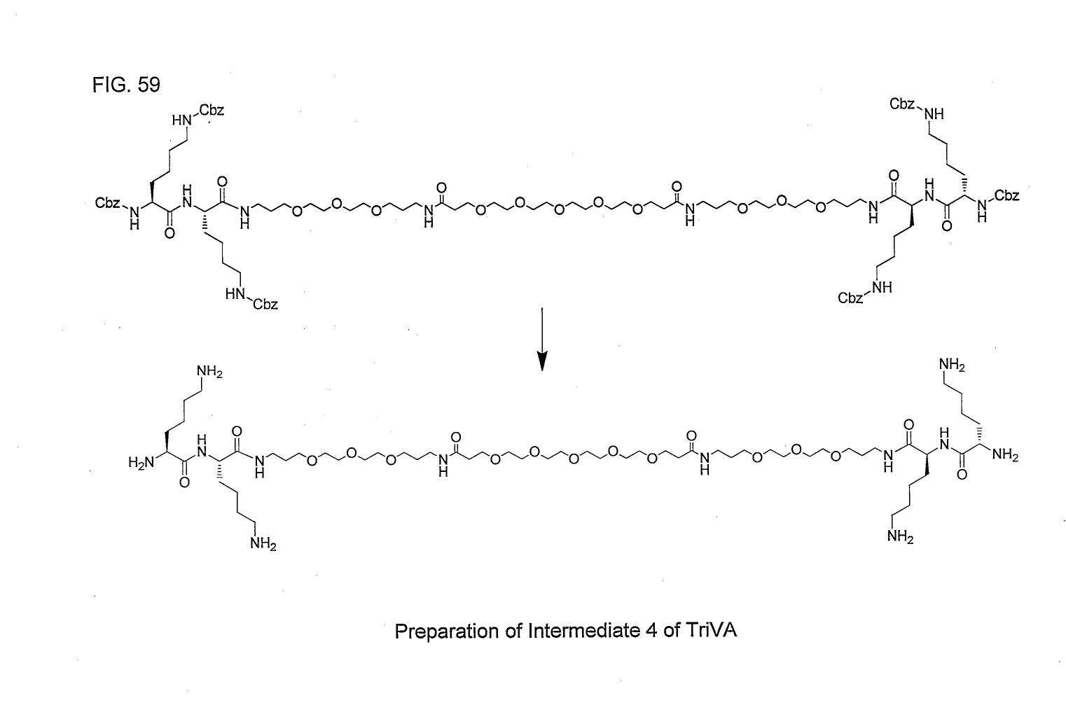

### Preparation of Intermediate 4: N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide (see FIG. 59)

(Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide was dissolved in methanol (30 mL) in a round bottom flask and flushed with an inert gas. 10% Pd/C (135 mg) was added and the flask was once again flushed with inert gas and then all air was removed via vacuum pump. An 8″ H₂ balloon was added and the reaction was allowed to stir at room temperature. After 2 hours, the Pd/C was removed by filtering through a pad of celite washing with methanol, and concentrated to yield N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide (823 mg).

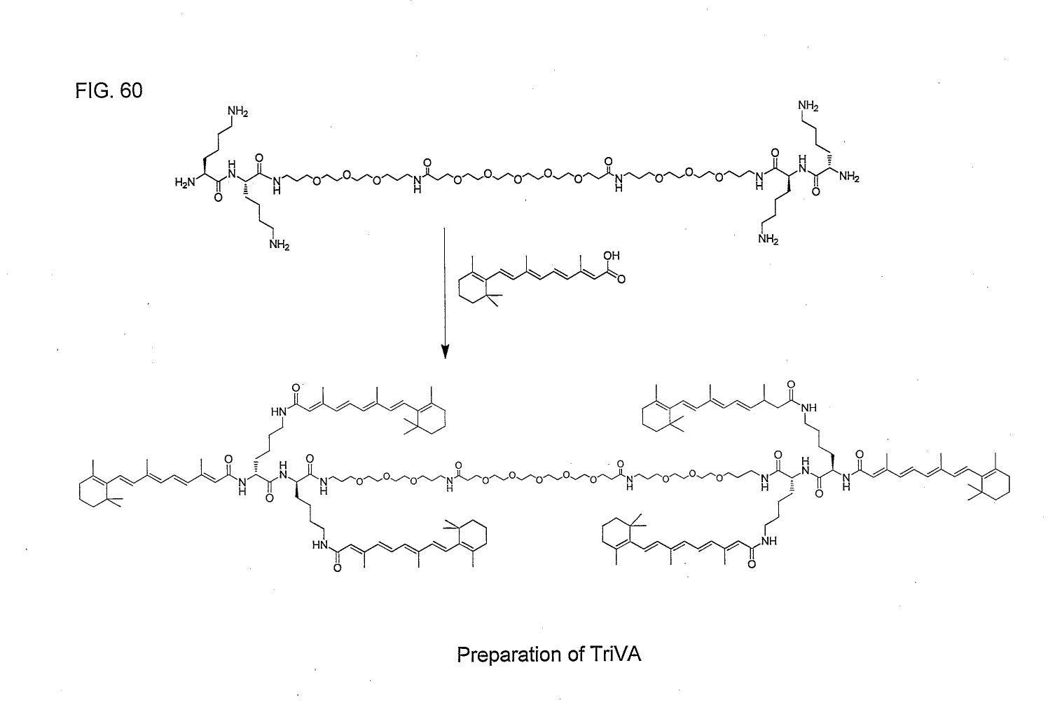

### Preparation of TriVA (see FIG. 60)

N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide was stirred in dichloromethane and DMAP and retinoic acid was added. NMM was added and the solution was stirred in an aluminum foil covered round bottom flask flushed with inert gas at room temperature. A suspension of EDC HCl salt & NMM in dichloromethane (20 mL) was slowly added to reaction over a period of 10 minutes. Reaction was allowed to stir overnight at room temperature. Next day, diluted with dichloromethane to 100 mL Washed with H₂O (100 mL), saturated bicarbonate solution (100 mL) and saturated brine solution (100 mL) All aqueous washes were back extracted with dichloromethane (50 mL) Dried organics with Na₂SO₄, filtered and concentrated. Material was purified by basic alumina chromatography eluating with dichloromethane/ethanol gradient to yield TriVA (780 mg). LCMS ESI+: m/z 2972 (M+Na).

### Example 24

### Synthesis of 4TTNPB

### Preparation of N1,N19-bis((R)-1,8-dioxo-7-(4-((E)-2-(5,5,8,8-tetramethyl-5,6,7,8-tetrahydro-naphthalen-2-yl)prop-1-en-1-yl)benzamido)-1-(4-((E)-2-(5,5,8,8-tetramethyl-5,6,7,8-tetrahydronaphthalen-2-yl)prop-1-en-1-yl)phenyl)-13,16,19-trioxa-2,9-diazadocosan-22-yl)-4,7,10,13,16-pentaoxanonadecane-1,19-diamide (4TTNPB) (see FIG. 61)

N1,N19-bis((R)-1,8-dioxo-7-(4-((E)-2-(5,5,8,8-tetramethyl-5,6,7,8-tetrahydro-naphthalen-2-yl)prop-1-en-1-yl)benzamido)-1-(4-((E)-2-(5,5,8,8-tetramethyl-5,6,7,8-tetrahydronaphthalen-2-yl)prop-1-en-1-yl)phenyl)-13,16,19-trioxa-2,9-diazadocosan-22-yl)-4,7,10,13,16-pentaoxanonadecane-1,19-diamide, also known as 4TTNPB, was prepared in similar fashion as N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide, also known as diVA, from N1,N19-bis((S)-16,20-diamino-15-oxo-4,7,10-trioxa-14-azaicosyl)-4,7,10,13,16-pentaoxanonadecane-1,19-diamide with the substitution of TTNPB for all-trans retinoic acid. LCMS ESI+: m/z 2343 (M+Na).

### Example 25

### Synthesis of 4Myr

### Preparation of N1,N19-bis((R)-15,22-dioxo-16-tetradecanamido-4,7,10-trioxa-14,21-diazapenta-triacontyl)-4,7,10,13,16-pentaoxanonadecane-1,19-diamide (4Myr) (see FIG. 62)

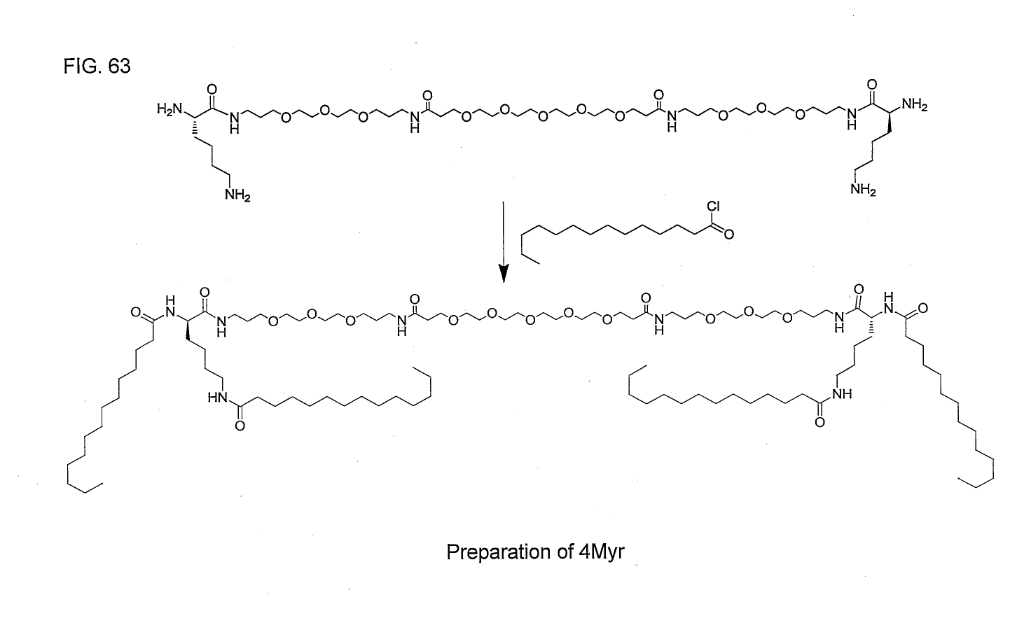

### Preparation of 4Myr: N1,N19-bis((R)-15,22-dioxo-16-tetradecanamido-4,7,10-trioxa-14,21-diaza-penta-triacontyl)-4,7,10,13,16-pentaoxanonadecane-1,19-diamide (see FIG. 63)

N1,N19-bis((S)-16,20-diamino-15-oxo-4,7,10-trioxa-14-azaicosyl)-4,7,10,13,16-pentaoxanonadecane-1,19-diamide (synthesis previously described) was dissolved in dichloromethane and placed in an ice-bath. Myristoyl chloride was added followed by triethylamine. The ice-bath was removed and the reaction was allowed to stir overnight at room temperature under a blanket of inert gas. Next day, diluted with dichloromethane to 100 mL and washed with 1M HCl (75 mL), H₂O (75 mL), saturated bicarbonate solution (75 mL) and saturated brine solution (75 mL). Back extracted all aqueous washes with dichloromethane (25 mL) Dried organics with MgSO₄, filtered and concentrated. Purification by silica gel chromatography with a dichloromethane/methanol gradient yielded N1,N19-bis((R)-15,22-dioxo-16-tetradecanamido-4,7,10-trioxa-14,21-diaza-penta-triacontyl)-4,7,10,13,16-pentaoxanonadecane-1,19-diamide (410 mg). LCMS ESI+: m/z 1841 (M+H).

### Example 26

### Synthesis of DiVA-242

### Preparation of N1,N16-bis((R,18E,20E,22E,24E)-11-((2E,4E,6E,8E)-3,7-dimethyl-9-(2,6,6-trimethyl-cyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide, also known as DIVA-242 (see FIG. 64)

### Preparation of Intermediate 1: di-tert-butyl (10,25-dioxo-3,6,13,16,19,22,29,32-octaoxa-9,26-diazatetratriacontane-1,34-diyl)dicarbamate

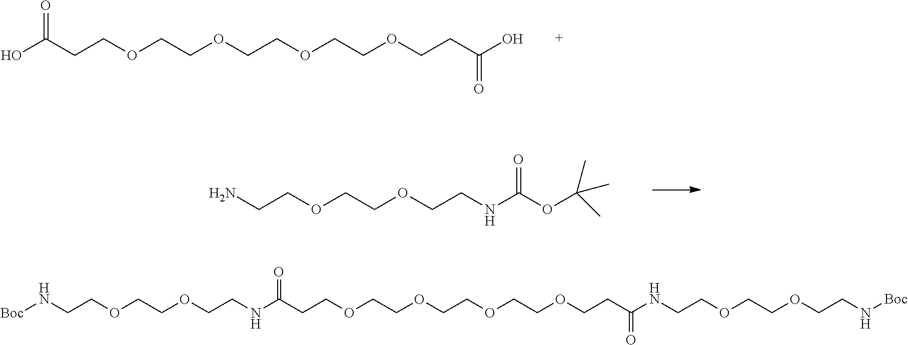 [SMILES: O=C(O)CCOCCOCCOCCOCCC(=O)O ; CC(C)(C)OC(=O)NCCOCCOCCN ; CC(C)(C)OC(=O)NCCOCCOCCNC(=O)CCOCCOCCOCCOCCC(=O)NCCOCCOCCNC(=O)OC(C)(C)C]

A round bottom flask containing dichloromethane (25 mL) was purged with inert gas and Bis-dPeg₄ acid (1000 mg, 3.40 mmol), N-Boc-3,6-dioxa-1,8-octane diamine (1816 uL, 7.65 mmol) and HOBt hydrate (1034 mg, 7.65 mmol) were added. NMM (841 uL, 7.65 mmol) was added to the suspension and the solution became clear. A suspension of EDC HCl salt (2249 mg, 11.7 mmol) & NMM (1121 uL, 10.2 mmol) in dichloromethane (25 mL) was added followed by DMAP (62 mg, 0.51 mmol). The reaction was allowed to stir overnight at room temperature. It was then diluted with dichloromethane to 100 mL and washed with H₂O (100 mL), 10% K₂CO₃ (100 mL) and saturated brine solution (100 mL), back extracted all aqueous washes with dichloromethane (30 mL), dried with MgSO₄, filtered and concentrated. Purification by silica gel chromatography with a dichloromethane/methanol gradient yielded di-tert-butyl(10,25-dioxo-3,6,13,16,19,22,29,32-octaoxa-9,26-diazatetratriacontane-1,34-diyl)dicarbamate (2.57 g).

### Preparation of intermediate 2: N1,N16-bis(2-(2-(2-aminoethoxy)ethoxy)ethyl)-4,7,10,13-tetraoxahexa-decane-1,16-diamide TFA salt

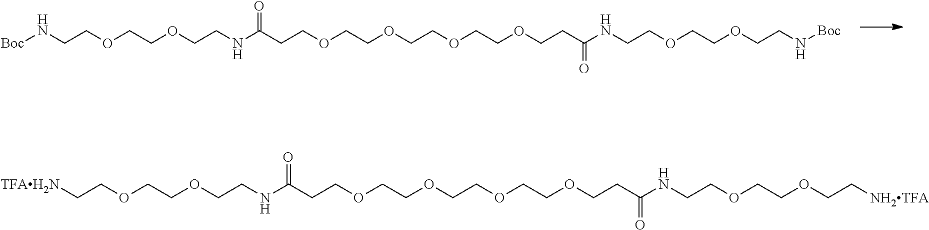

Di-tert-butyl (10,25-dioxo-3,6,13,16,19,22,29,32-octaoxa-9,26-diazatetratriacontane-1,34-diyl)dicarbamate was dissolved in dichloromethane (15 mL) and placed into an ice bath, The round bottom flask was flushed with inert gas and TFA (15 mL) was added. Mixture was allowed to stir for 20 minutes. Afterwards, the reaction mixture was concentrated to yield N1,N16-bis(2-(2-(2-aminoethoxy)ethoxy)ethyl)-4,7,10,13-tetraoxahexadecane-1,16-diamide TFA salt (1885 mg).

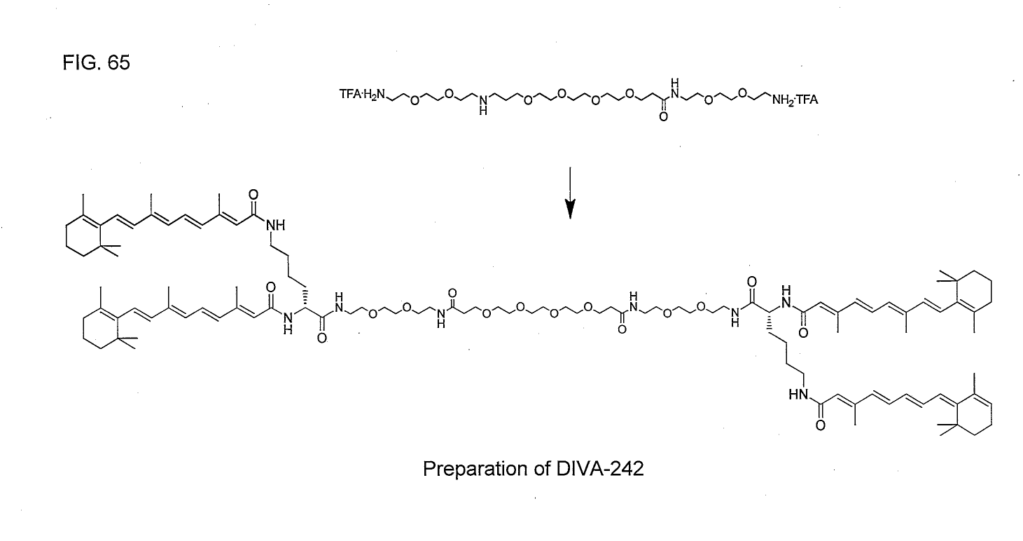

### Preparation of DIVA-242: N1,N16-bis((R,18E,20E,22E,24E)-11-((2E,4E,6E,8E)-3,7-dimethyl-9-(2,6,6-trimethylcyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide (see FIG. 65)

Synthesis of N1,N16-bis((R,18E,20E,22E,24E)-11-((2E,4E,6E,8E)-3,7-dimethyl-9-(2,6,6-trimethylcyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide (DIVA-242) follows the same protocol as diVA from N1,N16-bis(2-(2-(2-aminoethoxy)ethoxy)ethyl)-4,7,10,13-tetraoxahexadecane-1,16-diamide TFA salt. LCMS ESI+: m/z 1940 (M+H).

### Example 27

### In Vitro Efficacy of Fat-Soluble Vitamin Targeting Conjugate

Liposome formulations with 50 nM siRNA were tested. The liposomes were either: HEDC:S104:DOPE:Chol:PEG-DMPE:DiVA (+DiVA) or controls lacking vitamin A moieties (−DiVA) and were incubated in 96-well culture plates containing rat hepatic stellate cells for 30 minutes. After 30 minutes, medium was replaced with fresh growth medium. Forty eight hours later, cells were lysed and gp46 and GAPDH mRNA levels measured by quantitative RT-PCR (TaqMan®) assay, and gp46 levels were normalized to GAPDH levels.

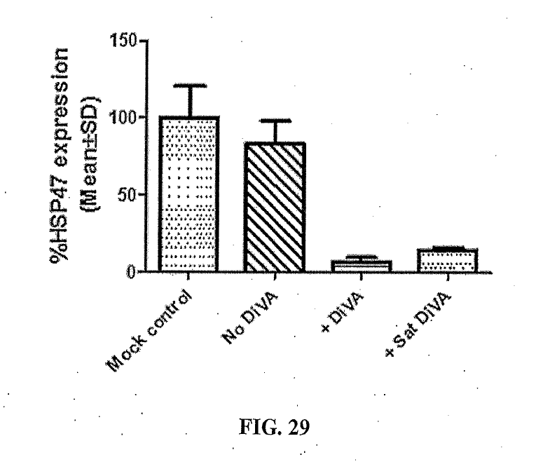

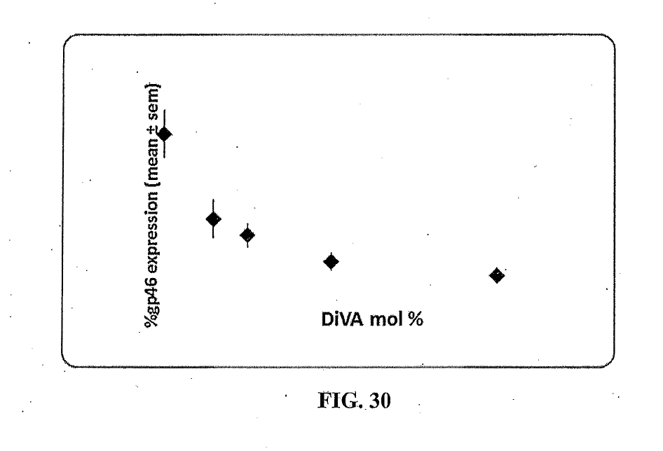

As shown in FIG. 29, in vitro efficacy (pHSC), effect of 2% DiVA siRNA was efficacious with 2% diVA and had an EC₅₀ of 14 nM. This figure shows PHSCs in 96-well plate were incubated with formulation that lacked vitamin A moieties for targeting (−DiVA), or formulation that included vitamin A moieties (+DiVA) at 50 nM siRNA. After 30 minutes, medium was replaced with fresh growth medium. Forty eight hours later, cells were lysed and gp46 and GAPDH mRNA levels measured by quantitative RT-PCR (TaqMan®) assay, and gp46 levels were normalized to GAPDH levels. Normalized gp46 levels were expressed as percent of mock control cells. Error bars indicate standard deviations (n=3). The mean gp46 level following DiVA containing treatment is significantly different from the mock control treatment (P<0.001) based on one-tailed t-test.

Comparison of DiVA and satDiVA

Liposome formulations were transfected into rat pHSCs for 30 min in 96-well plates. After 48 hours, the cells were processed using Cells-to-C® t lysis reagents (Applied Biosystems) and HSP47 mRNA levels were quantified by qRT-PCR. HSP47 expression was normalized mock control. EC₅₀ was determined by measuring HSP47 knockdown (KID) at six half-log doses of siRNA and fitting the data to the “Classic sigmoidal dose response function” in Graphpad Prism® 5.04.

Results show that both DiVA and Sat DiVA increased KD efficacy (Table below, and FIG. 8). The EC₅₀ is 12 nM for DiVA and the EC₅₀ is 14 nM for Sat DiVA.

| Retinoid | | in vitro (pHSC) | in vivo (rat DMNQ) |
|---|---|---|---|
| Conjugate | Formulation | EC₅₀ or % KD | % KD |
| DiVA | 20:20 HEDC:S104 | EC₅₀ = 12 nM | 60% @ 0.75 mpk |
| | with 2% DiVA | | |
| satDiVA | 20:20 HEDC:S104 | EC₅₀ = 14 nM | 74% @ 0.75 mpk |
| | with 2% satDiVA | | |

### Retinoid Conjugate Vs Non-Retinoid Conjugate

Retinoid conjugates were found to be consistently more potent in vitro relative to the non-retinoid equivalents (see 4TTNBB and 4Myr vs. the retinoid conjugate equivalents satDiVA and DiVA).

| Compound | | in vitro (pHSC) |
|---|---|---|
| (Type of Conjugate) | Formulation | EC₅₀ or % KD |
| DiVA (retinoid) | 20:20 HEDC:S104 | 74% @ 50 nM |
| | with 2% DiVA | |
| satDiVA (retinoid) | 20:20 HEDC:S104 | 73% @ 50 nM |
| | with 2% satDiVA | |
| 4TTNPB (non-retinoid) | 20:20 HEDC:S104 | 34% @ 50 nM |
| | with 2% 4TTNPB | |
| 4Myr (non-retinoid) | 20:20 HEDC:S104 | 27% @ 50 nM |
| | with 2% 4Myr | |

### Example 28

### In Vivo Efficacy of Fat-Soluble Vitamin Targeting Conjugate HEDC: S104:DOPE:Chol:PEG-DMPE:diva

In vivo activity of target formulation was evaluated in the short-term liver damage model (referred to as the Quick Model, DMNQ). In this model, short-term liver damage is induced by treatment with a hepatotoxic agent such as dimethylnitrosamine (DMN), and is accompanied by the elevation of gp46 mRNA levels. To induce these changes, male Sprague-Dawley rats were injected intraperitoneally with DMN on six consecutive days. At the end of the DMN treatment period, the animals were randomized to groups based upon individual animal body weight. Formulations were administered as a single IV dose, and given one hour after the last injection of DMN. Twenty four hours later, liver lobes were excised and both gp46 and MRPL19 mRNA levels were determined by quantitative RT-PCR (TaqMan®) assay. mRNA levels for gp46 were normalized to MRPL19 levels.

The results (FIG. 9) show a correlation between the amount of retinoid conjugate and efficacy is evident. Only 0.25 mol % is required to see a significant effect in the rat DMNQ model. With 2 mol % DiVA a robust knockdown of gp46 expression is observed. FIG. 9 shows male Sprague-Dawley rats that were treated with DMN at 10 mg/kg on day 1, 2, 3 and 5 mg/kg on day 4, 5, 6 through intraperitoneal dosing to induce liver damage. Animals (n=8/group) were injected intravenously either with formulations containing 0, 0.25, 0.5, 1, and 2% DiVA at a dose of 0.75 mg/kg siRNA, or PBS (vehicle), one hour after the last injection of DMN. Twenty four hours later, total siRNA was purified from a section of the right liver lobe from each animal and stored at 4° C. until RNA isolation. Control groups included a PBS vehicle group (DMN-treated) and naïve (untreated; no DMN) group. After subtracting background gp46 mRNA levels determined from the naïve group, all test group values were normalized to the average gp46 mRNA of the vehicle group (expressed as a percent of the vehicle group).

Male Sprague Dawley rats (130-160 g) were treated DMN through intraperitoneal dosing to induce liver fibrosis. The DMN treatment regimen was 3 times each week (Mon, Wed, and Fri) with 10 mg/kg (i.e., 5.0 mg/mL of DMN at a dose of 2.0 mL/kg body weight) for first 3 weeks and half dose of 5 mg/kg (i.e., 5 mg/mL of DMN at a dose of 1.0 mL/kg) from day 22 to 57. The sham group animals were injected with PBS (solvent for DMN) using the same schedule. On Day 22, 24 h post the last DMN treatment, blood samples were collected and assayed for liver disease biomarkers to confirm the effectiveness of the DMN treatment. DMN treated animals were assigned to different treatment groups based on body weight and ensure that the mean body weights and the range of body weights of the animals in each group have no significant difference. Animals from pretreatment group were sacrificed on day 25 to evaluate the disease progression stage prior to treatment begins. Treatments with formulations containing gp46 siRNA were started at day 25 with 2 treatments/week at specified siRNA dose for a total of 10 times. On day 59, 48 hours after last formulation treatment and 72 hours after last DMN treatment, animals were sacrificed by CO₂ inhalation. Liver lobes were excised and both gp46 and MRPL19 mRNA levels were determined by quantitative RT-PCR (TaqMan) assay. mRNA levels for gp46 were normalized to MRPL19 levels.

What is claimed is:

1. A pharmaceutical composition comprising a collagen-reducing substance in an amount effective for regenerating normal tissue from fibrotic tissue, and a retinoid in an amount effective for targeting collagen-producing cells, wherein the retinoid is provided as a compound consisting of the structure (retinoid)ₘ-linker-(retinoid)ₙ, wherein m and n are independently 1, 2, or 3, and wherein the linker comprises a polyethylene glycol (PEG) or PEG-like molecule.

2. The pharmaceutical composition according to claim 1, wherein the collagen-reducing substance is selected from the group consisting of a suppressor of collagen production by collagen-producing cells, a promoter of collagen decomposition, and a suppressor of a collagen decomposition inhibitor.

3. (canceled)

4. The pharmaceutical composition according to claim 1, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

5. The pharmaceutical composition according to claim 1, wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

6. The pharmaceutical composition according to claim 1, wherein the compound is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

7. The pharmaceutical composition according to claim 6, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

8. The pharmaceutical composition according to claim 7, wherein the compound is of formula 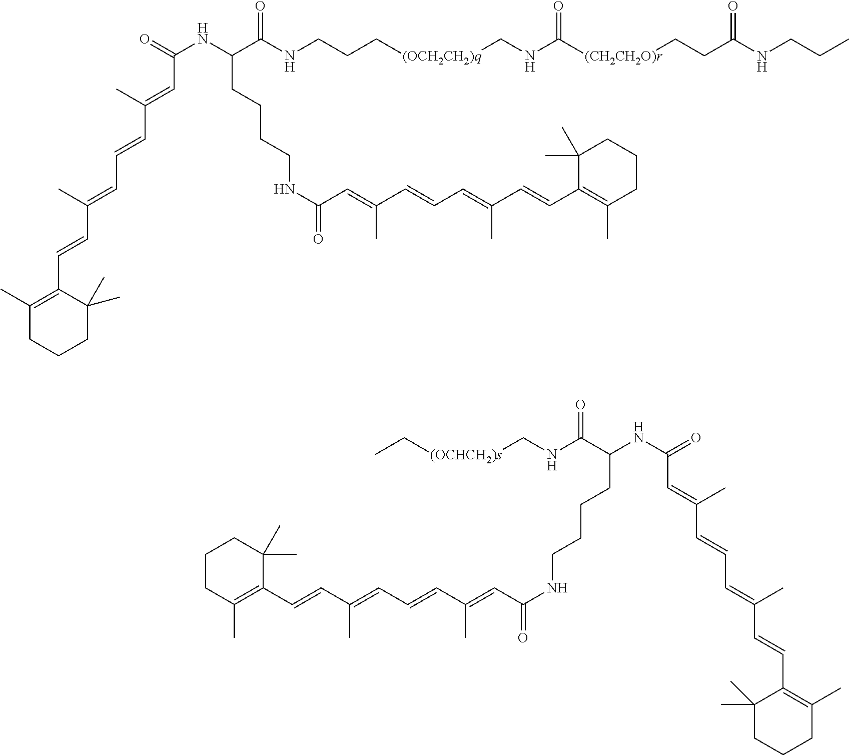
wherein q, r, and s are each independently 1, 2, 3, 4, 5, 6, 7, 8, 9, or 10.

9. The pharmaceutical composition according to claim 7, wherein the compound is of formula

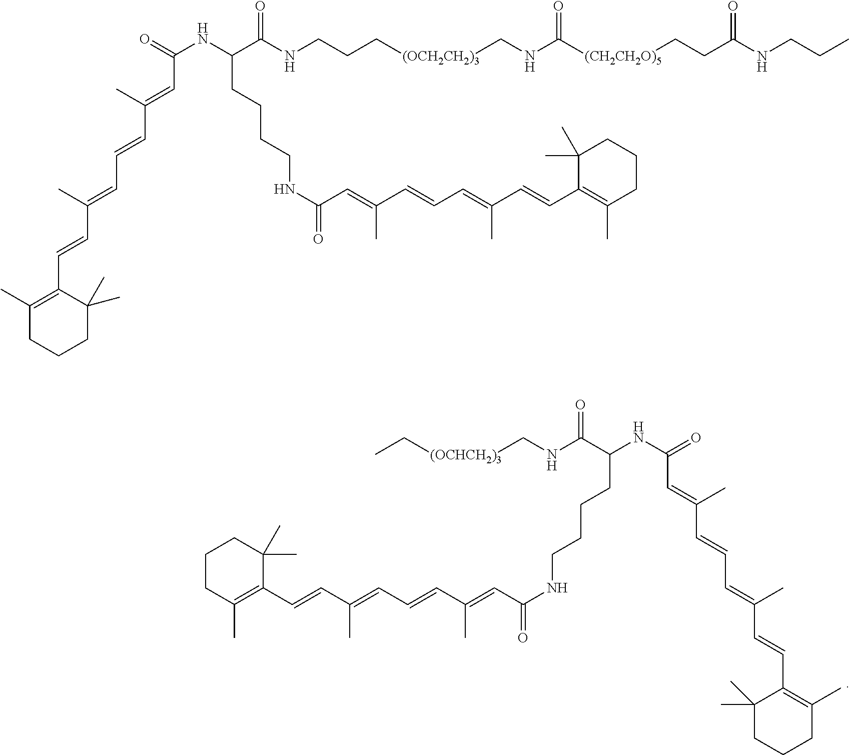

10. The pharmaceutical composition according to claim 1, wherein the PEG is monodisperse.

11. The pharmaceutical composition according to claim 1, wherein the retinoid is provided as a compound consisting of the structure (lipid)ₘ-linker-(retinoid)ₙ, wherein m and n are independently 0, 1, 2, or 3, except that m and n are not both zero; and wherein the linker comprises a polyethylene glycol (PEG) molecule.

12. The pharmaceutical composition according to claim 11, wherein the lipid is selected from one or more of the group consisting of DODC, DSPE, DOPE, and DC-6-14.

13. The pharmaceutical composition according to claim 11, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

14. The pharmaceutical composition according to claim 11, wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

15. The pharmaceutical composition according to claim 14, wherein the compound is selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

16. The pharmaceutical composition according to claim 1, wherein the fibrotic tissue continually receives a fibrotic stimulus.

17. The pharmaceutical composition according to claim 1, wherein regeneration of normal tissue from fibrotic tissue occurs in a space for the growth and differentiation of stem cells, the space being formed by a reduction of collagen accumulated in the fibrotic tissue.

18. The pharmaceutical composition according to claim 2, wherein the suppressor of collagen production by collagen-producing cells is selected from the group consisting of a TGFβ inhibitor, HGF or a substance promoting the production thereof, a PPARγ ligand, an angiotensin inhibitor, a PDGF inhibitor, relaxin or a substance promoting the production thereof, a substance that inhibits the production and secretion of an extracellular matrix component, a cell activity suppressor, a cell growth suppressor, and an apoptosis-inducing substance.

19. The pharmaceutical composition according to claim 2, wherein the promoter of collagen decomposition is collagenase or a collagenase production promoter.

20. The pharmaceutical composition according to claim 2, wherein the suppressor of a collagen decomposition inhibitor is a TIMP inhibitor.

21. The pharmaceutical composition according to claim 18, wherein the substance that inhibits the production and secretion of an extracellular matrix component is a HSP47 inhibitor.

22. The pharmaceutical composition according to claim 1, wherein m+n=3−6.

23. The pharmaceutical composition according to claim 1, wherein m+n=4−6.


SEQUENCE LISTING (8 sequences)

SEQ ID NO: 1 - DNA, 21 bp, Artificial Sequence
  Synthetic oligonucleotide
ggacaggccucuacaacuatt

SEQ ID NO: 2 - DNA, 21 bp, Artificial Sequence
  Synthetic oligonucleotide
uaguuguagaggccugucctt

SEQ ID NO: 3 - DNA, 21 bp, Artificial Sequence
  Synthetic oligonucleotide
ggacaggccuguacaacuatt

SEQ ID NO: 4 - DNA, 21 bp, Artificial Sequence
  Synthetic oligonucleotide
uaguuguacaggccugucctt

SEQ ID NO: 5 - RNA, 27 nt, Artificial sequence
  Synthetic oligonucleotide
guuccaccauaagaugguagacaacag

SEQ ID NO: 6 - RNA, 27 nt, Artificial sequence
  Synthetic oligonucleotide
guugucuaccaucuuaugguggaacau

SEQ ID NO: 7 - RNA, 27 nt, Artificial sequence
  Synthetic oligonucleotide
cgauucgcuagaccggcuucauugcag

SEQ ID NO: 8 - RNA, 27 nt, Artificial sequence
  Synthetic oligonucleotide
gcaaugaagccggucuagcgaaucgau